(19) United States
(12) Patent Application Publication
(10) Pub. No.: US 2010/0166819 A1
(43) Pub. Date: Jul. 1, 2010

(54) Transforming Growth Factor Modulators

(75) Inventors: Wen-Cherng Lee, Lexington, MA (US); Lihong Sun, Lexington, MA (US); Michael Hoemann, Marlborough, MA (US); Deqiang Niu, Lexington, MA (US); Dingxue Yan, Grafton, MA (US); Claudio Chuaqui, Arlington, MA (US); Russell Petter, Stow, MA (US); Helen (Xiaomei) Feng, Winchester, MA (US)

Correspondence Address: Honigman Miller Schwartz and Cohn LLP/BIIB, 350 East Michigan Avenue, Suite 300, Kalamazoo, MI, 49007-3800, US

(73) Assignee: Biogen Idec MA Inc. a corporation

(21) Appl. No.: 12/086,954

(22) PCT Filed: Dec. 22, 2006
(86) PCT No.: PCT/US2006/049169

§ 371 Date: Jul. 29, 2009

Related U.S. Application Data
(60) Provisional application No. 60/752,779, filed on Dec. 22, 2005.

(51) Int. Cl. A61K 31/55; A61K 9/00; A61K 31/502; A61K 31/517; A61K 31/438; C07D 487/10; C07D 401/14; C07D 403/14; A61P 35/00; A61P 9/12; A61P 9/10; A61P 27/02; A61P 1/16; A61P 13/12

(52) U.S. Cl. 424/422; 514/212.02; 514/249; 514/266.21; 514/278; 540/543; 544/230; 546/15; 546/17; 546/18

(57) ABSTRACT

The invention is related to compounds of formula (I) that can be used as antagonists of the TGFβ family type I receptors, Alk5 and/or Alk4, compositions and methods of use. The compounds of formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

This application claims the benefit of the U.S. Provisional application No. 60/752,779, filed on Dec. 22, 2005, which is incorporated herein by reference.

### FIELD OF THE INVENTION

The invention relates to tricyclic spiro compounds, including intermediates for producing them, and methods of using the compounds to modulate the Transforming Growth Factor beta (β) signaling activity.

### BACKGROUND OF THE INVENTION

TGFβ (Transforming Growth Factor β) is a member of a large family of dimeric polypeptide growth factors that includes, for example, activins, inhibins, bone morphogenetic proteins (BMPs), growth and differentiation factors (GDFs) and mullerian inhibiting substance (MIS). TGFβ exists in three isoforms (TGFβ1, TGFβ2, and TGFβ3) and is present in most cells, along with its receptors. Each isoform is expressed in both a tissue-specific and developmentally regulated fashion. Each TGFβ isoform is synthesized as a precursor protein that is cleaved intracellularly into a C-terminal region (latency associated peptide (LAP)) and an N-terminal region known as mature or active TGFβ. LAP is typically non-covalently associated with mature TGFβ prior to secretion from the cell. The LAP-TGFβ complex cannot bind to the TGFβ receptor S and is not biologically active. TGFβ is generally released (and activated) from the complex by a variety of mechanisms including, for example, interaction with thrombospondin-1 or plasmin.

Following activation, TGFβ binds at high affinity to the type II receptor (TGFβRII), a constitutively active serine/threonine kinase. The ligand-bound type II receptor phosphorylates the TGFβ type I receptor (Alk5) in a glycine/serine rich domain, which allows the type I receptor to recruit and phosphorylate downstream signaling molecules, Smad2, or Smad3. See, e.g., Huse, M. et al., Mol. Cell., 8: 671-682 (2001). Phosphorylated Smad2 or Smad3 can then complex with Smad4, and the entire hetero-Smad complex translocates to the nucleus and regulates transcription of various TGFβ-responsive genes. See, e.g., Massagué, J., Ann. Rev. Biochem. Med., 67: 773 (1998).

Activins are also members of the TGFβ superfamily, which are distinct from TGFβ in that they are homo- or heterodimers of activin βa or βb. Activins signal in a manner similar to TGFβ, i.e., by binding to a constitutive serine-threonine receptor kinase, activin type II receptor (ActRIIB), and activating a type I serine-threonine receptor, Alk4, to phosphorylate Smad2 or Smad3. The consequent formation of a hetero-Smad complex with Smad4 also results in the activin-induced regulation of gene transcription.

Indeed, TGFβ and related factors such as activins regulate a large array of cellular processes, e.g., cell cycle arrest in epithelial and hematopoietic cells, control of mesenchymal cell proliferation and differentiation, inflammatory cell recruitment, immunosuppression, wound healing, and extracellular matrix production. See, e.g., Massagué, J., Ann. Rev. Cell. Biol., 6: 594-641 (1990); Roberts, A. B. and Sporn, M. B., Peptide Growth Factors and Their Receptors, 95: 419-472, Berlin: Springer-Verlag (1990); Roberts, A. B. and Sporn, M. B., Growth Factors 8:1-9 (1993); and Alexandrow, M. G. and Moses, H. L., Cancer Res., 55: 1452-1457 (1995). Hyperactivity of TGFβ signaling pathway underlies many human disorders (e.g., excess deposition of extracellular matrix, an abnormally high level of inflammatory responses, fibrotic disorders, and progressive cancers). Similarly, activin signaling and overexpression of activin is linked to pathological disorders that involve extracellular matrix accumulation and fibrosis (see, e.g., Matsuse, T. et al., Am. J. Respir. Cell Mol. Biol., 13: 17-24 (1995); Inoue, S. et al., Biochem. Biophys. Res. Comm., 205: 441-448 (1994); Matsuse, T. et al., Am. J. Pathol., 148: 707-713 (1996); De Bleser et al., Hepatology, 26: 905-912 (1997); Pawlowski, J. E., et al., J. Clin. Invest., 100: 639-648 (1997); Sugiyama, M. et al., Gastroenterology, 114: 550-558 (1998); Munz, B. et al., EMBO J., 18: 5205-5215 (1999)), inflammatory responses (see, e.g., Rosendahl, A. et al., Am. J. Repir. Cell Mol. Biol., 25: 60-68 (2001)), cachexia or wasting (see, e.g., Matzuk, M. M. et al., Proc. Nat. Acad. Sci. USA, 91: 8817-8821 (1994); Coerver, K. A. et al., Mol. Endocrinol., 10: 534-543 (1996); and Cipriano, S. C. et al., Endocrinology, 141: 2319-27 (2000)), diseases of or pathological responses in the central nervous system (see, e.g., Logan, A. et al., Eur. J. Neurosci., 11: 2367-2374 (1999); Logan, A. et al., Exp. Neurol., 159: 504-510 (1999); Masliah, E. et al., Neurochem. Int., 39: 393-400 (2001); De Groot, C. J. A. et al., J. Neuropathol. Exp. Neural., 58: 174-187 (1999); and John, G. R. et al., Nat. Med., 8: 1115-21 (2002)) and hypertension (see, e.g., Dahly, A. J. et al., Am. J. Physiol. Regul. Integr. Comp. Physiol., 283: R757-67 (2002)). Studies have also shown that TGFβ and activin can act synergistically to induce extracellular matrix production (see, e.g., Sugiyama, M. et al., Gastroenterology, 114: 550-558, (1998)). It is therefore desirable to develop modulators (e.g., antagonists) to members of the TGFβ family to prevent and/or treat disorders involving this signaling pathway.

### SUMMARY OF THE INVENTION

The invention is in part based on the discovery that compounds of formula (I) are potent antagonists of the TGFβ family type I receptors, Alk5 and/or Alk4. Thus, compounds of formula (I) can be employed in the prevention and/or treatment of diseases such as fibrosis (e.g., renal fibrosis, pulmonary fibrosis, and hepatic fibrosis), progressive cancers, or other diseases for which reduction of TGFβ family signaling activity is desirable.

Accordingly, one aspect of the present invention features compounds of formula (I):

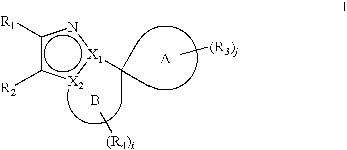

wherein the variables R₁, R₂, R₃, R₄, X₁, X₂, i, and j are described herein.

N-oxide derivatives and pharmaceutically acceptable salts of each of the compounds of formula (I) are also within the scope of this invention. For instance, a ring nitrogen atom of the imidazole core ring or a nitrogen-containing heterocyclic substituent can form an oxide in the presence of a suitable oxidizing agent such as m-chloroperbenzoic acid or H₂O₂.

A compound of formula (I) that is acidic in nature, e.g., by having a carboxyl or phenolic hydroxyl group, can form a pharmaceutically acceptable salt such as a sodium, potassium, calcium, or gold salt. Also within the scope of the invention are salts formed with pharmaceutically acceptable amines such as ammonia, alkyl amines, hydroxyalkylamines, and N-methylglycamine. A compound of formula (I) in its free base form can also be treated with a sufficient amount of acid to form an acid addition salt (e.g., a hydrochloride salt). Examples of such an acid include hydrochloric acid, hydrobromic acid, hydroiodic acid, sulfuric acid, methanesulfonic acid, phosphoric acid, p-bromophenyl-sulfonic acid, carbonic acid, succinic acid, citric acid, benzoic acid, oxalic acid, malonic acid, salicylic acid, malic acid, fumaric acid, ascorbic acid, maleic acid, acetic acid, and other mineral and organic acids well known to those skilled in the art. Conversely, an acid addition salt of formula (I) can be converted back to its free base form, e.g., by treating the salt with a suitable dilute aqueous basic solution (e.g., sodium hydroxide, sodium bicarbonate, potassium carbonate, or ammonia).

Compounds of formula (I) may also take the form of addition salts, for example methiodide or benzylbromide.

Compounds of formula (I) can also be, e.g., in a form of achiral compounds, racemic mixtures, optically active compounds, pure diastereomers, or a mixture of diastereomers.

Compounds of formula (I) exhibit high affinity to the TGFβ family type I receptors, Alk5 and/or Alk4, e.g., with IC₅₀ and Kᵢ values of less than 10 μM under conditions as described below in the Examples. Some compounds of formula (I) exhibit ICₛₒ and Kᵢ values of less than 1 μM (such as below 50 nM). Compounds of formula (I) can also be modified by appending appropriate functionalities to enhance selective biological properties. Such modifications are known in the art and include those that increase biological penetration into a given biological system, tissue, or organ (e.g., blood, lymphatic system, and central nervous system), increase oral availability, increase solubility to allow administration by injection, alter metabolism, and/or alter rate of excretion. Examples of these modifications include, but are not limited to, esterification with polyethylene glycols, derivatization with pivolates or fatty acid substituents, conversion to carbamates, hydroxylation of aromatic rings, and heteroatom-substitution in aromatic rings.

The present invention also features a pharmaceutical composition including a compound of formula (I) (or a combination of two or more compounds of formula (I)) and at least one pharmaceutically acceptable carrier, as well as the use thereof. Further included in the present invention are medicament compositions including any of the compounds of formula (I), alone or in a combination, together with a suitable excipient, and use of one or more compounds of formula (I) for the manufacture of such a medicament composition.

The invention further features a method of inhibiting the TGFβ family type I receptors, Alk5 and/or Alk4 (e.g., with an IC₅₀ value of less than 10 μM; such as, less than 1 μM; and for example, less than 50 nM) in a cell, including the step of contacting the cell with an effective amount of one or more compounds of formula (I). Also within the scope of the invention is a method of inhibiting the TGFβ and/or activin signaling pathway in a cell or in a subject (e.g., a mammal such as a human), including the step of contacting the cell with or administering to the subject an effective amount of one or more compounds of formula (I).

Also within the scope of the present invention is a method of treating a subject or preventing a subject from suffering a condition characterized by or resulting from an elevated level of TGFβ and/or activin activity. The method includes the step of administering to the subject an effective amount of one or more compounds of formula (I). The conditions include, for example, an accumulation of excess extracellular matrix; a fibrotic condition (e.g., atherosclerosis, corneal scarring, keloids, sarcoidosis, spinal cord injury, glomerulonephritis, diabetic nephropathy, hypertensive nephropathy, lupus nephropathy or nephritis, systemic lupus erythematosus, Wegener's granulomatosis, hepatitis-induced cirrhosis, biliary fibrosis, scleroderma, pulmonary fibrosis, idiopathic pulmonary fibrosis, hepatic fibrosis, renal fibrosis, post-infarction cardiac fibrosis, post-surgical fibrosis, radiation-induced fibrosis, fibrosclerosis, fibrotic cancers, fibroids, fibroma, fibroadenomas, or fibrosarcomas); TGFβ-induced metastasis of tumor cells; mesothelioma; and carcinomas (e.g, carcinomas of the lung, breast, liver, biliary tract, gastrointestinal tract, head, neck, pancreas, prostate, cervix, multiple myeloma, melanoma, glioma, or glioblastomas).

Also within the scope of this invention is an implantable device which includes a compound of formula (I) as described above. This device can be in a form known in the art, e.g., a delivery pump or a stent, and can be used for treating or preventing diseases or disorders implicated by TGFβ, e.g., a fibrotic condition.

### DETAILED DESCRIPTION OF THE INVENTION

### I. Definitions

### A. Terms

As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito (1999), and “Advanced Organic Chemistry,” 5th Ed. (Eds.: Smith, M. B. and March, J.), John Wiley & Sons, New York (2001), the entire contents of which are hereby incorporated by reference.

The term “modulating” as used herein means increasing or decreasing, e.g. activity, by a measurable amount. Compounds that modulate TGFβ activity by increasing the activity of the TGFβ receptors are called agonists. Compounds that modulate TGFβ activity by decreasing the activity of the TGFβ receptors are called antagonists. An agonist interacts with a muscarinic receptor to increase the ability of the receptor to transduce an intracellular signal in response to endogenous ligand binding. An antagonist interacts with a TGFβ receptor and competes with the endogenous ligand(s) or substrate(s) for binding site(s) on the receptor to decrease the ability of the receptor to transduce an intracellular signal in response to endogenous ligand binding.

As used herein, the term “aliphatic’ encompasses the terms “alkyl,” “alkenyl,” and “alkynyl,” each of which being optionally substituted as set forth below.

As used herein, an “alkyl” group refers to a saturated aliphatic hydrocarbon group containing 1-8 (e.g., 1-6 or 1-4) carbon atoms. An alkyl group can be straight or branched. Examples of alkyl groups include, but are not limited to, methyl, ethyl, propyl, isopropyl, butyl, isobutyl, sec-butyl, ten-butyl, n-pentyl, n-heptyl, or 2-ethylhexyl. An alkyl group can be substituted (i.e., optionally substituted) with one or more substituents such as halo, cycloaliphatic, heterocycloaliphatic, aryl, heteroaryl, alkoxy, aroyl, heteroaroyl, nitro, cyano, amino, amido, acyl (e.g., cycloaliphaticcarbonyl, (heterocycloaliphatic)carbonyl), sulfonyl, sulfinyl, sulfanyl, sulfoxy, urea, thiourea, sulfamoyl, sulfamide, thioxo, oxo, carboxy, carbamoyl, cycloaliphaticoxy, heterocycloaliphaticoxy, aryloxy, heteroaryloxy, aralkyloxy, heteroarylalkoxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, hydroxyalkyl, aralkyl, (alkoxyaryl)alkyl, (sulfonylamino)alkyl (such as (alkylsulfonylamino)alkyl), aminoalkyl, amidoalkyl, (cycloaliphatic)alkyl, cyanoalkyl, or haloalkyl.

As used herein, an “alkenyl” group refers to an aliphatic carbon group that contains 2-8 (e.g., 2-6 or 2-4) carbon atoms and at least one double bond. Like an alkyl group, an alkenyl group can be straight or branched. Examples of an alkenyl group include, but are not limited to, allyl, isoprenyl, 2-butenyl, and 2-hexenyl. An alkenyl group can be optionally substituted with one or more substituents such as halo, cycloaliphatic, heterocycloaliphatic, aryl, heteroaryl, alkoxy, aroyl, heteroaroyl, nitro, cyano, amino, amido, acyl (e.g., cycloaliphaticcarbonyl, (heterocycloaliphatic)carbonyl), sulfonyl, sulfinyl, sulfanyl, sulfoxy, urea, thiourea, sulfamoyl, sulfamide, thioxo, oxo, carboxy, carbamoyl, cycloaliphaticoxy, heterocycloaliphaticoxy, aryloxy, heteroaryloxy, aralkyloxy, heteroarylalkoxy, or hydroxy.

As used herein, an “alkynyl” group refers to an aliphatic carbon group that contains 2-8 (e.g., 2-6 or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as halo, cycloaliphatic, heterocycloaliphatic, aryl, heteroaryl, alkoxy, aroyl, heteroaroyl, nitro, cyano, amino, amido, acyl (e.g., cycloaliphaticcarbonyl, (heterocycloaliphatic)carbonyl), sulfonyl, sulfinyl, sulfanyl, sulfoxy, urea, thiourea, sulfamoyl, sulfamide, thioxo, oxo, carboxy, carbamoyl, cycloaliphaticoxy, heterocycloaliphaticoxy, aryloxy, heteroaryloxy, aralkyloxy, heteroarylalkoxy, or hydroxy.

As used herein, an “amido” encompasses both “aminocarbonyl” and “carbonylimino.” These terms when used alone or in connection with another group refers to an amido group such as N(R^{X})₂—C(O)— or R^{Y}C(O) —N(R^{X})— when used terminally and —C(O)—N(R^{X})— or —N(R^{X})—C(O)— when used internally, wherein R^{X} and R^{Y} are defined below. Examples of amido groups include alkylamido (such as alkylcarbonylamino and alkylcarbonylamino), (heterocycloaliphatic)amido, (heteroaralkyl)amido, (heteroaryl)amido, (heterocycloalkyl)alkylamido, arylamido, aralkylamido, (cycloalkyl)alkylamido, and cyclo alkylamido

As used herein, an “amino” group refers to —NR^{X}R^{Y} wherein each of R^{X} and R^{Y} is independently hydrogen, alkyl, cycloaliphatic, aryl, heterocycloaliphatic, or heteroaryl, each of which being defined herein and being optionally substituted. Examples of amino groups include alkylamino, dialkylamino, and arylamino.

When the term “amino” is not the terminal group (e.g., alkylcarbonylamino), it is represented by —NR^{X}—. R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl), bicyclic (e.g., indenyl, naphthalenyl, tetrahydronaphthyl, tetrahydroindenyl), and tricyclic (e.g., fluorenyl tetrahydrofluorenyl, or tetrahydroanthracenyl, anthracenyl). The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic (e.g., alkyl, alkenyl, or alkynyl), cycloaliphatic,(cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic ring of a benzofused bicyclic or tricyclic aryl), nitro, carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, and (heteroaraliphatic)carbonyl), sulfonyl (e.g., aliphaticsulfonyl and aminosulfonyl), sulfinyl (e.g., aliphaticsulfinyl), sulfanyl (e.g., aliphaticsulfanyl), cyano, halo, hydroxyl, sulfoxy, urea, thiourea, sulfamoyl, sulfamide, and carbamoyl. Alternatively, an aryl can be unsubstituted.

Non-limiting examples of substituted aryls include haloaryl (e.g., mono-, di (such as p,m-dihaloaryl), and (trihalo)aryl), (carboxy)aryl (e.g., (alkoxycarbonyl)aryl, ((aryalkyl)carbonyloxy)aryl, and (alkoxycarbonyl)aryl), (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl), aminoaryl (e.g., ((alkylsulfonyl)amino)aryl and ((dialkyl)amino)aryl), (cyanoalkyl)aryl, (alkoxy)aryl, (sulfamoyl)aryl (e.g., (aminosulfonyl)aryl), (alkylsulfonyl)aryl, (cyano)aryl, (hydroxyalkyl)aryl, ((alkoxy)alkyl)aryl, (hydroxyl)aryl, ((carboxy)alkyl)aryl, (((dialkyl)amino)alkyl)aryl, (nitroalkyl)aryl, (((alkylsulfonyl)amino)alkyl)aryl, ((heterocycloaliphatic)carbonyl)aryl, ((alkylsulfonyl)alkyl)aryl, (cyanoalkyl)aryl, (hydroxyalkyl)aryl, (alkylcarbonyl)aryl, alkylaryl, (trihaloalkyl)aryl, p-amino-m-alkoxycarbonylaryl, p-amino-m-cyanoaryl, p-halo-m-aminoaryl, and (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic ring of a benzofused bicyclic or tricyclic aryl), nitro, carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, and (heteroaraliphatic)carbonyl), sulfonyl (e.g., aliphaticsulfonyl and aminosulfonyl), sulfinyl (e.g., aliphaticsulfinyl), sulfanyl (e.g., aliphaticsulfanyl), cyano, halo, hydroxyl, sulfoxy, urea, thiourea, sulfamoyl, sulfamide, and carbamoyl.

As used herein, a “bicyclic ring system” includes 8-12 (e.g., 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, “cyclic moiety” includes cycloaliphatic, heterocycloaliphatic, aryl, or heteroaryl, each of which has been defined previously.

As used herein, a “cycloaliphatic” group encompasses a “cycloalkyl” group and a “cycloalkenyl” group, each of which being optionally substituted as set forth below.

As used herein, a “cycloalkyl” group refers to an aliphatic carbocyclic ring of 3-10 (e.g., 4-8) carbon atoms. Examples of cycloalkyl groups include cyclopropyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, and bicyclo[3.2.3]nonyl.

A “cycloalkenyl” group, as used herein, refers to a non-aromatic carbocyclic ring of 3-10 (e.g., 4-8) carbon atoms having one or more double bonds. Examples of a cycloalkenyl group include cyclopentenyl, 1,4-cyclohexa-di-enyl, cycloheptenyl, cyclooctenyl, hexahydro-indenyl, octahydro-naphthyl, bicyclo[2.2.2]octenyl, and bicyclo[3.3.1]nonenyl. A cycloalkenyl group or cycloalkyl group (described above) can be optionally substituted with one or more substituents such as aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic ring of a benzofused bicyclic or tricyclic aryl), nitro, carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, and (heteroaraliphatic)carbonyl), sulfonyl (e.g., aliphaticsulfonyl and aminosulfonyl), sulfinyl (e.g., aliphaticsulfinyl), sulfanyl (e.g., aliphaticsulfanyl), cyano, halo, hydroxyl, sulfoxy, urea, thiourea, sulfamoyl, sulfamide, and carbamoyl.

As used herein, the term “heterocycloaliphatic” encompasses a heterocycloalkyl group, a heterocycloalkenyl group and a heterocycloalkynyl group, each of which being optionally substituted as set forth below.

As used herein, a “heterocycloalkyl” group refers to a 3-10 membered mono- or bicylic (fused or bridged) (e.g., 5- to 10-membered mono- or bicyclic) saturated ring structure, in which one or more of the ring atoms is a heteroatom (e.g., N, O, S, or combinations thereof). Examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.0^{3,7}]nonyl. A monocyclic heterocycloalkyl group can be fused with a phenyl moiety such as tetrahydroisoquinoline. A “heterocycloalkenyl” group, as used herein, refers to a′ mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicycloheteroaliphatics are numbered according to standard chemical nomenclature. A “heterocycloalkynyl” group, as used herein, refers to a 3- to 10-membered (e.g., 4- to 8-membered) non-aromatic ring structure having at least one triple bond between two adjacent ring carbon atoms, and wherein one or more of the ring atoms is a heteroatom, e.g., N, O, or S.

A heterocycloalkyl, heterocycloalkenyl, or heterocycloalkynyl group can be optionally substituted with one or more substituents such as aliphatic (e.g., alkyl, alkenyl, or alkynyl), cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, and (heteroaraliphatic)carbonylamino), nitro, carboxy (e.g., HOOC—, alkoxycarbonyl, and alkylcarbonyloxy), acyl (e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, and (heteroaraliphatic)carbonyl), nitro, cyano, halo, hydroxy, sulfonyl (e.g., alkylsulfonyl and arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring structure having 4 to 15 ring atoms wherein one or more of the ring atoms is a heteroatom (e.g., N, O, S, or combinations thereof) and wherein one or more rings of the bicyclic or tricyclic ring structure is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl,cinnolyl, phthalazyl, quinazolyl, quinoxalyl, isoquinolyl, 4H-quinolizyl, benzo-1,2,5-thiadiazolyl, or 1,8-naphthyridyl. Without limitation, examples of a monocyclic heteroaryl include furyl, thiophenyl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4H-pranyl, pyridyl, pyridazyl, pyrimidyl, pyrazolyl, pyrazyl, or 1,3,5-triazyl. Monocyclic heteroaryls are numbered according to standard chemical nomenclature. Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic or heterocyclic ring of a bicyclic or tricyclic heteroaryl), nitro, carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic) aliphatic)carbonyl, and (heteroaraliphatic)carbonyl), sulfonyl (e.g., aliphaticsulfonyl and aminosulfonyl), sulfinyl (e.g., aliphaticsulfinyl), sulfanyl (e.g., aliphaticsulfanyl), cyano, halo, hydroxyl, sulfoxy, urea, thiourea, sulfamoyl, sulfamide, or carbamoyl. Alternatively, a heteroaryl can be unsubstituted.

Non-limiting examples of substituted heteroaryls include (halo)heteroaryl (e.g., mono- and di-(halo)heteroaryl), (carboxy)heteroaryl (e.g., (alkoxycarbonyl)heteroaryl), cyanoheteroaryl, aminoheteroaryl (e.g., ((alkylsulfonyl)amino)heteroaryl and ((dialkyl)amino)heteroaryl), (amido)heteroaryl (e.g., aminocarbonylheteroaryl, ((alkylcarbonyl)amino)heteroaryl, ((((alkyDamino)alkyl)aminocarbonypheteroaryl, (((heteroaryl)amino)carbonyl)heteroaryl, ((heterocycloaliphatic)carbonyl)heteroaryl, and ((alkylcarbonyl)amino)heteroaryl), (cyanoalkyl)heteroaryl, (alkoxy)heteroaryl, (sulfamoyl)heteroaryl (e.g., (aminosulfonyl)heteroaryl), (sulfonyl)heteroaryl (e.g., (alkylsulfonyl)heteroaryl), (hydroxyalkyl)heteroaryl, (alkoxyalkyl)heteroaryl, (hydroxyl)heteroaryl, ((carboxy)alkyl)heteroaryl, [((dialkyl)amino)alkyl]heteroaryl, (heterocycloaliphatic)heteroaryl, (cycloaliphatic)heteroaryl, (nitroalkyl)heteroaryl, (((alkylsulfonyl)amino)alkyl)heteroaryl, ((alkylsulfonyl)alkyl)heteroaryl, (cyanoalkyl)heteroaryl, (acyl)heteroaryl (e.g., (alkylcarbonyl)heteroaryl), (alkyl)heteroaryl, and (haloalkyl)heteroaryl (e.g., trihaloalkylheteroaryl).

A “heteroaraliphatic (such as a heteroaralkyl group) as used herein, refers to an aliphatic group (e.g., a C₁₋₄ alkyl group) that is substituted with a heteroaryl group. “Aliphatic,” “alkyl,” and “heteroaryl” have been defined above.

A “heteroaralkyl” group, as used herein, refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, oxo (on a non-aromatic carbocyclic ring of a benzofused bicyclic or tricyclic aryl), nitro, carboxy, amido, acyl (e.g., aliphaticcarbonyl, (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, and (heteroaraliphatic)carbonyl), sulfonyl (e.g., aliphaticsulfonyl and aminosulfonyl), sulfinyl (e.g., aliphaticsulfinyl), sulfanyl (e.g., aliphaticsulfanyl), cyano, halo, hydroxyl, sulfoxy, urea, thiourea, sulfamoyl, sulfamide, and carbamoyl.

As used herein, a “cyclic moiety” includes cycloalkyl, heterocycloalkyl, cycloalkenyl, heterocycloalkenyl, aryl, or heteroaryl, each of which has been defined previously.

As used herein, an “acyl” group refers to a formyl group or R^{W}—C(O)— (such as alkyl-C(O)—, also referred to as “alkylcarbonyl”) where alkyl has been defined previously and R^{W} is aliphatic, cylcoaliphatic, heterocycloaliphatic, each of which can be optionally substituted with aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, cyano, halo, hydroxyl, sulfoxy, urea, thiourea, sulfamoyl, sulfamide, and carbamoyl. Acetyl and pivaloyl are examples of acyl groups.

As used herein, an “aroyl” (or “arylcarbonyl”) group refers to Ar—CO— wherein Ar is an aryl group and has the same meaning as previously provided.

As used herein a “heteroaroyl” (or “heteroarylcarbonyl”) group refers to HetAr—CO— wherein HetAr is a heteroaryl group and has the same meaning as previously provided.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z} wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X} when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally, or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —S(O)₂—NR^{x}R^{y} or —NR^{x}—S(O)₂—R^{z} when used terminally; or —S(O)₂—NR^{x}— or —NR^{x}—S(O)₂— when used internally, wherein R^{x}, R^{y}, and R^{Z} are defined above.

As used herein a “sulfanyl” group refers to —S—R^{X} when used terminally and encompassed mercapto, and —S— when used internally, wherein R^{X} has been defined above. Examples of sulfanyls include alkyl sulfanyl.

As used herein a “sulfinyl” group refers to —S(O)—R^{X} when used terminally and —S(O)—when used internally, wherein R^{X} has been defined above.

As used herein, a “sulfonyl” group refers to —S(O)₂—R^{X} when used terminally and —S(O)₂— when used internally, wherein R^{X} has been defined above.

As used herein, a “sulfoxy” group refers to —O—SO—R^{X} or —SO—O—R^{X}, when used terminally and —O—S(O)— or —S(O)—O— when used internally, where R^{X} has been defined above.

As used herein, an “alkoxy” group refers to an alkyl-O— group, wherein “alkyl” has the same meaning as defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” group refers to an alkyl group such as alkyl-O-alkyl-, wherein alkyl has been defined above.

As used herein, a “carbonyl” group refer to —C(O)—.

As used herein, an “oxo” group refers to ═O.

As used herein, a “thioxo” group refers to ═S.

As used herein, an “aminoalkyl” group refers to the structure (R^{X})₂N-alkyl-.

As used herein, a “cyanoalkyl” group refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally, and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(NR_{X}R_{Y})N(R_{X}R_{Y}), wherein R_{X} and R_{Y} have the same meanings as defined above.

As used herein, the term “amidino” group refers to the structure —C═(NR_{X})N(R_{X}R_{Y}), wherein R_{X} and R_{Y} have the same meanings defined above.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.2.3]nonyl, 2-oxa-bicyclo[2.2.2]octyl, 1-aza-bicyclo[2.2.2]octyl, 3-aza-bicyclo[3.2.1]octyl, and 2,6-dioxa-tricyclo[3.3.1.03,7]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl is an example of a carboxy group used terminally. A group is internal when the group is present in the middle of a substituent to at the end of the substituent bound to the rest of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an effective amount is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970).

As used herein, a “patient” refers to a mammal, including a human.

An antagonist, as used herein, is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

The phrase “optionally substituted” is used interchangeably with the phrase “substituted or unsubstituted.” As described herein, compounds of the invention may optionally be substituted with one or more substituents, such as are illustrated generally above, or as exemplified by particular classes, subclasses, and species of the invention. As described herein, the variables R₁, R₂, R₃, R₄, X₁, X₂, i, j, and others described in formula (I) encompass specific groups, such as alkyl and aryl. Unless otherwise noted, each of the specific groups for the variables R₁, R₂, R₃, R₄, X₁, X₂, i, j and others described in formula (I) may be optionally substituted with one or more substituents described herein. Each substituent of a specific group is further optionally substituted with one to three halo, cyano, alkoxy, hydroxyl, nitro, haloalkyl, or alkyl groups, or their combinations. For instance, an alkyl group may be substituted with alkylsulfanyl and the alkylsulfanyl may be optionally substituted with one to three halo, oxo, cyano, alkoxy, hydroxyl, nitro, haloalkyl, or alkyl groups, or their combinations. As an additional example, an alkyl may be substituted with a (cycloalkyl)carbonylamino and the cycloalkyl portion of a (cycloalkyl)carbonylamino may be further optionally substituted with one to three halo, cyano, oxo, alkoxy, hydroxyl, nitro, haloalkyl, or alkyl groups, or their combinations.

In general, the term “substituted,” whether preceded by the term “optionally” or not, refers to the replacement of one or more hydrogen radicals in a given structure with the radical of a specified substituent. Specific substituents are described above in the definitions and below in the description of compounds and examples thereof. Unless otherwise indicated, an optionally substituted group may have a substituent at each substitutable position of the group, and when more than one-position in any, given structure may be substituted with more than one substituent selected from a specified group, the substituent may be either the same or different at every position. A ring substituent, such as a heterocycloalkyl, may be bound to another ring, such as a cycloalkyl, to form a spiro-bicyclic ring system, e.g., both rings share one common atom. As one of ordinary skill in the art will recognize, combinations of substituents envisioned by this invention are those combinations that result in the formation of stable or chemically feasible compounds.

The phrase “stable or chemically feasible compounds,” as used herein, refers to compounds that are not substantially altered when subjected to conditions to allow for their production, detection, and preferably their recovery, purification, and use for one or more of the purposes disclosed herein.

As used herein the term “electron withdrawing group” refers to a substituent that draws electrons to itself more than a hydrogen atom would if it occupied the same position in a molecule. See, e.g., March, J., “Advanced Organic Chemistry,” 3rd edition, pp 16-17, Wiley-Interscience, New York. Examples of electron withdrawing groups include but are not limited to —C(O)—, —S(O)—, or —S(O)₂—.

An antagonist, as used herein, is a molecule that binds to the receptor without activating the receptor. It competes with the endogenous ligand(s) or substrate(s) for binding site(s) on the receptor and, thus inhibits the ability of the receptor to transduce an intracellular signal in response to endogenous ligand binding.

Unless otherwise stated, the structures depicted herein are also meant to include all isomeric (e.g., enantiomeric, diastereomeric, and geometric (or conformational)) forms of the structures; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are also within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays:

### B. Abbreviations

The following abbreviations have the following meanings. If an abbreviation is not defined, it has its generally accepted meaning.
BEMP=2-tert-butylimino-2-diethylamino-1,3-dimethylperhydro-1,3,2-diazaphosphorine Boc=t-butoxycarbonyl
BOP=benzotriazol-1-yloxy-tris(dimethylamino)phosphonium hexafluorophosphate
bd=broad doublet
bs=broad singlet
d=doublet
dd=doublet of doublets
DIC=diisopropylcarbodiimide
DMF=dimethylformamide
DMAP=dimethylaminopyridine
DMSO=dimethylsulfoxide
EDC=ethyl-1-(3-dimethyaminopropyl)carbodiimide
eq.=equivalents
EtOAc=ethyl acetate
g=grams
HOBT=1-hydroxybenzotriazole
DIPEA=Hunig's base=diisopropylethylamine
L=liter
LC-MS=liquid chromatography/mass spectroscopy
m=multiplet
M=molar
max=maximum
meq=milliequivalent
mg=milligram
mL=milliliter
mm=millimeter
mmol=millimole
MOC=methoxyoxycarbonyl
N=normal
N/A=not available
ng=nanogram
nm=nanometers
OD=optical density
PEPC=1-(3-(1-pyrrolidinyl)propyl)-3-ethylcarbodiimide
PP-HOBT=piperidine-piperidine-1-hydroxybenzotrizole
psi=pounds per square inch
Ph=phenyl
q=quartet
quint.=quintet
rpm=rotations per minute
s=singlet
t=triplet
TFA=trifluoroacetic acid
THF=tetrahydrofuran
tlc=thin layer chromatography
μL=microliter
UV=ultra-violet

### II. Description of the Compounds

In general, the present invention features compounds of formula (I)

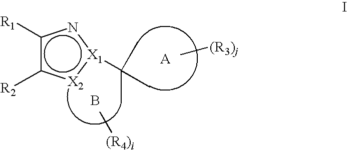

In formula (I),

R₁ is aryl or heteroaryl, each optionally substituted with 1 to 3 Rₐ;

R₂ is aryl or heteroaryl, each optionally substituted with 1 to 3 R_{b};

Ring A is a 5 to 8 membered cycloaliphatic, or a 5 to 8 membered heterocycloaliphatic containing one to three heteroatoms;

Ring B is a 5 to 8 membered heterocycloaliphatic containing one to three heteroatoms;

each of Rₐ and R_{b} is independently an optionally substituted aliphatic, alkoxy; acyl, halo, hydroxy, amino, amido (e.g., aminocarbonyl and alkylcarbonylamino), nitro, cyano, guanadino, amidino, carboxy, sulfo, sulfinyl, sulfonyl, sunfanyl (e.g., mercaptoalkylsulfanyl, cycloalkylsulfanyl, heterocycloalkylsulfanyl, arylsulfanyl, and heteroarylsulfanyl), alkoxycarbonyl, alkylcarbonyloxy, urea, thiourea, sulfamoyl, sulfamide, carbamoyl, cycloalkyl, cycloalkyloxy, heterocycloalkyl, heterocycloalkyloxy, aryl, aryloxy, aroyl, heteroaryl, heteroaryloxy, heteroaroyl, oxo, thioxo, ═N—OR_{f}, —N₃, or ═N—N(R_{f})₂, or

any two of Rₐ or any two of R_{b} on adjacent atoms, together with the atoms to which they are attached, may form a 5- to 8-membered cycloaliphatic or a 5 to 8 membered heterocycloaliphatic;

each R₃ and R₄, if present, is independently aliphatic, acyl, alkoxy, cycloalkyloxy, heterocycloalkyloxy, aryloxy, heteroaryloxy, aralkyloxy, heteroarylalkoxy, amino, amido (e.g., aminocarbonyl, alkylcarbonylamino, cycloalkylcarbonylamino, cycloalkylalkylcarbonylamino, arylcarbonylamino, aralkylcarbonylamino, (heterocycloalkyl)carbonylamino, (heterocycloalkylalkyl)carbonylamino, heteroarylcarbonylamino, and heteroaralkylcarbonylamino), nitro, carboxy (e.g., alkoxycarbonyl and alkylcarbonyloxy), cyano, halo, hydroxyl, sulfanyl (e.g., mercapto and alkylsulfanyl), sulfinyl, sulfonyl, urea, thiourea, sulfamoyl, sulfamide, oxo, thioxo, —N₃, ═N—OR_{f}, or ═N—N(R_{f})₂, or

any two of R₃ or any two of R₄ on the same atom, together with the atom to which they are attached, may form a 3- to 8-membered cycloaliphatic or heterocycloaliphatic ring, or

any two of R₃ or any two of R₄ on adjacent atoms, together with the atoms to which they are attached, may form a 3- to 8-membered cycloaliphatic or a 3- to 8-membered heterocycloaliphatic ring;

each of the optional heteroatoms of Rings A and B are O, S, or N, and the S and N atoms in Rings A and B may form part of the groups —S(O)ₘ— and —N(R_{f})—;

each R_{F} is independently H, alkyl, heteroaryl, aryl, acyl (e.g., alkylcarbonyl), aroyl (e.g., arylcarbonyl), heteroaroyl, amido (e.g., aminocarbonyl), sulfamoyl, sulfamide, or carboxy (e.g., alkoxycarbonyl);

X₁ is C and X₂ is N, or X₁ is N and X₂ is C;

each i is independently 0 to 3;

each j is independently 0 to 3; and

each m is independently 0 to 2.

In one embodiment, X₁ is C and X₂ is N to provide imidazole compounds of the invention.

In another embodiment, X₁ is N and X₂ is C to provide pyrazole compounds of the invention.

In some embodiments R₁ is an optionally substituted aryl, such as an optionally substituted mono- or bi-carbocyclic aromatic group. Each R₁ is an optionally substituted mono-carbocyclic aromatic (“monocyclic aryl”) group, e.g., an optionally substituted phenyl. Each R₁ is an unsubstituted mono-carbocyclic aromatic group, e.g., an unsubstituted phenyl. Each R₁ is an optionally substituted bi-carbocyclic aromatic group, e.g., an optionally substituted naphthyl, indenyl, or azulenyl. Each R₁ is a substituted bi-carbocyclic aromatic group, e.g., a substituted naphthyl, indenyl, or azulenyl. Each R₁ is an unsubstituted bi-carbocyclic aromatic (“bicyclic aryl”) group, e.g., an unsubstituted naphthyl, indenyl, or azulenyl.

In some embodiments R₁ is an optionally substituted heteroaryl, such as a mono- or bi-heterocyclic aromatic group. Each R₁ is an optionally substituted mono-heterocyclic aromatic (“monocyclic heteroaryl”) group, e.g., furanyl, pyrrolyl, oxazolyl, thiazolyl, imidazolyl, pyrazaloyl, isoxazolyl, isothiazolyl, triazolyl, pyridinyl, pyridazinyl, pyrimidinyl, and pyrazinyl, each of which are optionally substituted. Each R₁ is an optionally substituted 5-membered mono-heterocyclic aromatic group, e.g., furanyl, pyrrolyl, oxazolyl, thiazolyl, imidazolyl, pyrazaloyl, isoxazolyl, isothiazolyl, and triazolyl, each of which is optionally substituted. Each R₁ is an optionally substituted 6-membered mono-heterocyclic aromatic group, e.g., pyridinyl, pyridazinyl, pyrimidinyl, and pyrazinyl, each of which is optionally substituted.

In some embodiments R₁ is an optionally substituted bicyclic heteroaryl, e.g., indolizinyl, indolyl, isoindolyl, benzofuranyl, benzothiopenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, naphthyridinyl, and pteridinyl, each of which is optionally substituted. Each R₁ is an optionally substituted 9-membered bi-heterocyclic aromatic group, e.g., indolizinyl, indolyl, isoindolyl, benzofuranyl, benzothiopenyl, 1H-indazolyl, benzimidazolyl, benzthiazolyl, and purinyl, each of which is optionally substituted. Each R₁ is an optionally substituted 10-membered bi-heterocyclic aromatic group, e.g., 4H-quinolizinyl, quinolinyl, isoquinolinyl, cinnolinyl, phthalazinyl, quinazolinyl, quinoxalinyl, naphthyridinyl, and pteridinyl, each of which are optionally substituted. Each R₁ is an optionally substituted benzofused bicyclic aryl moiety covered under the term aryl, e.g., tetrahydronaphthalyl. Each R₁ is an optionally substituted benzofused bicyclic heteroaryl moiety covered under the term heteroaryl, e.g., indolinyl and tetrahydroquinolinyl.

In some embodiments R₁ is an optionally substituted pyridinyl or pyrimidinyl. R₁ is an optionally substituted pyridine-2-yl. R₁ is a pyridine-2-yl substituted with one to three of halo, cyano, alkoxy, hydroxyl, nitro, haloalkyl, and alkyl. R₁ is 6-aliphatic pyridine-2-yl. R₁ is 6-methylpyridine-2-yl.

In some embodiments R₁ is an optionally substituted benzofused bicyclic heteroaryl. R₁ is an optionally substituted 1,3-benzodioxolane.

In some embodiments R₂ is an optionally substituted aryl, such as an optionally substituted mono- or bi-carbocyclic aromatic group. Each R₂ is an optionally substituted mono-carbocyclic aromatic (“monocyclic aryl”) group, e.g., an optionally substituted phenyl. Each R₂ is an unsubstituted mono-carbocyclic aromatic group, e.g., an unsubstituted phenyl. Each R₂ is an optionally substituted bi-carbocyclic aromatic group, e.g., an optionally substituted naphthyl, indenyl, or azulenyl. Each R₂ is a substituted bi-carbocyclic aromatic group, e.g., a substituted naphthyl, indenyl, or azulenyl. Each R₂ is an unsubstituted bi-carbocyclic aromatic (“bicyclic aryl”) group, e.g., an unsubstituted naphthyl, indenyl, or azulenyl.

In some embodiments R₂ is an optionally substituted heteroaryl, such as a mono- or bi-heterocyclic aromatic group. Each R₂ is an optionally substituted mono-heterocyclic aromatic (“monocyclic heteroaryl”) group, e.g., furanyl, pyrrolyl, oxazolyl, thiazolyl, imidazolyl, pyrazaloyl, isoxazolyl, isothiazolyl, triazolyl, pyridinyl, pyridazinyl, pyrimidinyl, and pyrazinyl, each of which are optionally substituted. Each R₂ is an optionally substituted 5-membered mono-heterocyclic aromatic group, e.g., furanyl, pyrrolyl, oxazolyl, thiazolyl, imidazolyl, pyrazaloyl, isoxazolyl, isothiazolyl, and triazolyl, each of which is optionally substituted. Each R₂ is an optionally substituted 6-membered mono-heterocyclic aromatic group, e.g., pyridinyl, pyridazinyl, pyrimidinyl, and pyrazinyl, each of which is optionally substituted.

In some embodiments R₂ is an optionally substituted bicyclic heteroaryl, e.g., indolizinyl, indolyl, isoindolyl, benzofuranyl, benzothiopenyl, 1H-indazolyl, benzimidazolyl, benzthiazolyl, purinyl, 4H-quinolizinyl, quinolinyl, isoquinolinyl, cinnolinyl, phthalazinyl, quinazolinyl, quinoxalinyl, naphthyridinyl, and pteridinyl, each of which is optionally substituted. Each R₂ is an optionally substituted 9-membered bi-heterocyclic aromatic group, e.g., indolizinyl, indolyl, isoindolyl, benzofuranyl, benzothiopenyl, 1H-indazolyl, benzimidazolyl, benzthiazolyl, and purinyl, each of which is optionally substituted. Each R₂ is an optionally substituted 10-membered bi-heterocyclic aromatic group, e.g., 4H-quinolizinyl, quinolinyl, isoquinolinyl, cinnolinyl, phthalazinyl, quinazolinyl, quinoxalinyl, naphthyridinyl, and pteridinyl, each of which are optionally substituted. Each R₂ is an optionally substituted benzofused bicyclic aryl moiety covered under the term aryl, e.g., tetrahydronaphthalyl. Each R₂ is an optionally substituted benzofused bicyclic herteroaryl moiety covered under the term heteroaryl, e.g., indolinyl and tetrahydroquinolinyl.

In some embodiments R₂ is an optionally substituted pyridinyl or pyrimidinyl. R₂ is an optionally substituted pyridine-2-yl. R₂ is a pyridine-2-yl substituted with one to three of halo, cyano, alkoxy, hydroxyl, nitro, haloalkyl, and alkyl. R₂ is 6-aliphatic pyridine-2-yl. R₂ is 6-methylpyridine-2-yl.

In some embodiments R₂ is an optionally substituted bicyclic herteroaryl, such as a benzofused bicyclic heteroaryl. R₂ is an optionally substituted 1,3-benzodioxolane.

Examples of bicyclic and tricyclic heteroaryl R₁ or R₂ substituents include, but are not limited to

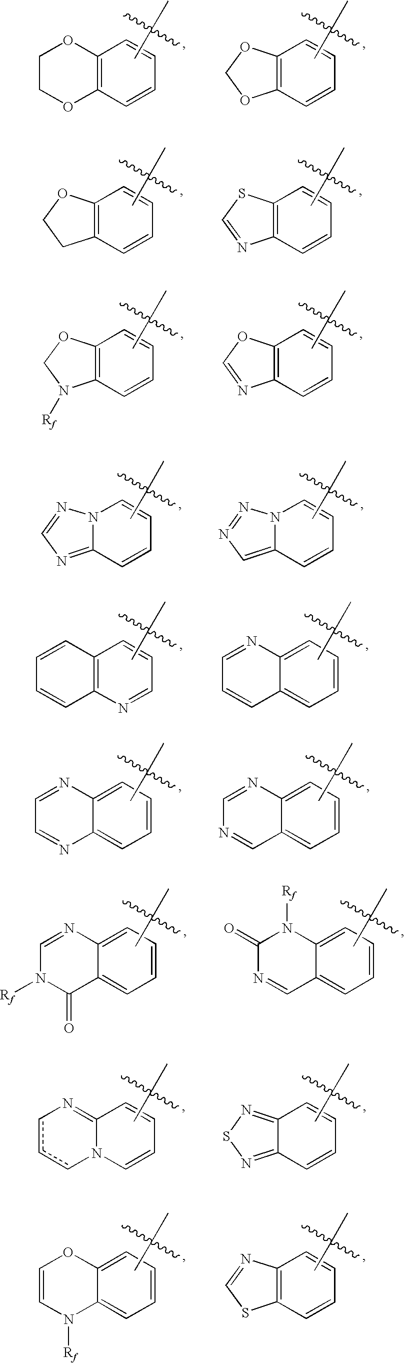

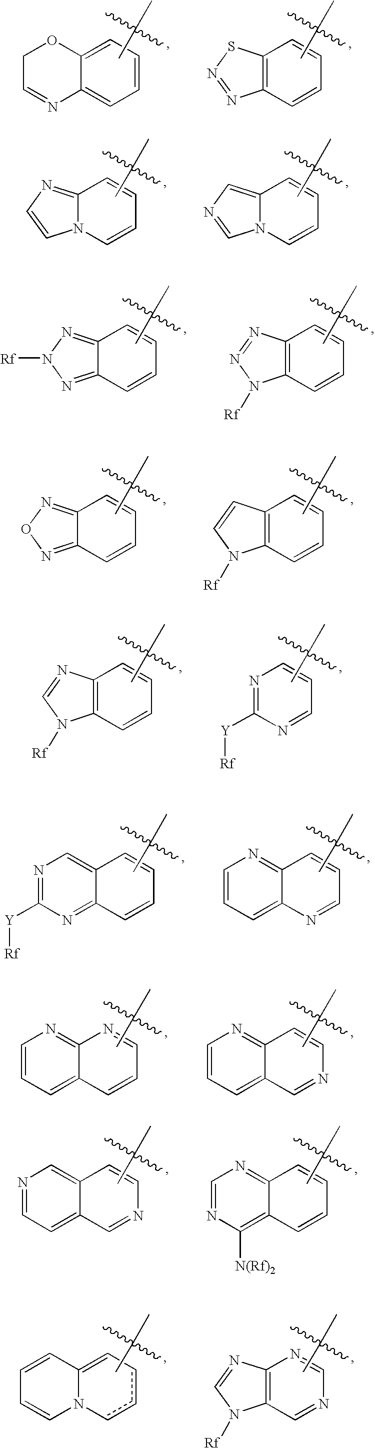

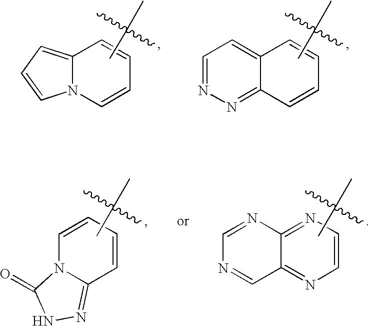

In some embodiments, Ring A is a five or six membered saturated or partially unsaturated cycloaliphatic or heterocycloaliphatic ring wherein Ring B, R₁, R₂, R₃ and R₄ are as previously described. Ring A is a five membered saturated or partially unsaturated cycloaliphatic or heterocycloaliphatic ring. Ring A is a five membered saturated or partially unsaturated cycloaliphatic. Ring A is a five membered saturated or partially unsaturated heterocycloaliphatic ring, such as 2H-pyrrole, 2-pyrroline, 3-pyrroline, pyrrolidine, imidazolidine, 1,3-dioxolane, 2-imidazoline, 2-pyrazoline, and the like. Ring A is six membered saturated or partially unsaturated cycloaliphatic or heterocycloaliphatic ring. Ring A is six membered saturated or partially unsaturated cycloaliphatic. Ring A is six membered saturated or partially unsaturated heterocycloaliphatic ring, such as 2H-pyran, dihyrdopyridine, tetrahyrdopyridine, dihydropyrimidine, tetrahydropyrimidine, piperidine, piperazine, 1,4-dioxane, morpholine, and the like.

In other embodiments, Ring A contains one degree of unsaturation.

In other embodiments, Ring A contains two degrees of unsaturation.

In other embodiments, Ring A is a seven membered cycloaliphatic or heterocycloaliphatic ring.

In some embodiments, Ring A is substituted with at least one R₃. Ring B is substituted with at least one R₃, such as alkoxy, oxo, amino, nitro, cyano, halo, haloalkyl, and hydroxyl. In some embodiments, Ring A substituted with —OR_{f}—N(R_{f})₂, ═O, —N₃ or ═N—OR_{f}. In other embodiments, Ring A is substituted with at least ═O. In certain embodiments, Ring A includes a nitrogen ring atom which is substituted with R_{f}. In some embodiments, Ring A is a lactam.

In some embodiments, Ring B is a five, six or seven membered cycloaliphatic or heterocycloaliphatic ring wherein Ring A, R₁, R₂, R₃ and R₄ are as previously described. In some embodiments, Ring B is a five or six membered cycloaliphatic or heterocycloaliphatic ring. Ring B is a five membered cycloaliphatic. Ring B is a five membered heterocycloaliphatic ring, such as 2H-pyrrole, 2-pyrroline, 3-pyrroline, 2-imidazoline, 2-pyrazoline, pyrrolidine, and the like. Ring B is six membered cycloaliphatic or heterocycloaliphatic ring. Ring B is six membered cycloaliphatic. Ring B is six membered heterocycloaliphatic ring, such as piperidine, piperazine, morpholine, 2H-pyran, 4H-pyran, dihyrdopyridine, tetrahyrdopyridine, dihydropyrimidine, tetrahydropyrimidine, and the like.

In other embodiments, Ring B contains one degree of unsaturation.

In other embodiments, Ring B contains two degrees of unsaturation provided that Ring B is non-aromatic.

In other embodiments, Ring B is a seven membered cycloaliphatic or heterocycloaliphatic ring.

In some embodiments, Ring B is substituted with at least one R₄. Ring B is substituted with at least one R₄, such as alkoxy, amino, nitro, cyano, halo, haloalkyl, and hydroxyl.

All Combinations and permutations of the above embodiments are within the scope of the invention. Thus, for example, in a specific compound R₁ and R₂ can both be heteroaryl, Ring A can be a 6 membered cycloaliphatic and Ring B can be a six membered partially unsaturated heterocycloaliphatic. In some embodiments, at least one each of R₁ and R₂ is heteroaryl.

Non-limiting examples of the invention are provided in Table 1. In these examples, X₁ is C and X₂ is N.

| Ex | R₁ | R₂ | 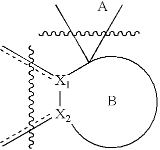 | 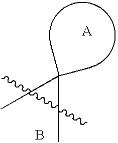 |
|---|---|---|---|---|
| 1 | 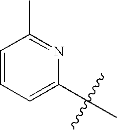 | 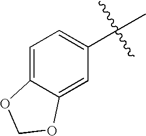 | 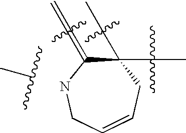 | 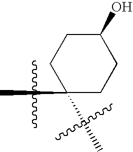 |
| 2 | 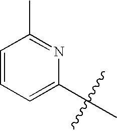 | 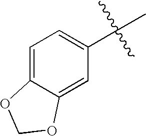 | 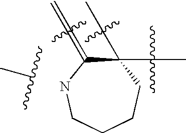 | 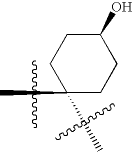 |
| 3 | 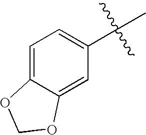 | 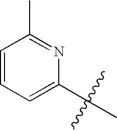 | 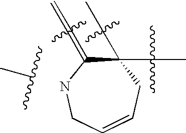 | 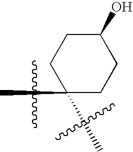 |
| 4 | 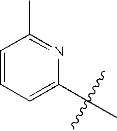 | 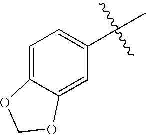 | 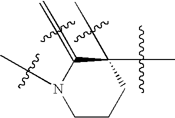 | 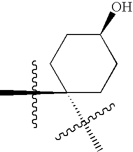 |
| 5 | 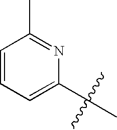 | 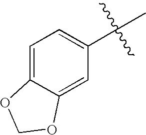 | 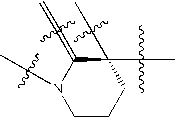 | 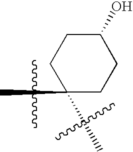 |
| 6 | 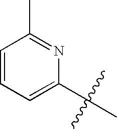 | 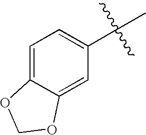 | 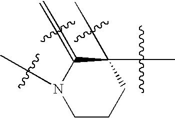 | 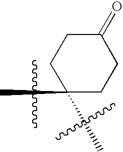 |
| 7 | 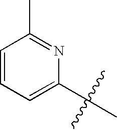 | 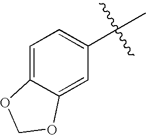 | 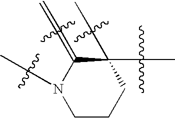 | 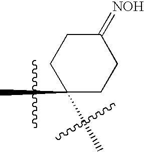 |
| 8 | 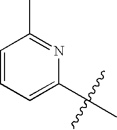 | 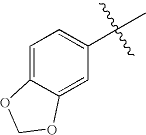 | 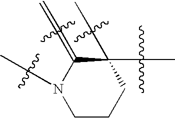 | 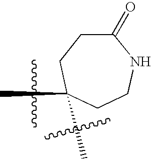 |
| 9 | 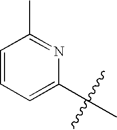 | 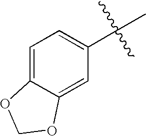 | 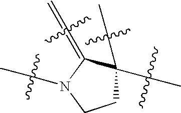 | 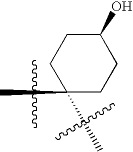 |
| 10 | 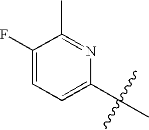 | 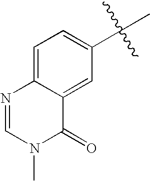 | 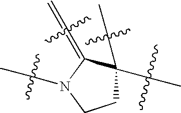 | 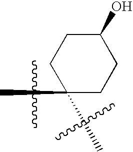 |
| 11 | 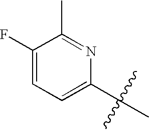 | 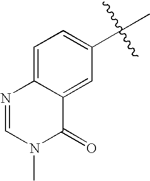 | 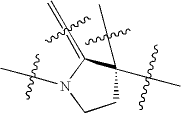 | 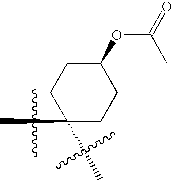 |
| 12 | 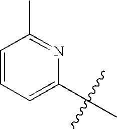 | 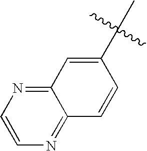 | 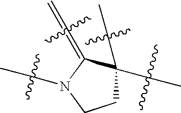 | 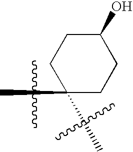 |
| 13 | 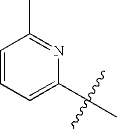 | 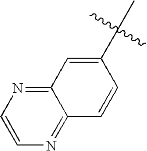 | 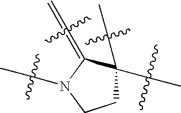 | 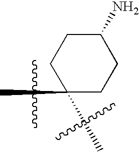 |
| 14 | 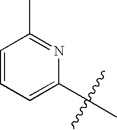 | 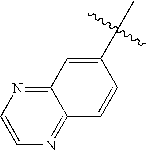 | 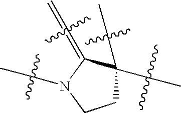 | 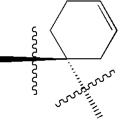 |
| 15-1 | 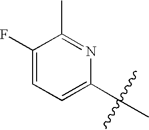 | 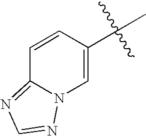 | 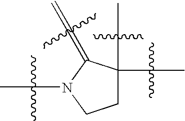 | 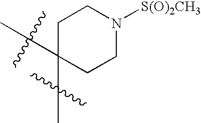 |
| 15-2 | 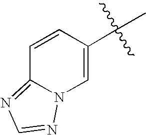 | 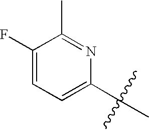 | 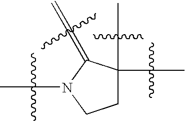 | 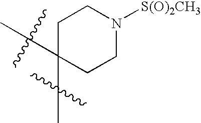 |
| 16 | 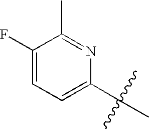 | 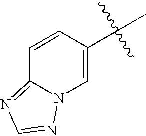 | 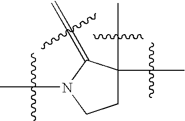 | 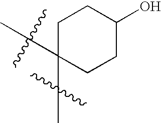 |
| 17 | 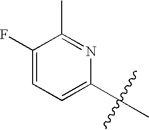 | 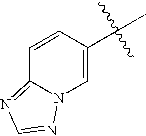 | 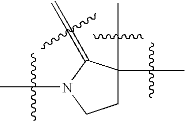 | 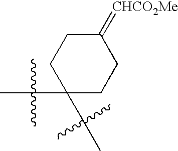 |
| 18-1 | 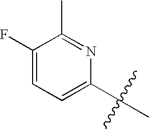 | 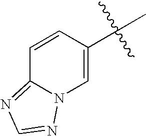 | 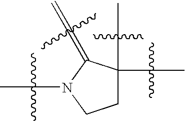 | 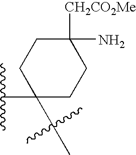 |
| 18-2 | 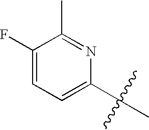 | 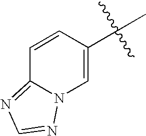 | 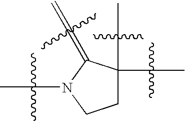 | 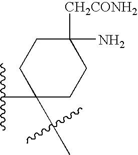 |
| 19 | 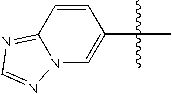 | 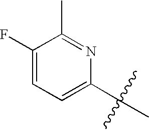 | 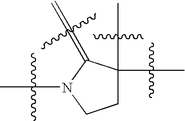 | 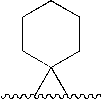 |
| 20 | 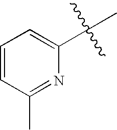 | 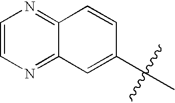 | 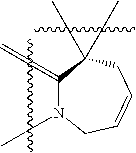 | 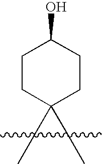 |
| 21 | 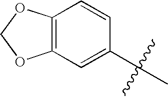 | 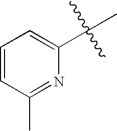 | 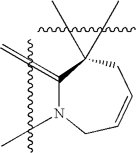 | 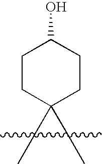 |
| 22 | 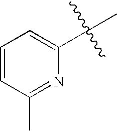 | 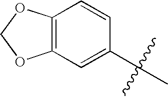 | 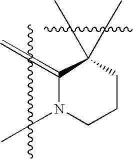 | 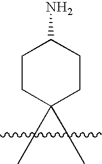 |
| 23 | 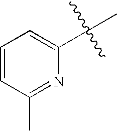 | 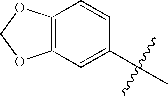 | 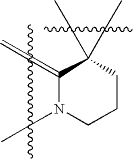 | 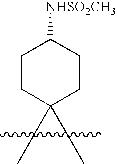 |
| 24 | 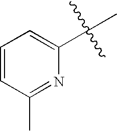 | 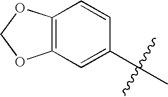 | 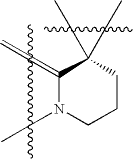 | 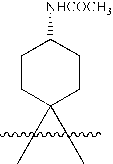 |
| 25 | 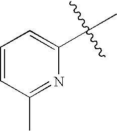 | 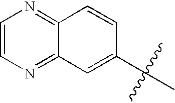 | 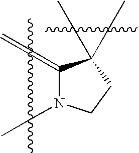 | 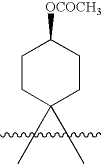 |
| 25a | 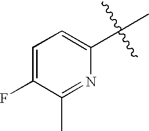 | 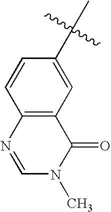 | 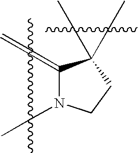 | 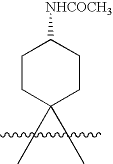 |
| 26 | 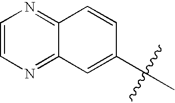 | 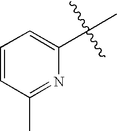 | 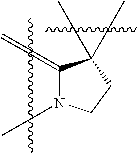 | 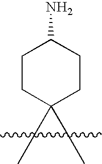 |
| 27 | 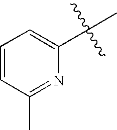 | 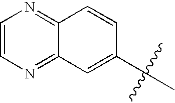 | 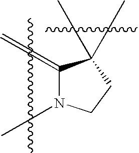 | 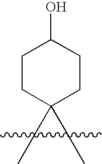 |
| 28 | 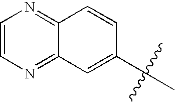 | 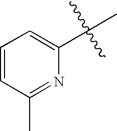 | 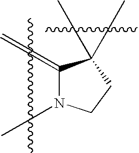 | 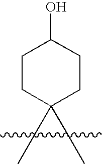 |
| 29 | 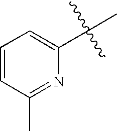 | 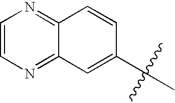 | 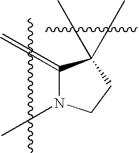 | 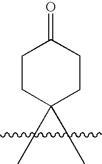 |
| 30 | 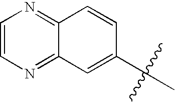 | 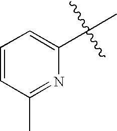 | 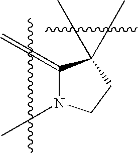 | 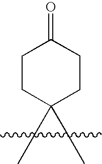 |
| 31 | 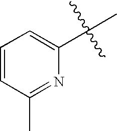 | 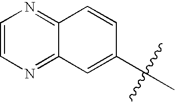 | 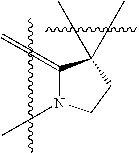 | 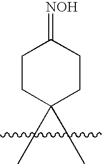 |
| 32 | 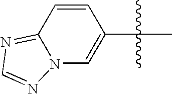 | 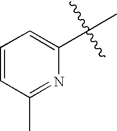 | 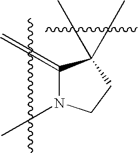 | 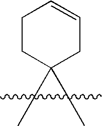 |
| 33 | 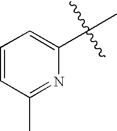 | 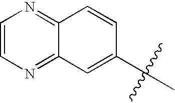 | 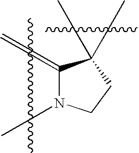 | 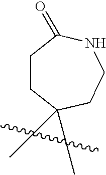 |
| 34 | 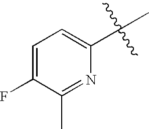 | 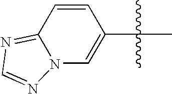 | 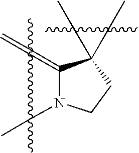 | 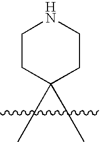 |
| 35 | 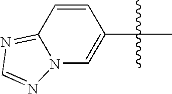 | 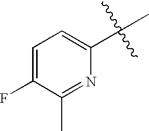 | 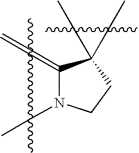 | 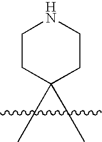 |
| 36 | 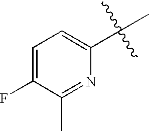 | 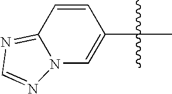 | 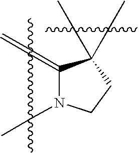 | 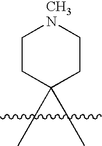 |
| 37 | 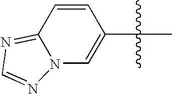 | 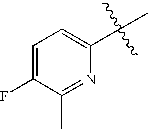 | 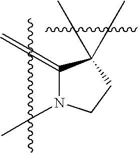 | 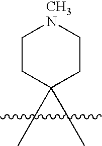 |
| 38 | 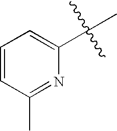 | 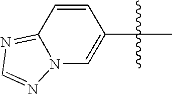 | 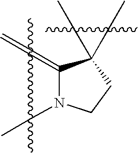 | 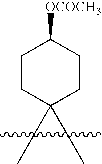 |
| 39 | 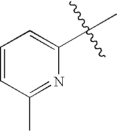 | 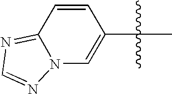 | 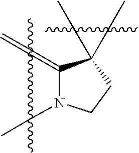 | 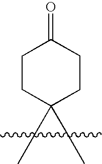 |
| 40 | 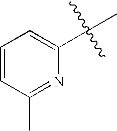 | 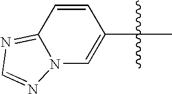 | 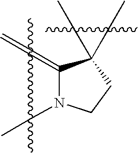 | 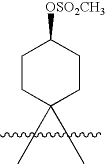 |
| 41 | 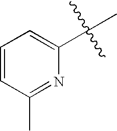 | 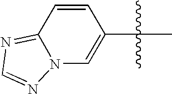 | 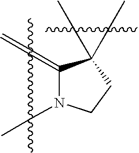 | 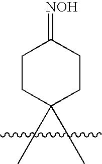 |
| 42 | 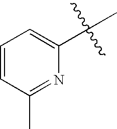 | 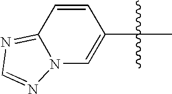 | 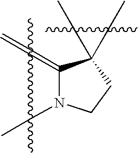 | 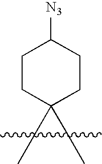 |
| 43 | 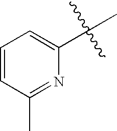 | 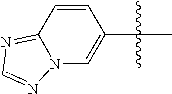 | 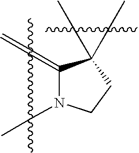 | 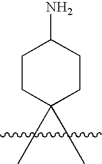 |
| 44 | 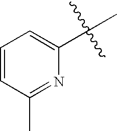 | 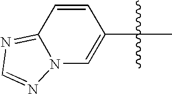 | 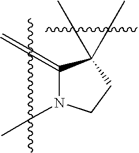 | 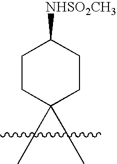 |
| 45 | 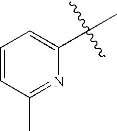 | 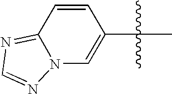 | 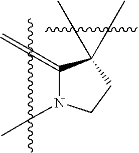 | 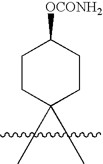 |
| 46 | 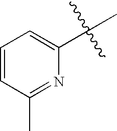 | 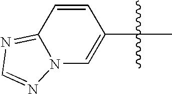 | 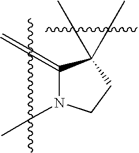 | 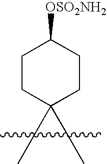 |
| 47 | 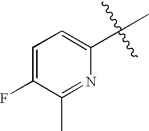 | 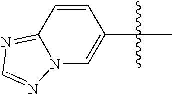 | 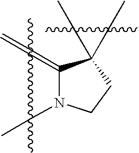 | 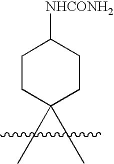 |
| 48 | 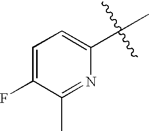 | 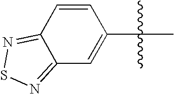 | 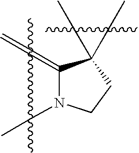 | 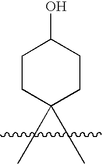 |
| 49 | 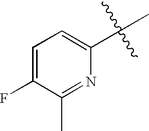 | 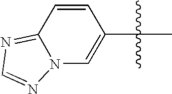 | 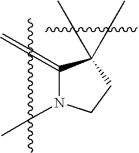 | 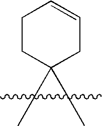 |
| 50 | 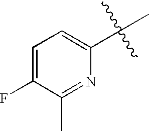 | 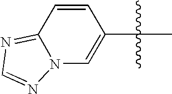 | 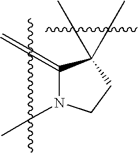 | 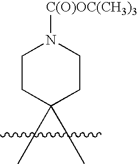 |
| 51 | 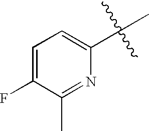 | 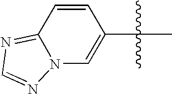 | 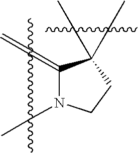 | 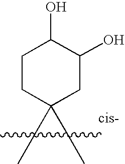 |
| 52 | 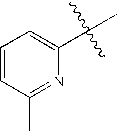 | 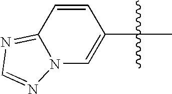 | 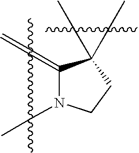 | 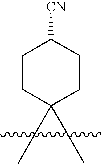 |
| 53 | 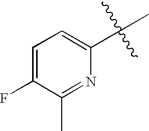 | 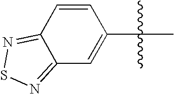 | 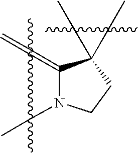 | 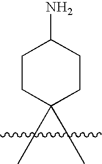 |
| 54 | 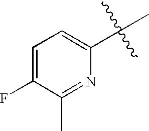 | 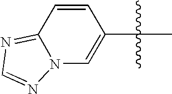 | 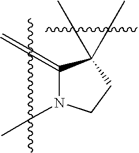 | 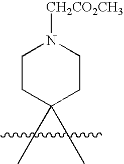 |
| 55 | 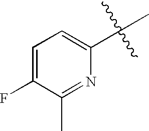 | 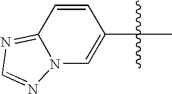 | 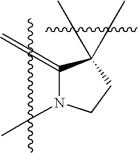 | 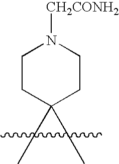 |
| 56 | 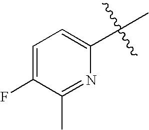 | 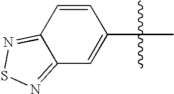 | 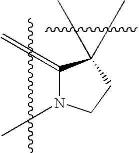 | 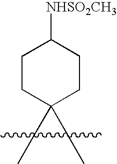 |
| 57 | 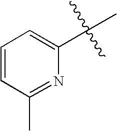 | 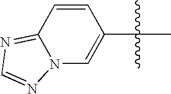 | 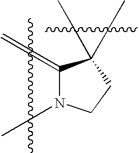 | 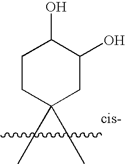 |
| 58 | 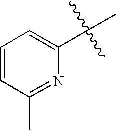 | 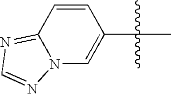 | 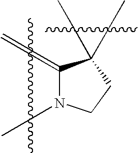 | 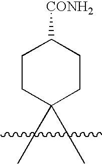 |
| 59 | 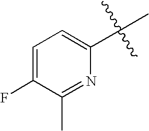 | 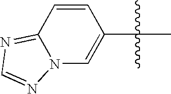 | 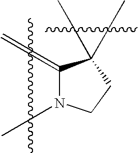 | 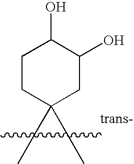 |
| 60 | 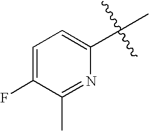 | 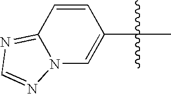 | 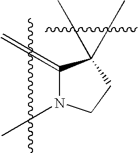 | 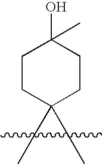 |
| 61 | 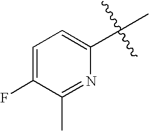 | 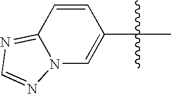 | 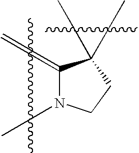 | 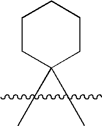 |
| 62 | 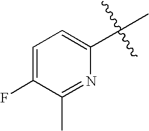 | 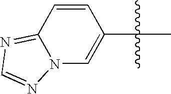 | 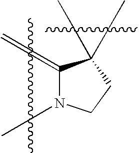 | 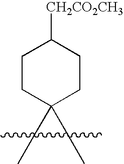 |
| 63 | 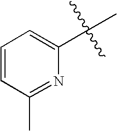 | 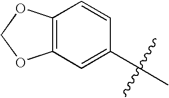 | 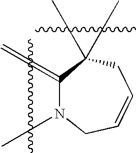 | 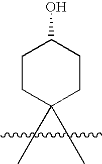 |
| 64 | 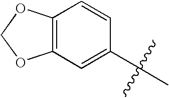 | 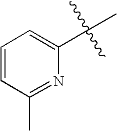 | 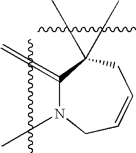 | 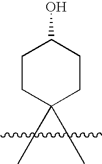 |
| 66 | 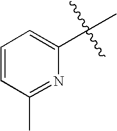 | 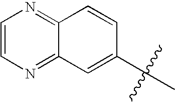 | 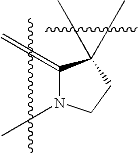 | 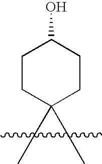 |
| 67 | 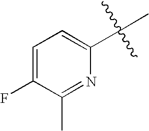 | 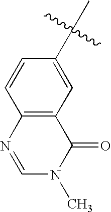 | 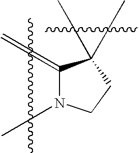 | 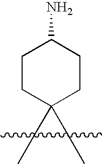 |
| 68 | 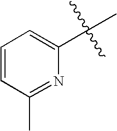 | 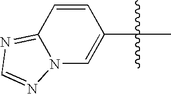 | 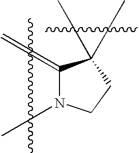 | 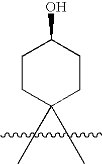 |
| 69 | 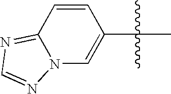 | 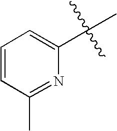 | 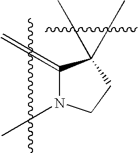 | 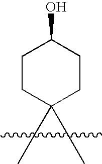 |
| 70 | 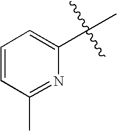 | 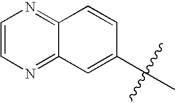 | 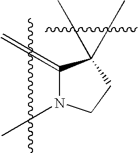 | 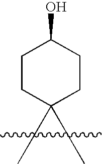 |
| 71 | 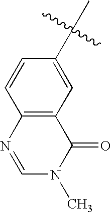 | 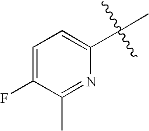 | 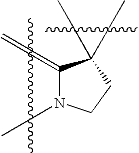 | 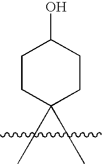 |
| 72 | 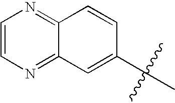 | 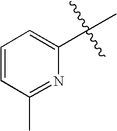 | 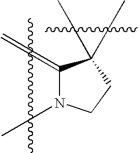 | 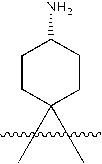 |
| 73 | 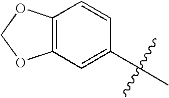 | 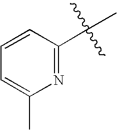 | 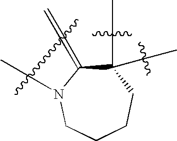 | 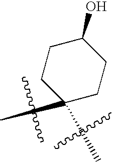 |
| 74 | 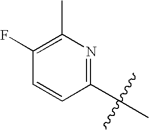 | 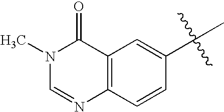 | 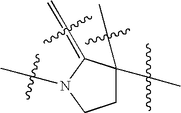 | 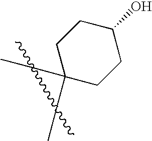 |
| 75 | 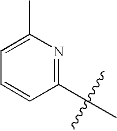 | 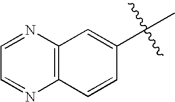 | 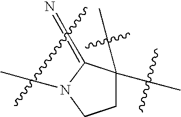 | 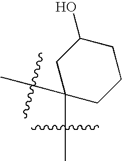 |
| 76 | 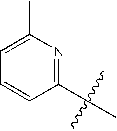 | 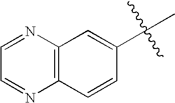 | 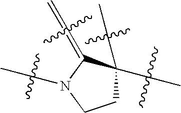 | 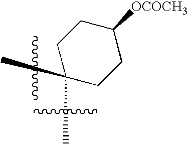 |
| 77 | 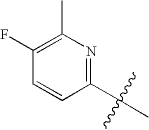 | 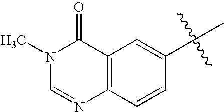 | 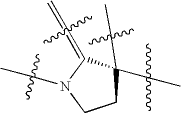 | 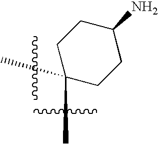 |
| 78 | 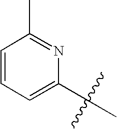 | 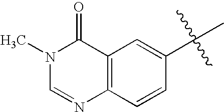 | 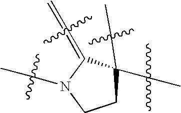 | 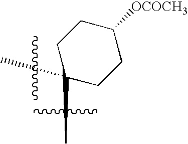 |
| 79 | 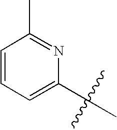 | 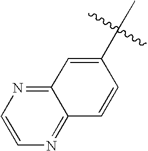 | 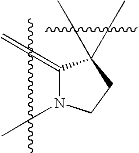 | 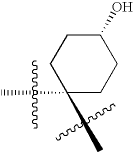 |
| 80 | 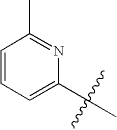 | 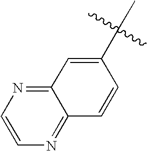 | 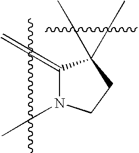 | 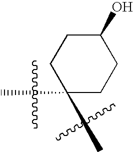 |
| 81 | 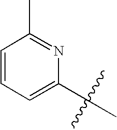 | 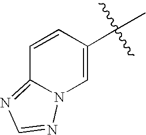 | 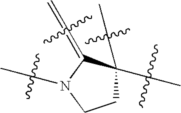 | 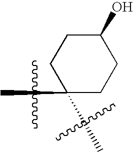 |
| 82 | 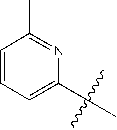 | 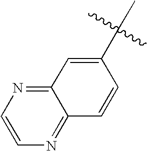 | 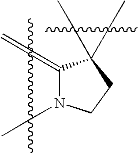 | 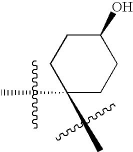 |
| 83 | 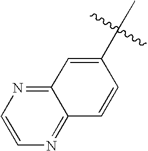 | 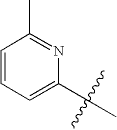 | 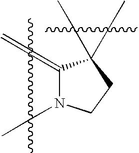 | 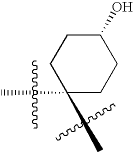 |
| 84 | 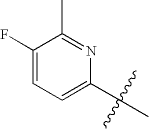 | 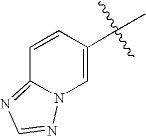 | 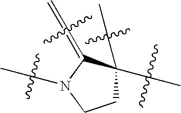 | 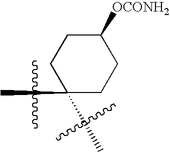 |
| 85 | 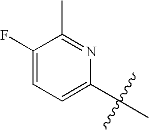 | 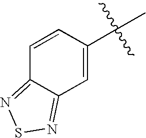 | 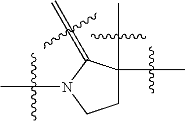 | 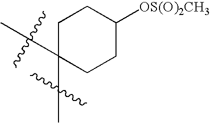 |
| 86 | 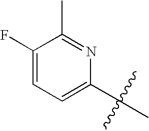 | 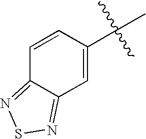 | 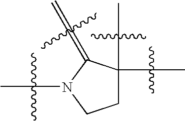 | 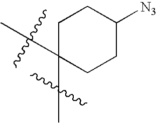 |
| 87 | 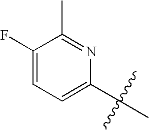 | 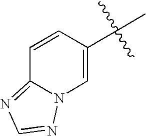 | 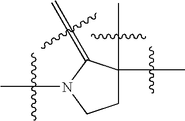 | 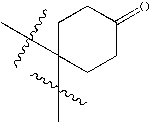 |
| 88 | 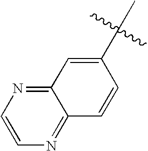 | 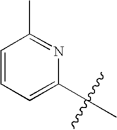 | 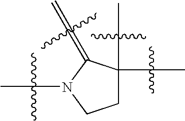 | 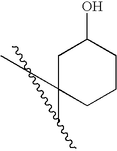 |
| 89 | 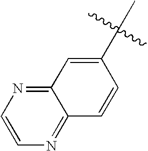 | 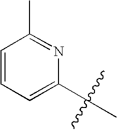 | 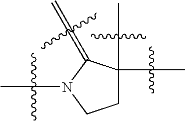 | 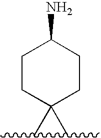 |
| 90 | 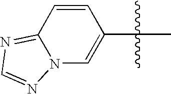 | 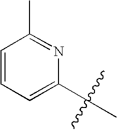 | 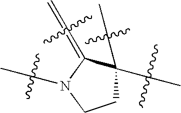 | 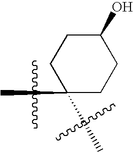 |
| 91 | 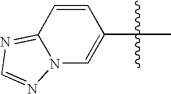 | 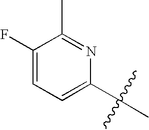 | 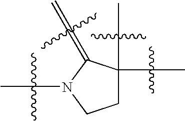 | 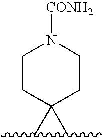 |
| 92 | 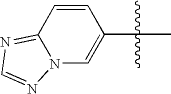 | 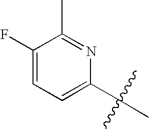 | 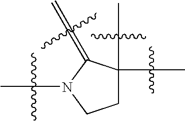 | 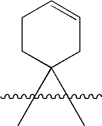 |
| 93 | 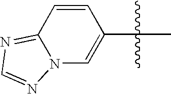 | 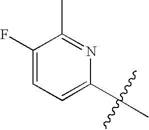 | 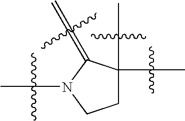 | 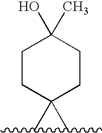 |

Another aspect of the present invention relates to a pharmaceutical composition that includes any of the compounds described above and a pharmaceutically acceptable carrier.

Yet another aspect of the present invention relates to a method of inhibiting the TGFβ signaling pathway in a subject, which includes administering to said subject an effective amount of any of the compounds described above. Still another aspect of the present invention relates to a method of inhibiting the TGFβ type I receptor in a cell, which includes contacting said cell with an effective amount of any of the compounds described above. Yet still another aspect of the present invention relates to a method of reducing the accumulation of excess extracellular matrix induced by TGFβ in a subject, which includes administering to said subject an effective amount of any of the compounds described. Yet still a further aspect of the present invention relates to a method of inhibiting metastasis of tumor cells in a subject, which includes administering to said subject an effective amount of any of the compounds described.

A further aspect of the present invention relates to a method of treating or preventing fibrotic condition in a subject, which includes administering to said subject an effective amount of any of the compounds described above. Examples of such a fibrotic condition include mesothelioma, acute respiratory distress syndrome (ARDS), atherosclerosis, scleroderma, keloids, glomerulonephritis, diabetic nephropathy, lupus nephritis, hypertension-induced nephropathy, cholangitis, restenosis, ocular scarring, corneal scarring, hepatic fibrosis, liver cirrhosis, cirrhosis due to fatty liver disease (alcoholic and nonalcoholic steatosis), biliary fibrosis, pulmonary fibrosis (e.g., idiopathic pulmonary fibrosis), renal fibrosis, sarcoidosis, acute lung injury, drug-induced lung injury, spinal cord injury, central nervous system scarring, systemic lupus erythematosus, Wegener's granulomatosis, cardiac fibrosis, post-infarction cardiac fibrosis, post-surgical fibrosis, connective tissue disease, radiation therapy-induced fibrosis, chemotherapy-induced fibrosis, transplant arteriopathy, fibrosclerosis, fibrotic cancers, fibroids, fibroma, fibroadenomas, and fibrosarcomas.

Also within the scope of the present invention is a method of treating carcinomas mediated by an overexpression of TGFβ, which includes administering to a subject in need of such treatment an effective amount of any of the compounds described above. Examples of the carcinomas include carcinomas of the lung, breast, liver, biliary tract, gastrointestinal tract, head and neck, pancreas, prostate, and cervix, multiple myeloma, melanoma, glioma and glioblastomas.

Still within the scope of the present invention is a method of treating or preventing restinosis, vascular disease, or hypertension by administering to a subject in need thereof any of the compounds described above. Examples of the restinosis include coronary restenosis, peripheral restenosis, and carotid restenosis; examples of the vascular disease include intimal thickening, vascular remodeling, and organ transplant-related vascular disease; and examples of the hypertension include primary and secondary hypertension, systolic hypertension, pulmonary hypertension, and hypertension-induced vascular remodeling.

### Synthesis of Compounds of Formula (I)

Compounds of formula (I), i.e., compounds of this invention, may be prepared by a number of known methods from commercially available or known starting materials. For instance, compounds of formula (I) may be prepared by the generic scheme shown below.

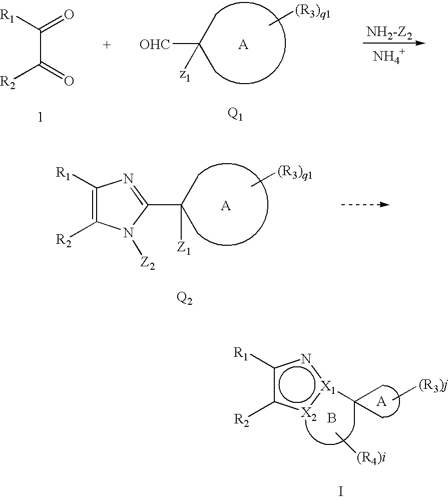

In this scheme, a diketone (1) reacts with a substituted cyclic carboxaldehyde (Q₁) in the presence of the amine NH₂—Z₂ to give an imidazole (Q₂). Further modifications of Z₁ and Z₂ in the imidazole (Q₂), followed by cyclization, provide compounds of formula (I). Z₁ and Z₂ each represent moieties which can be further manipulated to provide functionality suitable for cyclization to provide compounds of formula (I). Suitable moieties for Z₁ and Z₂ include, for example, olefins and protected hydroxyalkyl. When Z₂ is H for example, then suitable functionality in Z₁ includes, for example, hydroxyalkyl, alkylhalide, alkyl bromide, and alkylsulfonate. In a further example, when both Z₁ and Z₂ contain an alkenyl functionality, cyclization can be achieved by a metathesis reaction. When at least one of R₃ or R₄ is a suitable functional group, further modifications can be made as known in the art to provide additional examples of the invention. Further examples of this general scheme are provided below.

In one method, compounds of formula (I) wherein Ring B is a seven membered ring can be prepared according to Scheme 1.

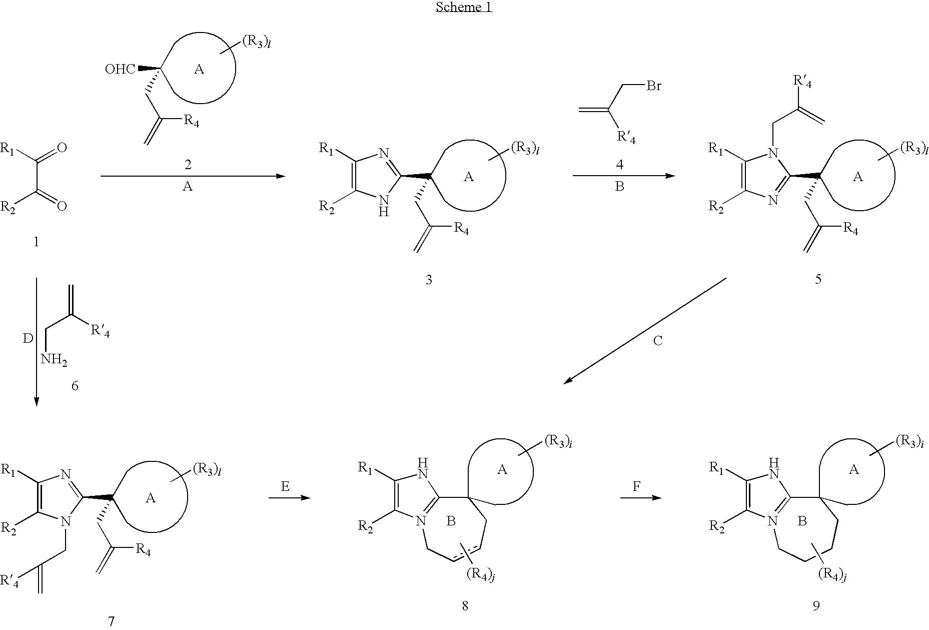

In Scheme 1, R₄ is shown with a specific attachment. It will be recognized that R₄ may have alternative attachments to provide variations in the compounds of the invention. In Step A, a diketone of formula 1 is reacted with an allyl-aldehyde of formula 2 in the presence of an ammonium salt and an organic acid in a suitable solvent to provide the imidazole of formula 3. The diketones 1 are commercially available or may be prepared according to known procedures (see, e.g., U.S. Pat. No. 6,465,493 and U.S. Publication No. 2004/0110797). Suitable ammonium salts include, but are not limited to, ammonium acetate and ammonium chloride. Examples of suitable solvents include dimethoxyethane, methyl-t-butyl ether, dioxane, methanol, ethanol, acetic acid, and dimethylformamide.

In step B, the imidazole of formula 3 is reacted with an allylhalide 4 in the presence of a base in an appropriate solvent to give an alkylated di-allylimidazole imidiazole of formula 5. Suitable bases include, but are not limited to, carbonates such as cesium carbonate, potassium carbonate and the like or a tertiary amine such as diisopropylethyl amine, pyridine and the like. Suitable solvents for this reaction include, e.g., dimethylformamide, N-methylpyrrolidone and sulfolane. The di-allylimidazole of formula 5 is subjected to a metathesis reaction using a ruthenium catalyst to give an imidazole of structure 9 (Grubb's reaction, see, e.g., Grubbs, et al., J. Org. Chem., 1997, 62: 7310; Grubbs et al., J. Amer. Chem. Soc., 2003, 125: 11360; Martin et al., Chem. Rev., 2004, 104: 2199; McReynolds et al., Chem. Rev., 2004, 104: 2239; McDonald et al., J. Am. Chem. Soc. 2004, 126: 2495; J. Am. Chem. Soc., 2000, 122: 8168; Georg et al., Tetrahedron Lett., 2004, 45: 5309; and U.S. Pat. Nos. 5,831,108 and 6,111,121). Examples of suitable ruthenium catalysts include, but are not limited to, benzylidene-bis(tricyclohexylphosphine)dichlororuthenium (Grubbs 1^{st} catalyst), 1,3-bis-(2,4,6-trimethylphenyl)-2-(imidazolidinylidene)dichloro(phenylmethylene)-(tricyclohexylphosphine)ruthenium, and 1,3-(bis(mesityl)-2-imidazolidinylidene)dichloro-(o-isopropoxyphenylmethylene)ruthenium. Examples of suitable solvents for this reaction include methylenechloride, ethylenedichloride, methanol, ethanol, dimethylformamide, and tetrahydrofuran.

Compounds of structure 7 may be reduced to give compounds of structure 8 under olefin hydrogenation conditions known in the art. Further modifications of structures 7 or 8 are readily recognized and exemplified below.

To prepare compounds of this invention in which Ring B is a six-membered ring, the methods illustrated in scheme 2 may be utilized.

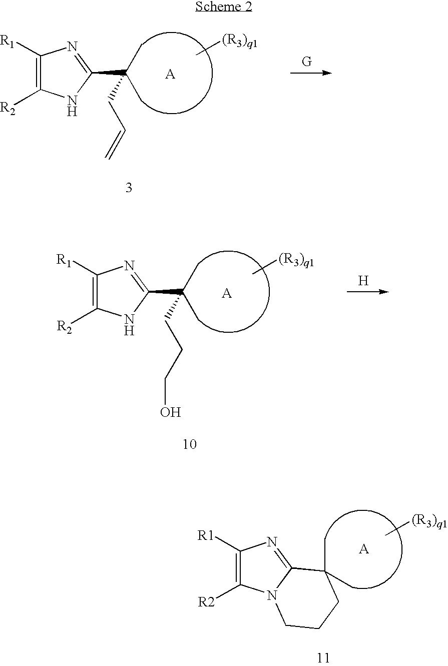

In one method wherein Ring B is a six-membered carbocycle, an allyl compound of structure 3 is reacted with a borane hydride, followed by oxidation with an oxidizing agent to provide an alcohol of structure 10 under conditions known in the art (see, e.g., H. C. Brown, Hydroboration, W. A. Benjamin, New York, 1962). Subsequent cyclization of the alcohol (10) to the six-membered Ring B compound of the invention 7 (step H) may be achieved be contacting a compound of formula 4 with iodine in the presence of a phosphine and a base. Alternatively, cyclization can be achieved by contacting an alcohol 10 with an azodicarboxylate, such as diethylazodicarboxylate or diisopropylazodicarboylate, in the presence of a tertiary phosphine such as triphenylphosphine. Suitable borane hydrides include, for example, diborane and 9-borobicyclo[3.3.1]nonane(9-BBN). Suitable oxidizing agents include, for example, hydrogen peroxide and m-chloroperbenzoic acid. Examples of suitable phosphines include tri-aryl phosphines such as triphenylphosphine. Suitable mild bases include, for example, imidazole, triethylamine, di-isopropylethyl amine, and diazabicycloundecane.

An alternative method for the preparation of compounds of the invention is illustrated in Scheme 3.

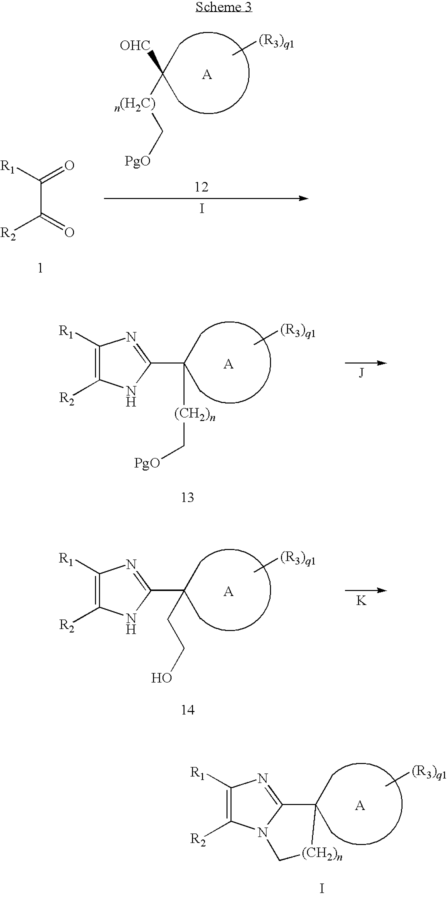

In Scheme 3, an imidazole product (13) of step 1 is prepared by reaction of an aldehyde (12) (n=1-3) with a diketone (1) under conditions as previously described in Step A. Removal of the protecting group (Pg) is followed by ring closure (step K) is achieved as previously described for step H. The starting material diketone (1) shown in the generic scheme and Schemes 1-3 either are commercially available or may be prepared according to known procedures (see, e.g., U.S. Pat. No. 6,465,493 and U.S. Publication No. 2004/0110797).

Shown below in Scheme 4 is another method for preparing some compounds of this invention that include a spiro-ring system fused with a pyrazole core.

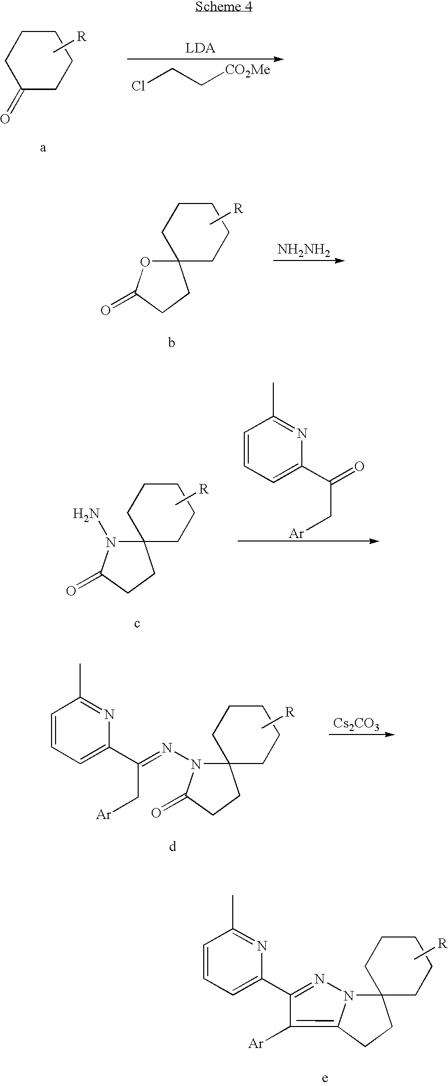

An optionally substituted cycloketone (e.g., cyclohexanone, as compound a shown in Scheme 4) is treated with an ester substituted with halo (e.g., methyl ester of 3-chloropropanoic acid), e.g., in the presence of LDA (diisopropylamide), to give an oxaspiro compound (e.g., 1-oxaspiro[4.5]decan-2-one, as compound b shown in Scheme 4). The oxaspiro compound is then treated with hydrazine to give an amino-substituted azaspirocyclonone (e.g., 1-amino-1-azaspiro[4.5]decan-2-one, as compound c shown in Scheme 4). See, e.g., R. D. Miller, et al., J. Amer. Chem. Soc., 1984, 106, 1508. The azaspirocyclonone compound can then react with pyridinyl ketone (e.g., 1-(6-methylpyridin-2-yl)-2-phenylethanone,) to give an azaspirocyclonone substituted with an imino group (e.g., (E)-1-(1-(6-methylpyridin-2-yl)-2-arylethylideneamino)-1-azaspiro[4.5]decan-2-one, as compound d shown in Scheme 4). Synthesis of a pyridinyl ketone can be found, e.g., in W. C. Lee et al., PCT publication WO 03/087304 A2, the content of which is incorporated herein by reference in its entirety. The resulting N-substituted azaspirocyclonone can then be treated with cesium carbonate to give a pyrazole-fused spiro compound (e.g., 2′-(6-methylpyridin-2-yl)-3′-aryl-4′,5′-dihydrospiro[cyclohexane-1,6′-pyrrolo[1,2-b]pyrazole], as compound e shown in Scheme 4). See, e.g., Beight, D. W., et al., PCT publication WO 2004048382 A1, the content of which is also incorporated herein by reference in its entirety.

As previously mentioned, modification of substituents on Rings A or B provide additional examples of the compounds of this invention (see, e.g, WO 03/087304). To illustrate, compounds of this invention wherein Ring A contains a heteroatom may be prepared by any of the above Schemes utilizing a starting material of structure 16

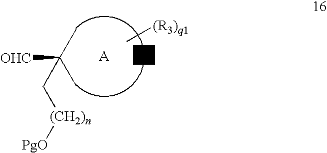

(wherein X is S, O, or NPg, each Pg is independently a protecting group, and n is 1, 2, or 3) to give compounds of structure 17

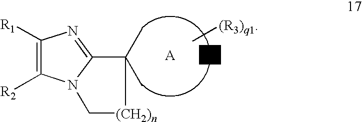

When X is NPg, the protecting group may be removed and further modifications of the resultant NH may be made as are known in the art, e.g. preparation of amide, carbamate, sulfonamide, urea, and alkyl or aralkyl moieties.

When one of R₃ is hydroxy or a protected hydroxy, further modifications can be achieved. For example, when R₃ is a protected hydroxy, further modifications can be made by removal of the protecting group to provide the corresponding alcohol which can be converted to alcohol derivatives such esters, thioesters, carbamates, halides, nitriles, alkyl ethers, aryl ethers, and the like. The alcohol may also be converted to the corresponding amine, ketone or olefin utilizing methods known in the art. Further modifications may provide, for example, a substituted amine, a cis or trans 1,2-glycol or a homologated lactam using known methodology. In another example, R₃ may be —CH₂OPg. Removal of the protecting group Pg provides an alcohol which can be further converted to alcohol derivatives as described above. Oxidation of the primary alcohol can further provide an aldehyde or a carboxylic acid which in turn can be further modified or derivatized. In another embodiment, when two R₃ on the same atom form a ketone, a Wittig type reaction may be performed to produce, for example, an unsaturated ester. Further modifications of the unsaturated ester can include, for example, conversion to the amide, reduction of the double bond or Michael addition of nucleophiles.

### Uses of Compounds of Formula (1)

### A. Indications

As discussed above, hyperactivity of the TGFβ family signaling pathways can result in excess deposition of extracellular matrix and increased inflammatory responses, which can then lead to fibrosis in tissues and organs (e.g., lung, kidney, and liver) and ultimately result in organ failure. See, e.g., Bordek, W. A. and Ruoslahti, E. J., Clin. Invest, 90:1-7 (1992) and Border, W. A. and Noble, N. A., N. Engl. J. Med., 331: 1286-1292 (1994). Studies have shown that the expression of TGFβ and/or activin mRNA and the level of TGFβ and/or activin are increased in patients suffering from various fibrotic disorders, e.g., fibrotic kidney diseases, alcohol-induced and autoimmune hepatic fibrosis, myelofibrosis, bleomycin-induced pulmonary fibrosis, and idiopathic pulmonary fibrosis.

Compounds of formula (I), which are antagonists of the TGFβ family type I receptors Alk5 and/or Alk4, and inhibit TGFβ and/or activin signaling pathway, are therefore useful for treating and/or preventing fibrotic disorders or diseases mediated by an increased level of TGFβ and/or activin activity. As used herein, a compound of formula (I) inhibits the TGFβ family signaling pathway when it binds (e.g., with an IC₅₀ value of less than 10 μM, such as less than 1 μM; and for example, less than 5 nM) to a receptor of the pathway (e.g., Alk5 and/or Alk4), thereby competing with the endogenous ligand(s) or substrate(s) for binding site(s) on the receptor and reducing the ability of the receptor to transduce an intracellular signal in response to the endogenous ligand or substrate binding. The aforementioned disorders or diseases include any condition (a) marked by the presence of an abnormally high level of TGFβ and/or activin; and/or (b) an, excess accumulation of extracellular matrix; and/or (c) an increased number and synthetic activity of myofibroblasts. These disorders or diseases include, but are not limited to, fibrotic conditions such as acute respiratory distress syndrome (ARDS), atherosclerosis, keloids, sarcoidosis, scleroderma, glomerulonephritis, diabetic nephropathy, lupus nephritis, hypertension-induced nephropathy, ocular or corneal scarring, alimentary track or gastrointestinal fibrosis, renal fibrosis, hepatic or biliary fibrosis, acute lung injury, pulmonary fibrosis (such as radiation-induced pulmonary fibrosis or idiopathic pulmonary fibrosis), post-infarction cardiac fibrosis, fibrosclerosis, fibrotic cancers, fibroids, fibroma, fibroadenomas, fibrosarcomas, spinal cord injury, systemic lupus erythematosus, and Wegener's granulomatosis. Other fibrotic conditions for which preventive treatment with compounds of formula (I) can have therapeutic utility include radiation therapy-induced fibrosis, chemotherapy-induced fibrosis, and surgically induced scarring including surgical adhesions, laminectomy, and coronary restenosis.

Increased TGFβ activity is also found to manifest in patients with progressive cancers. Studies have shown that in late stages of various cancers, both the tumor cells and the stromal cells within the tumors generally overexpress TGFβ. This leads to stimulation of angiogenesis and cell motility, suppression of the immune system, and increased interaction of tumor cells with the extracellular matrix. See, e.g., Hojo, M. et al., Nature, 397: 530-534 (1999). As a result, the tumor cells become more invasive and metastasize to distant organs. See, e.g., Maehara, Y. et al., J. Clin. Oncol., 17: 607-614 (1999) and Picon, A. et al., Cancer EpidemioL Biomarkers Prev., 7: 497-504 (1998). Thus, compounds of formula (I), which are antagonists of the TGFβ type I receptor and inhibit TGFβ signaling pathways, are also useful for treating and/or preventing various late stage cancers which overexpress TGFβ. Such late stage cancers include carcinomas of the lung, breast, liver, biliary tract, gastrointestinal tract, head and neck, pancreas, prostate, cervix as well as multiple myeloma, melanoma, glioma, and glioblastomas.

Importantly, it should be pointed out that because of the chronic, and in some cases localized, nature of disorders or diseases mediated by overexpression of TGFβ and/or activin (e.g., fibrosis or cancers), small molecule treatments (such as treatment disclosed in the present invention) are favored for long-term treatment.

Not only are compounds of formula (I) useful in treating disorders or diseases mediated by high levels of TGFβ and/or activin activity, these compounds can also be used to prevent the same disorders or diseases. It is known that polymorphisms leading to increased TGFβ and/or activin production have been associated with fibrosis and hypertension. Indeed, high serum TGFβ levels are correlated with the development of fibrosis in patients with breast cancer who have received radiation therapy, chronic graft-versus-host-disease, idiopathic interstitial pneumonitis, veno-occlusive disease in transplant recipients, and peritoneal fibrosis in patients undergoing continuous ambulatory peritoneal dialysis. Thus, the levels of TGFβ and/or activin in serum and of TGFβ and/or activin mRNA in tissue can be measured and used as diagnostic or prognostic markers for disorders or diseases mediated by overexpression of TGFβ and/or activin, and polymorphisms in the gene for TGFβ that determine the production of TGFβ and/or activin can also be used in predicting susceptibility to disorders or diseases. See, e.g., Blobe, G. C. et al., N. Engl. J. Med., 342(18): 1350-1358 (2000); Matsuse, T. et al., Am. J. Respir. Cell Mol. Biol., 13: 17-24 (1995); Inoue, S. et al., Biochem. Biophys. Res. Comm., 205: 441-448 (1994); Matsuse, T. et al, Am. J. Pathol., 148: 707-713 (1996); De Bleser et al., Hepatology, 26: 905-912 (1997); Pawlowski, J. E. et al., J. Clin. Invest., 100: 639-648 (1997); and Sugiyama, M. et al., Gastroenterology, 114: 550-558 (1998).

Additionally, compounds of this invention are also effective at treating, preventing, or reducing intimal thickening, vascular remodeling, restenosis (e.g., coronary, peripheral, and carotid restenosis), vascular diseases (e.g., organ transplant-related, cardiac, and renal diseases), and hypertension (e.g., primary and secondary, systolic, pulmonary, and hypertension-induced vascular remodeling resulting in target organ damage).

Without wishing to be bound by any particular theory, one possible explanation for the efficacy of the compounds of this invention may be their inhibitory effect on the TGFβ and activin pathways.

The pathological activation of the TGFβ and activin pathway plays a critical role in the progression of fibrotic diseases. The critical serine-threonine kinase in the TGFβ type I receptor (TGFβRI) and the activin type I receptor (Alk4) are attractive targets for blockade of the TGFβ pathway for several important reasons. TGFβRI kinase activity is required for TGFβ signaling as is Alk4 for activin signaling. Kinases have proven to be useful targets for development of small molecule drugs. There is a good structural understanding of the TGFβRI kinase domain allowing the use of structure-based drug discovery and design to aid in the development of inhibitors.

TGFβ or activin-mediated pathological changes in vascular flow and tone are often the cause of morbidity and mortality in a number of diseases (see, e.g., Gibbons G. H. and Dzau V. J., N. Eng. J. Med., 330:1431-1438 (1994)). Typically, the initial response of the vasculature to injury is an infiltration of adventitial inflammatory cells and induction of activated myofibroblasts or smooth muscle cells (referred to as myofibroblasts from hereon). TGFβ is initially produced by infiltrating inflammatory cells and activates myofibroblasts or smooth muscle cells. These activated myofibroblasts can also secrete TGFβ as well as respond to it. Within the first few days following injury, myofibroblasts secreting TGFβ migrate from the various layers of the vascular wall towards the lumen where they undergo proliferation and extracellular matrix secretion resulting in intimal thickening. Additionally, TGFβ induces activated myofibroblasts to contract which results in lumenal narrowing. These vascular remodeling processes, intimal thickening and vascular contraction, restrict blood flow to the tissues supported by the effected vasculature and result in tissue damage. Activin is also produced in response to injury and shows very similar actions in inducing activated myofibroblasts or activated smooth muscle cells intimal thickening and vascular remodeling. See, for example, Pawlowski et al., J. Clin. Invest., 100: 639-648 (1997); Woodruff T. K., Biochem. Pharmacol., 55: 953-963 (1998); Molloy et al., J. Endocrinol., 161(2): 179-85 (1999); and Harada, K. et al., J. Clin Endocrinol Metab., 81(6): 2125-30 (1996).

In coronary, peripheral or carotid artery disease, balloon angioplasty or stent placement is used to increase lumen size and blood flow. However, the physical damage created by stretching the vessel wall causes injury to the vessel wall tissue. TGFβ elevation following injury induces myofibroblasts in 2-5 days and frequently results in restenosis within 6 months of balloon angioplasty or within a few years of stent placement in human patients. Following balloon angioplasty, both intimal thickening and vascular remodeling due to myofibroblast contraction, cause narrowing of the lumen and decreased blood flow. Stent placement physically prevents remodeling, but hyperplasia and extracellular matrix deposition by activated myofibroblasts proliferating at the luminal side of the stent result in intimal thickening within the stented vessel and the eventual impairment of blood flow.

The treatment of arterial stenotic disease by surgical grafts, e.g. coronary bypass or other bypass surgery, also can elicit restenosis in the grafted vessel. In particular, vein grafts undergo intimal thickening and vascular remodeling through a similar mechanism involving TGFβ-induced intimal thickening and vascular remodeling. In this case, the injury is either due to the overdistention of the thin-walled vein graft placed into an arterial vascular context or due to anastamotic or ischemic injury during the transplantation of the graft.

The loss of patency in arteriovenous or synthetic bridge graft fistulas is another vascular remodeling response involving increased TGFβ production. See, e.g., Ikegaya, N. et al., J. Am. Soc. Nephrol., 11: 928-35 (2000) and Heine, G. H. et al., Kidney Int., 64: 1101-7 (2003). Loss of fistula patency causes complications for renal dialysis or other treatments requiring chronic access to the circulatory system. See, e.g., Ascher, E., Ann. Vasa Surg. 15: 89-97 (2001). Blockade of TGFβ by TGFβRI inhibitors will beneficial for preventing restenosis and extending arteriovenous fistula patency.

Elevated TGFβ activity is also implicated in chronic allograft vasculopathy in both animals and humans. Vascular injury (e.g., intimal thickening and vascular remodeling) is a characteristic pathology in chronic allograft failure. The fibrotic response in chronic allograft failure initiates in the vasculature of the donor organ. Chronic allograft vasculopathy in allografted hearts often manifests within 5 years of transplantation and is the main cause of death in long term survivors of cardiac transplant. Both early detection of cardiac allograft vasculopathy measured as intimal thickening by intravascular ultrasound as well as the elevation of plasma TGFβ has been suggested as a prognostic marker for late cardiac allograft failure. See, e.g., Mehra, M. R. et al., Am. J. Transplant, 4: 1184 (2004). Cardiac biopsies of grafted hearts also suggest that graft tissue expression of TGFβ correlates significantly to vasculopathy and the number of rejection episodes (see, e.g., Aziz, T. et al., J. Thorac. Cardiovasc. Surg., 119: 700 (2000)). Finally, patients with high-producing TGFβ genotypes are more susceptible to earlier onset cardiac-transplant coronary vasculopathy. See, e.g, Densem, C. G. et al., J. Heart Lung. Transplant, 19: 551 (2000); Aziz, T. et al., J. Thorac. Cardiovasc. Surg., 119: 700 (2000); and Holweg, C. T., Transplantation, 71: 1463 (2001).

Elevation of TGFβ activity can be induced by ischemic, immune and inflammatory responses to the allograft organ. Animal models of acute and chronic renal allograft rejection identify the elevation of TGFβ activity as a significant contributor to graft failure and rejection. See, e.g., Nagano, H. et al., Transplantation, 63: 1101 (1997); Paul, L. C., et al., Am. J. Kidney Dis., 28: 441 (1996); and Shihab, F. S. et al., Kidney Int., 50: 1904 (1996).

Rodent models of chronic allograft nephropathy (CAN) show elevation of TGFβ mRNA and immunostaining. In renal allografts TGFβ immunostaining is strongly positive in interstitial inflammatory andfibrotic cells, but also in blood vessels and glomeruli. In humans, the loss of renal function 1 year post renal allograft correlates with TGFβ staining in the grafted kidney. See, e.g., Cuhaci, B. et al., Transplantation, 68: 785 (1999). Graft biopsies show also that renal dysfunction correlates with chronic vascular remodeling, ie vasculopathy, and the degree of TGFβ expression correlates significantly with chronic vasculopathy. See, e.g., Viklicky, O. et al., Physiol Res., 52:353 (2003).

The use of immunosuppressive agents such as cyclosporine A in organ transplantation has not prevented vasculopathy and chronic allograft nephropathy suggesting non-immune mechanisms are involved in allograft failure. In fact, cyclosporinA and other immunosuppressants have been shown to induce TGFβ expression and may contribute to vasculopathy. See, e.g., Moien-Afshari, F. et al., Pharmacol Ther., 100: 141 (2003) and Jain, S. et al., Transplantation, 69: 1759 (2000).

TGFβ is implicated in chronic allograft rejection in both renal and lung transplants due to the clear TGFβ-related fibrotic pathology of this condition as well as the ability of immune suppressants, especially cyclosporin A, to induce TGFβ. See, e.g., Jain, S. et al., Transplantation, 69: 1759 (2000). TGFβ blockade improved renal function while decreasing collagen deposition, renal TGFβ expression as well as vascular afferent arteriole remodeling in a cyclosporine A-induced renal failure model using an anti-TGFβ monoclonal antibody (see, e.g., Islam, M. et al., Kidney Int., 59: 498 (2001) and Khanna, A. K. et al., Transplantation, 67: 882 (1997). These data are strongly indicative of a causal role for TGFβ in the development and progression of chonic allograft vasculopathy and chronic allograft failure.

Hypertension is a major cause of morbidity and mortality in the U.S. population affecting approximately 1 in every 3 individuals. The effect of hypertension on target organs include increased incidence of cardiac failure, myocardial-infarction, stroke, renal failure, aneurysm, and microvascular hemorrhage. Hypertension-induced damage to the vasculature results in vascular remodeling and intimal thickening which are a major causative factor in many of these morbidities (see, e.g., Weber, W. T., Curr. Opin. Cardiol., 15: 264-72 (2000)). Animal experiments suggest that TGFβ activity is elevated upon induction of hypertension and anti-TGFβ monoclonal antibody blockade of this pathway decreases blood pressure and renal pathology in hypertensive rats (see, e.g., Xu, C. et al., J. Vasc. Surg., 33: 570 (2001) and Dahly, A. J. et al., Am. J. Physiol. Regul. Integr. Comp. Physiol., 283: R757 (2002)). In humans, plasma TGFβ level is elevated in hypertensive individuals compared to normotensive controls and plasma TGFβ level is also higher in hypertensive individuals with manifest target organ disease compared to hypertensive individuals without apparent target organ damage (see, e.g., Derhaschnig, U. et al., Am. J. Hypertens., 15: 207 (2002); and Suthanthiran, M., Proc. Natl. Acad. Sci. USA, 97: 3479 (2000)). There is also evidence suggesting that high TGFβ-producing genotypes of TGFβ are a risk factor for development of hypertension (see, e.g., Lijnen, P. J., Am. J. Hypertens., 16: 604 (2003); and Suthanthiran, M., Proc. Natl. Acad. Sci. USA, 97: 3479 (2000)). Thus the inhibition of the TGFβ pathway may provide an effective therapeutic approach for hypertension or hypertension-induced organ damage.

The vascular injury response in the pulmonary vasculature results in pulmonary hypertension which can lead to overload of the right heart and cardiac failure. See, e.g., Runo, J. R. and Loyd, J. E., Lancet, 361(9368): 1533-44 (2003); Sitbon, O. et al., Prog. Cardiovasc. Dis., 45: 115-28 (2002); and Jeffery, T. K. and Morrell, N. W., Cardiovasc. Dis., 45: 173-202 (2002). Prevention of pulmonary vascular remodeling by TGFβRI inhibitors can be of practical utility in diseases such as primary or secondary pulmonary hypertension. See, e.g., Sitbon, O. et al., Prog Cardiovasc. Dis., 45: 115-28 (2002); and Humbert, M. et al., J. Am. Coll. Cardiol., 43: 13S-24S (2004). Inhibition of the progression of vascular remodeling over time will prevent the progression of pulmonary pathology in these life threatening diseases. Secondary pulmonary hypertension occurs often as a manifestation of scleroderma and is one of the primary causes of morbidity and mortality in scleroderma patients (see, e.g., Denton, C. P. and Black, C. M., Rheum. Dis. Clin. North Am., 29: 335-49 (2003)). Pulmonary hypertension is also a sequalae of mixed connective tissue disease, chronic obstructive pulmonary disease (COPD) and lupus erythematosis (see, e.g., Fagan, K. A. and Badesch, D. B., Prog. Cardiovasc. Dis., 45:225-34 (2002); and Presberg, K. W. and Dincer, H. E., Curr. Opin. Pulm. Med., 9:131-8 (2003)).

Many of the diseases described above involving vascular remodeling are particularly severe in diabetic patients (see, e.g., Reginelli, J. P. and Bhatt, D. L., J. Invasive Cardiol., 14 Suppl E: 2E-10E (2002)). Elevated glucose in diabetes can itself induce TGFβ which leads to the increased vascular remodeling and intimal thickening response to vascular injury (see, e.g., Ziyadeh, F. J., Am. Soc. Nephrol., 15 Suppl 1: S55-7 (2004)). In particular, diabetic patients have significantly higher rates of restenosis, vein graft stenosis, peripheral artery disease, chronic allograft nephropathy and chronic allograft vasculopathy (see, e.g., Reginelli, J. P. and Bhatt D. L., J. Invasive Cardiol. 14 Suppl E: 2E-10E (2002); Eisen, H. and Ross, H., J. Heart Lung Transplant., 23: S207-13 (2004); and Valentine, H., J. Heart Lung Transplant., 23: S187-93 (2004)). Thus, blockade of TGFβ is of particular utility in diabetic patients at risk for hypertension-related organ failure, diabetic nephropathy, restenosis or vein graft stenosis in coronary or peripheral arteries, and chronic failure of allograft organ transplants (see, e.g., Endemann, D. H. et al., Hypertension, 43(2): 399-404 (2004); Ziyadeh, F. J., Am. Soc. Nephrol., 15 Suppl 1: S55-7 (2004); and Jerums, G. et al., Arch Biochem. Biophys., 419: 55-62 (2003)).

TGFβRI and Alk4 antagonists are effective at treating, preventing, or reducing intimal thickening, vascular remodeling, restenosis (e.g., coronary, peripheral, carotid restenosis), vascular diseases (e.g., organ transplant-related, cardiac, and renal), and hypertension (e.g., systolic, pulmonary, and hypertension-induced vascular remodeling resulting in target organ damage). Changes in vascular remodeling and intimal thickening may be qualified by measuring the intimal versus medial vascular thickness.

### B. Administration of Compounds of Formula (I)

As defined above, an effective amount is the amount required to confer a therapeutic effect on the treated patient. For a compound of formula (I), an effective amount can range, for example, from about 1 mg/kg to about 150 mg/kg (e.g., from about 1 mg/kg to about 100 mg/kg). The effective amount may also vary, as recognized by those skilled in the art, dependant on route of administration, excipient usage, and the possibility of co-usage with other therapeutic treatments including use of other therapeutic agents and/or radiation therapy.

Compounds of formula (I) can be administered in any manner suitable for the administration of pharmaceutical compounds, including, but not limited to, pills, tablets, capsules, aerosols, suppositories, liquid formulations for ingestion or injection or for use as eye or ear drops, dietary supplements, and topical preparations. The pharmaceutically acceptable compositions include aqueous solutions of the active agent, in an isotonic saline, 5% glucose or other well-known pharmaceutically acceptable excipient. Solubilizing agents such as cyclodextrins, or other solubilizing agents well-known to those familiar with the art, can be utilized as pharmaceutical excipients for delivery of the therapeutic compounds. As to route of administration, the compositions can be administered orally, intranasally, transdermally, intradermally, vaginally, intraaurally, intraocularly, buccally, rectally, transmucosally, or via inhalation, implantation (e.g., surgically), or intravenous administration. The compositions can be administered to an animal (e.g., a mammal such as a human, non-human primate, horse, dog, cow, pig, sheep, goat, cat, mouse, rat, guinea pig, rabbit, hamster, gerbil, or ferret, or a bird, or a reptile such as a lizard).

In certain embodiments, the compounds of formula (I) can be administered by any method that permits the delivery of the compound to combat vascular injuries. For instance, the compounds of formula (I) can be delivered by any method described above. Additionally, the compounds of formula (I) can be administered by implantation (e.g., surgically) via an implantable device. Examples of implantable devices include, but are not limited to, stents, delivery pumps, vascular filters, and implantable control release compositions. Any implantable device can be used to deliver the compound provided that (i) the device, compound and any pharmaceutical composition including the compound are biocompatible, and (ii) that the device can deliver or release an effective amount of the compound to confer a therapeutic effect on the treated patient.

Delivery of therapeutic agents via stents, delivery pumps (e.g., mini-osmotic pumps), and other implantable devices is known in the art. See, e.g, Hofma, et al., Current Interventional Cardiology Reports, 3: 28-36 (2001), the entire contents of which, including references cited therein, are incorporated herein. Other descriptions of implantable devices, such as stents, can be found in U.S. Pat. Nos. 6,569,195 and 6,322,847; and PCT International Publication Numbers WO04/0044405, WO04/0018228, WO03/0229390, WO03/0228346, WO03/0225450, WO03/0216699, and WO03/0204168, each of which is also incorporated herein in by reference its entirety.

A delivery device, such as stent, includes a compound of formula (I). The compound may be incorporated into or onto the stent using methodologies known in the art. In some embodiments, a stent can include interlocked meshed cables. Each cable can include metal wires for structural support and polyermic wires for delivering the therapeutic agent. The polymeric wire can be dosed by immersing the polymer in a solution of the therapeutic agent. Alternatively, the therapeutic agent can be embedded in the polymeric wire during the formation of the wire from polymeric precursor solutions. In other embodiments, stents or implatable devices can be coated with polymeric coatings that include the therapeutic agent. The polymeric coating can be designed to control the release rate of the therapeutic agent.

Controlled release of therapeutic agents can utilize various technologies. Devices are known having a monolithic layer or coating incorporating a heterogeneous solution and/or dispersion of an active agent in a polymeric substance, where the diffusion of the agent is rate limiting, as the agent diffuses through the polymer to the polymer-fluid interface and is released into the surrounding fluid. In some devices, a soluble substance is also dissolved or dispersed in the polymeric material, such that additional pores or channels are left after the material dissolves. A matrix device is generally diffusion limited as well, but with the channels or other internal geometry of the device also playing a role in releasing the agent to the fluid. The channels can be pre-existing channels or channels left behind by released agent or other soluble substances.

Erodible or degradable devices typically have the active agent physically immobilized in the polymer. The active agent can be dissolved and/or dispersed throughout the polymeric material. The polymeric material is often hydrolytically degraded over time through hydrolysis of labile bonds, allowing the polymer to erode into the fluid, releasing the active agent into the fluid. Hydrophilic polymers have a generally faster rate of erosion relative to hydrophobic polymers. Hydrophobic polymers are believed to have almost purely surface diffusion of active agent, having erosion from the surface inwards. Hydrophilic polymers are believed to allow water to penetrate the surface of the polymer, allowing hydrolysis of labile bonds beneath the surface, which can lead to homogeneous or bulk erosion of polymer.

The implantable device coating can include a blend of polymers each having a different release rate of the therapeutic agent. For instance, the coating can include a polylactic acid/polyethylene oxide (PLA-PEO) copolymer and a polylactic acid/polycaprolactone (PLA-PCL) copolymer. The polylactic acid/polyethylene oxide (PLA-PEO) copolymer can exhibit a higher release rate of therapeutic agent relative to the polylactic acid/polycaprolactone (PLA-PCL) copolymer. The relative amounts and dosage rates of therapeutic agent delivered over time can be controlled by controlling the relative amounts of the faster releasing polymers relative to the slower releasing polymers. For higher initial release rates the proportion of faster releasing polymer can be increased relative to the slower releasing polymer. If most of the dosage is desired to be released over a long time period, most of the polymer can be the slower releasing polymer. The stent can be coated by spraying the stent with a solution or dispersion of polymer, active agent, and solvent. The solvent can be evaporated, leaving a coating of polymer and active agent. The active agent can be dissolved and/or dispersed in the polymer. In some embodiments, the co-polymers can be extruded over the stent body.

Optionally, compounds of formula (I) can be administered in conjunction with one or more other agents that inhibit the TGFβ signaling pathway or treat the corresponding pathological disorders (e.g., fibrosis or progressive cancers) by way of a different mechanism of action. Examples of these agents include angiotensin converting enzyme inhibitors, nonsteroid and steroid anti-inflammatory agents, as well as agents that antagonize ligand binding or activation of the TGFβ receptors, e.g., anti-TGFβ, anti-TGFβ receptor antibodies, or antagonists of the TGFβ type II receptors.

The invention will be further described in the following examples, which do not limit the scope of the invention described in the claims. The full contents of all publications cited herein, including but not limited to scientific articles, issued patents or published patent applications, are incorporated herein by reference in their entirety.

### EXAMPLES

### Preparation 1

### 1-Allyl-4-(tert-butyl-dimethyl-silanyloxy)-cyclohexanecarbaldehyde

### Step a: 4-(tert-Butyl-dimethyl-silanyloxy)-cyclohexanecarboxylic acid ethyl ester

To a 50 mL flask was added 4-hydroxy-cyclohexanecarboxylic acid ethyl ester (5.0 g; 29.0 mmol), imidazole (2.37 g; 34.8 mmol), DMF (30 mL) and tert-butyldimethylsilyl chloride (4.81 g; 32.0 mmol). The solution was stirred for 78 h at 20° C. The reaction was diluted with EtOAc (100 mL), washed with water 2×100 mL), brine (100 mL), dried (MgSO₄) filtered and concentrated in vacuo. The crude oil was purified by flash chromatography (silica gel, hexanes/EtOAc 1:0 to 1:1) to give pure product (7.79 g, 94% yield) as a 2:1 mixture of diastereomers.

¹H-NMR (400 MHz, CDCl₃) (J=Hz) δ mixture 4.12 (m; 2H), 3.90 (m; 0.65H), 3.58 (m; 0.35H), 2.23 (m; 1H), 1.98 (m; 3H), 1.82 (m; 3H), 1.49 (m; 2H), 1.21 (m; 3H), 0.88 (s; 9H), 0.05 (s; 3H), 0.03 (s; 31-1). ¹³C-NMR (400 MHz, CDCl₃) δ mixture 175.7, 175.6, 70.5, 66.6, 60.1, 60.0, 42.2, 42.1, 34.8, 32.8, 27.2, 25.8, 25.6, 23.4, 18.2, 18.0, 14.2, −4.7, −4.9. MS (ES+) m/z 287.22 [MH+].

### Step b: 1-Allyl-4-(tert-butyldimethylsilanyloxy)-cyclohexanecarboxylic acid ethyl ester

To a 100 mL flask was added ester (4.53 g; 15. 8 mmol) and THF (25 mL). The mixture was cooled to −78° C. and then a 1.0 M solution of LiHMDS (16.6 mL; 16.6 mmol) in THF was added. The reaction was stirred for 1 hour at −78° C. and then allyl bromide (1.52 mL; 17.4 mmol) was added. The reaction mixture was stirred over 4 hrs while warming to 20° C. The reaction was diluted with ether (100 mL) and washed with 5% HCl (100 mL), brine (100 mL), dried (MgSO₄), filtered and concentrated in vacuo. The crude oil was purified by flash chromatography (silica gel, hexanes/EtOAc 1:0 to 1:1) to give pure product (2.57 g, 50% yield) as a 10:1 mixture of diastereomers.

¹H-NMR (400 MHz, CDCl₃) (J=Hz) δ major 5.70 (m; 1H), 5.02 (m; 2H), 4.15 (m; 2H), 3.56 (m; 1H), 2.20 (d; 2H), 1.95 (m; 2H), 1.77 (m; 2H), 1.52 (m; 2H), 1.35 (m; 2H), 1.23 (m; 3H), 1.20 (m; 1H), 0.91 (s: 9H), 0.02 (s; 6H). ¹³C-NMR (400 MHz, CDCl₃) δ major 175.4, 133.7, 117.6, 70.8, 60.2, 46.4, 32.8, 31.7, 30.9, 25.8, 18.2, 14.3, −4.7, −4.9. MS (ES+) 324.02 m/z [MH+].

### Step c: [1-ally-4-(tert-butyl-dimethyl-silanyloxy)-cyclohexyl]-methanol

To a 100 mL flask was added ethyl ester (2.57 g; 7.87 mmol) and toluene (25 mL). The solution was cooled to −78° C. and a 1.0 M solution of DIBAL-H in toluene (23.6 mL; 23.6 mmol) was added. The solution was stirred for 30 m at −78° C. and then warmed to room temperature. The reaction was diluted with EtOAc (100 mL) and solid Na₂SO₄ was added, followed by 10 ml of water. The mixture was stirred to a slurry and then filtered through a silica gel pad and concentrated in vacuo. The crude oil was purified by flash chromatography (silica gel, hexanes/EtOAc 1:0 to 1:1) to give pure product (1.98 g, 88% yield) as a single diastereomer.

¹H-NMR (400 MHz, CDCl₃) (J=Hz) δ 5.83 (m; 1H), 5.05 (m; 2H), 3.64 (m; 1H), 3.50 (s; 2H), 2.08 (d; 2H; J=7.5), 1.62 (m; 4H), 1.43 (m; 2H), 1.20 (m; 2H), 0.91 (s; 9H), 0.05 (s; 6H). ¹³C-NMR (400 MHz, CDCl₃) δ. 135.3, 117.2, 70.0, 66.7, 41.3, 37.1, 30.5, 29.1, 25.9, 18.2, −4.6. MS (ES+) 285.33 m/z [MH+].

### Step d: 1-Allyl-4-(tert-butyl-dimethyl-silanyloxy)-cyclohexanecarbaldehyde

To a 200 mL flask was added alcohol (1.98 g; 7.0 mmol), Dess-Martin periodinane (2.95 g; 7.0 mmol), and methylene chloride (100 mL). The solution was stirred for 5 hrs at 20° C. The reaction was diluted with ether (500 mL), filtered through celite and concentrated in vacuo. The crude oil was purified by flash chromatography (silica gel, hexanes/EtOAc 1:0 to 10:1) to give pure product (1.17 mg, 59% yield) as a single diastereomer.

¹H-NMR (400 MHz, CDCl₃) (J=Hz) δ 9.46 (s; 1H), 5.66 (m; 1H), 5.05 (m; 2H), 3.58 (m; 1H), 2.15 (d; 2H; J=6.8) δ. 2.04 (m; 2H), 1.73 (in; 2H), 1.30 (m; 4H), 0.90 (s; 9H), 0.04 (s; 6H). ¹³C-NMR (400 MHz, CDCl₃) δ. 206.1, 132.5, 118.5, 70.0, 48.9, 40.6, 31.8, 30.2, 28.3, 25.8, 18.1, −4.8.

### Preparation 2

### 1-(2-(Benzyloxy)ethyl)-4-(t-butyldimethylsilyloxy)cyclohexanecarbaldehyde

### Step a: Ethyl 4-(tert-butyldimethylsilyloxy)cyclohexanecarboxylate

A mixture of ethyl 4-hydroxycyclohexane-carboxylate (50 mL, 0.31 mol), imidazole (50.1 g, 0.74 mol), and t-butyldimethylsilyl chloride (56 g, 0.37 mol) in DMF (580 mL) was stirred at room temperature for 20 hrs under atmosphere of nitrogen. Water (100 mL) was added to the mixture, and the mixture was extracted with ether (600 mL). The extract was washed with water (400 mL) and brine (500 mL), dried over anhydrous Na₂SO₄, and filtered. The filtrate was concentrated to give compound 2 as a colorless oil (102 g), which was used for the next step without further purification.

### Step b: Ethyl 1-(2-(benzyloxy)ethyl)-4-(tert-butyldimethylsilyloxy)cyclohexanecarboxylate

To a mixture of the compound of Step a (102 g, 0.357 mol) in THF (800 mL) at −78° C. was added lithium bis(trimethylsiyl)amide (438 mL) dropwise under nitrogen. The mixture was stirred for 30 min followed by the addition of benzyl 2-bromoethyl ether (109 mL, 0.375 mol). After stirring the mixture for another 30 min at −78° C., the reaction mixture was allowed to warm up to room temperature and stirred for 1 h. The mixture was diluted with ethyl acetate (1,000 mL) and washed with brine (1,000 mL), dried over Na₂SO₄ and filtered. The filtrate was concentrated to give a crude product which was purified by flash chromatography (silica gel, EtOAc/Hexane:0/1 to 1/1) to give the ester-benzylether as a colorless oil (115 g, 73%).

### Step c: (1-(2-(Benzyloxy)ethyl)-4-(tert-butyldimethylsilyloxy)cyclohexyl)methanol

To a solution of the compound of Step b (115 g, 0.273 mol) in anhydrous toluene (1,700 mL) at −78° C. was added Diisobutylaluminum hydride (900 mL, 1M, in toluene) under an atmosphere of nitrogen. The mixture was stirred for 45 min, and the reaction was quenched by adding ethyl acetate. The mixture was poured into saturated aqueous Na₂SO₄ (2,000 mL). The mixture was allowed to stand for 1 hr, and filtered through a silica gel pad. The filtrate was extracted with ethyl acetate. The organic layers were separated, washed with brine, dried over anhydrous Na₂SO₄ and filtered. The solvent of the filtrate was removed on vacuum to give alcohol as a colorless oil (57.13 g, 55.8%).

### Step d: 1-(2-(Benzyloxy)ethyl)-4-(tert-butyldimethylsilyloxy)cyclohexanecarbaldehyde

To a solution of the compound of Step c (45 g, 0.12 mol) in methylene chloride (800 mL) was added Dess-Martin periodinane (66 g, 0.16 mol) slowly. The reaction mixture was stirred at room temperature for 1 hr, and then ether (500 mL) was added. The mixture was stirred for 10 min. After removal of the majority of the solvents by rotary evaporation, ether (200 mL) was added. The mixture was filtered, and the solid was washed with ether. The filtrate was washed with aqueous sodium thiosulfate, brine, dried over anhydrous sodium sulfate and concentrated under reduced pressure to give a crude product which was purified via flash chromatography (silica gel, EtOAc/Hexane:0/1 to 1/1) to afford the title aldehyde as a colorless oil (22 g: 48.8%). ¹H-NMR (300 MHz, CDCl₃): 9.48 (s, 1H), 7.30 (m, 5H), 4.41 (s, 2H), 3.58 (m, 1H), 3.42 (t, 2H, J=6.6), 2.07 (m, 2H), 1.74 (m, 2H), 1.30 (m, 2H), 0.87 (s, 9H), 0.037 (s, 6H).

### Preparation 3

### 4-Formyl-4-(2-methoxyethyl)-1-(tert-butoxycarbonyl)piperidine

### Step a; 4-(2-Methoxy-ethyl)-piperidine-1,4-dicarboxylic acid 1-tert-butyl ester 4-methyl ester

To a 1-neck round-bottom flask was added Piperidine-1,4-dicarboxylic acid 1-tert-butyl ester 4-methyl ester (3.0 g, 0.012 mol) in tetrahydrofuran (60 mL). The reaction was cooled at −78° C. and lithium diisopropylamide in tetrahydrofuran (0.5 M, 27 mL) added. The reaction was stirred at −78° C. for 30 minutes and 1-bromo-2-methoxyethane (1.4 mL, 0.015 mol) was added. The solution was stirred at −78° C. for 1 hour, warmed to room temperature and kept for 5 hrs. The solvent was concentrated and the residue extracted with aqueous NH₄HCO₃/CH₂Cl₂. The organic layer was dried by Na₂SO₄ and filtered. The solution was concentrated to dryness to give a crude product.

### Step b: 4-Formyl-4-(2-methoxyethyl)-piperidine-1-carboxylic acid tert-butyl ester

Into a 1-neck round-bottom flask was added 4-(2-methoxyethyl)-piperidine-1,4-dicarboxylic acid, 1-tert-butyl ester, and 4-methyl ester (3.4 g, 0.011 mol) in methylene chloride (50 mL, 0.8 mol). The solution was cooled at −78° C. and 1 M of diisobutylaluminum hydride in methylene chloride (36 mL) was added. The reaction was stirred at −78° C. for 0.5 h and 8 mL isopropanol was added. The reaction was then warmed up to room temperature for 1 h then 10 mL 1N HCl was added. A standard methylene chloride extraction was performed. The organic layer was dried with Na₂SO₄ and filtered. The solvent was concentrated and the residue dried under vacuum overnight to give 3.0 g crude product.

¹H-NMR (300 MHz, CDCl₃) (J=Hz) δ 9.40 (s; 1H), 3.70 (m; 2H), 3.36 (m; 2H), 3.18 (s; 3H), 2.95 (m; 2H), 1.90 (m; 2H), 1.74 (m; 2H), 1.59 (m; 2H), 1.41 (s; 9H).

### Example 1

### (1s,4s,Z)-3′-(Benzo[d][1,3]dioxol-5-yl)-2′(6-methylpyridin-2-yl)-5′,8′-dihydrospiro[cyclohexane-1,9′-iimidazo[1,2-a]azepin]-4-ol

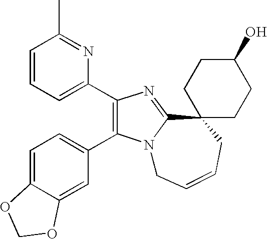

### Step 1a: 1-Allyl-2-[1-allyl-4-(tert-butyl-dimethyl-silanyloxy)-cyclohexyl]-4-(6-methyl-pyridin-2-yl)-5-benzo[1,3]dioxol-5-yl-1H-imidazole

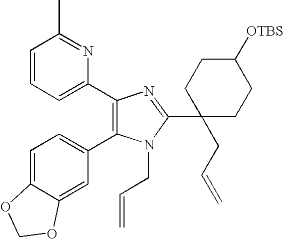

A 250-mL round-bottom flask was charged with 1-benzo[1,3]dioxol-5yl-2-(6-methyl-pyridin-2yl)-ethane-1,2-dione (1.0 g, 4.0 mmol), 1-allyl-4-(tert-butyl-dimethyl-silanyloxy)-cyclohexanecarbaldehyde (1.1 g, 4.0 mmol), allylamine (0.27 g, 4.8 mmol), MTBE (55 mL), AcOH (4 mL) and ammonium acetate (0.34 g, 4.4 mmol). The reaction was heated to 65° C. for 12 hrs and then cooled to room temperature. The reaction mixture was extracted with EtOAc (150 mL) and the organic was washed with brine (100 mL), saturated NaCl (100 mL), dried (MgSO₄), filtered and concentrated in vacuo. The crude product was purified by flash chromatography silica gel, hexanes/EtOAc 1:0 to 0:1) to give 1.16 g (51%) of pure product.

### Step 1b: 9-[4-(tert-butyl-dimethyl-silanyloxy)-cyclohexyl]-3-benzo[1,3]dioxol-5-yl-2-(6-methylpyridin-2-yl)-8,9-dihydro-5H-imidazo[1,2-z]azepine

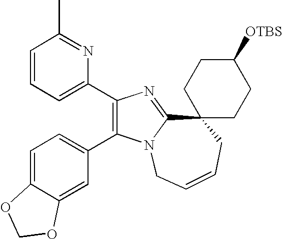

A 100-mL round-bottom flask was charged with 1-allyl-2-[1-allyl-4-(tert-butyl-dimethyl-silanyloxy)-cyclohexyl]-4-(6-methyl-pyridin-2-yl)-5-benzo[1,3]dioxol-5-yl-1H-imidazole (0.3 g, 0.5 mmol), Grubb's 1^{st} catalyst (0.3 g), and methylene chloride (20 mL). The reaction mixture was stirred at room temperature for 48 hrs. The mixture was filtered through silica gel pad and eluted with EtOAc (50 mL). The crude product was purified by flash chromatography (silica gel, hexanes/EtOAc 1:0 to 0:1) to give 0.18 g (60%) of the title compound.

### Step 1c: (1s,4s,Z)-3′-(Benzo[d][1,3]dioxol-5-yl)-2′-(6-methylpyridin-2-yl)-5′,8′-dihydrospiro[cyclohexane-1,9′-imidazo[1,2-a]azepin]-4-ol

The silyl compound of Step 1b (60 mg) was dissolved in 0.1% trifluoroacetic acid in 1:1 acetonitrile:water. After 1 hour, the mixture was lypholized to provide the title compound as the trifluoroacetate salt.

¹H-NMR (400 MHz, CDCl₃), (J=Hz), δ 7.91 (t, 1H, J=8.0), 7.37 (d; 1H, J=8.0), 7.17 (d; 1H, J=8.4), 7.00 (d, 1H, J=7.6), 6.84 (d; 1H; J=8.4), 6.80 (s; 1H), 6.15 (s; 2H), 5.98 (m; 1H), 5.74 (m; 1H), 4.48 (d, 2H, J=5.2), 3.96 (s, 1H), 2.86 (d, 2H, J=5.2), 2.83 (s; 3H), 1.96 (m, 4H), 1.69 (m, 4H), 1.53 (s; 1H). MS (ES+) m/z 430.21 [MH+].

### Example 2

### (1s,4r)-3′-(Benzo[d][1,3]dioxol-5-yl)-2′-(6-methylpyridin-2-yl)-5′,6′,7′,8′-tetrahydrospiro[cyclohexane-1,9′-imidazo[1,2-a]azepin]-4-ol

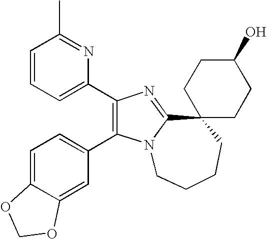

### Step 2a: 3′-(Benzo[d][1,3]dioxol-5-yl)-4-(tert-butyldimethylsilyloxy)-2′-(6-methylpyridin-2-yl)-5′,6′,7′,8′-tetrahydrospiro[cyclohexane-1,9′-imidazo[1,2-a]azepine]

A 100-mL pressure flask was charged with 9-[4-(tert-butyl-dimethyl-silanyloxy)-cyclohexyl]-3-benzo[1,3]dioxol-5-yl-2-(6-methylpyridin-2-yl)-8,9-dihydro-5H-imidazo[1,2-z]azepine (120 mg, 0.22 mmol), palladium on charcoal (10%) (0.2 g), methanol (15 mL). The reaction mixture was stirred under atmosphere of hydrogen (60 psi) for 2 h. The catalyst was filtered out. The crude product was purified by flash chromatography silica gel, hexanes/EtOAc 1:0 to 0:1) to give (94 mg, 78%) pure product.

### Step 2b

The silanyl protecting group was removed according to the procedure described in Step 1c to provide the title compound.

¹H-NMR (400 MHz, CDCl₃), (J=Hz), δ 7.80 (t, 1H, J=6.8), 7.22 (d; 1H, J=7.2), 7.05 (d; 1H, J=8.0), 6.95 (d, 1H, J=7.6), 6.87 (d; 1H; J=7.2), 6.77 (s; 1H), 6.12 (s; 2H), 3.92 (m, 2H), 3.86 (s, 1H), 2.80 (s; 3H), 2.53 (m; 2H), 1.88 (m, 4H), 1.76 (m, 2H), 1.50 (s, 1H), 1.44 (t, 2H, J=5.8), 1.27 (m, 4H). MS (ES+) m/z 432.22 [MH+].

### Example 3

### (1s,4s,Z)-2′-(Benzo[d][1,3]dioxol-5-yl)-3′-(6-methylpyridin-2-yl)-5′,8′-dihydrospiro[cyclohexane-1,9′-imidazo[1,2-a]azepin]-4-ol

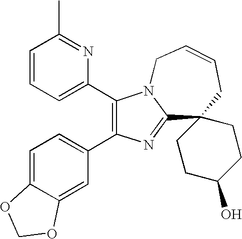

### Step 3a: 2-{(2-[1-Allyl-4-(tert-butyl-dimethyl-silanyloxy)-cyclohexyl]-5-benzo[1,3]dioxol-5-yl-1H-imidazol-4-yl}-6-methyl-pyridine

To a 100 mL flask was added aldehdye (829 mg; 2.44 mmol), MTBE (24 mL), diketone (658 mg; 2.93 mmol), ammonium acetate (1.88 g; 24.4 mmol) and AcOH (6 mL). The solution was stirred for 24 hrs at reflux. The reaction mixture was diluted with EtOAc (50 mL), washed with water (50 mL), brine (50 mL), dried (MgSO₄), filtered and concentrated in vacuo. The crude oil was purified by flash chromatography (silica gel, hexanes/EtOAc 1:0 to 1:1) to give pure product (507 mg, 39% yield) as a single diastereomer.

¹H-NMR (400 MHz, CDCl₃) (J=Hz) δ 7.46 (t; 1H; J=7.5), 7.21 (d; 1H; J=8.0), 7.06 (m; 2H), 6.98 (d; 1H; J=7.3), 6.83 (d; 1H; J=7.4), 5.99 (s; 2H), 5.62 (m; 1H), 5.00 (m; 2H), 3.68 (m; 1H), 2.54 (s; 2H), 2.48 (m; 2H), 2.33 (m; 2H), 2.07 (s; 3H), 1.80 (m; 2H), 1.53 (m; 4H), 0.87 (s; 9H), 0.05 (s; 6H). ¹³C-NMR (400 MHz, CDCl₃) δ 157.5, 152.7, 148.5, 147.6, 147.3, 139.6, 137.5, 133.9, 128.3, 124.6, 123.0, 121.5, 119.1, 117.9, 109.8, 108.4, 101.0, 60.4, 40.0, 32.5, 32.2, 25.9, 23.4, 21.1, 18.3, −4.8. MS (ES+) m/z 532.16 [MH+].

### Step 3b: 2-(1-Allyl-2-(1-allyl-4-(tert-butyldimethylsilyloxy)cyclohexyl)-4-(benzo[d][1,3]dioxol-5-yl)-1H-imidazol-5-yl)-6-methylpyridine

A 100-mL round-bottom flask was charged with 2-[1-allyl-4-(tert-butyl-dimethyl-silanyloxy)cyclohexyl]-4-(6-methylpyridin-2-yl)-5-benzo[1,3]dioxol-5-yl-1H-imidazole (0.40 g, 0.75 mmol), allyl bromide (0.20 g, 1.7 mmol), sodium hydride (38 mg, 1.5 mmol), and DMF (30 mL). The reaction was heated to 65° C. for 12 hrs under atmosphere of nitrogen and then cooled to room temperature. The reaction mixture was quenched with water (20 mL) and extracted with EtOAc (70 mL) and the organic was washed with sat. NaCl (50 mL), dried (MgSO₄), filtered and concentrated in vacuo. The crude product was purified by flash chromatography (silica gel, hexanes/EtOAc 1:0 to 0:1) to give 0.29 g (68%) of purified product.

### Step 3c: (1s,4s,Z)-2′-(Benzo[d][1,3]dioxol-5-yl)-4-(tert-butyldimethylsilyloxy)-3′-(6-methylpyridin-2-yl)-5′,8′-dihydrospiro[cyclohexane-1,9′-imidazo[1,2-a]azepine]

A ring closure metathesis was conducted to product of Step 3b according to the procedure of Step 1b, to provide this intermediate.

### Step 3d: (1s,4s,Z)-2′-(Benzo[d][1,3]dioxol-5-yl)-3′-(6-methylpyridin-2-yl)-5′,8′-dihydrospiro[cyclohexane-1,9′-imidazo[1,2-a]azepin]-4-ol

The silyl protecting group was removed according to the procedure of Step 1c to provide the title compound.

¹H-NMR (400 MHz, CDCl₃) (J=Hz), δ 7.66 (t, 1H, J=7.8), 7.30 (d; 1H, J=7.2), 7.11 (d; 1H, J=7.6), 6.92 (d, 1H; J=8.4), 6.77 (d; 1H; J=8.4), 6.73 (s; 1H), 6.22 (m; 1H), 6.04 (m; 1H), 5.97 (s; 2H), 4.85 (d, 2H, J=5.2), 4.02 (s, 1H), 2.74 (d, 21-1, J=5.2), 2.70 (s; 3H), 1.95 (m, 4H), 1.53 (s; 1H), 1.45 (m, 4H). MS (ES+) m/z 430.21 [MH+].

### Example 4

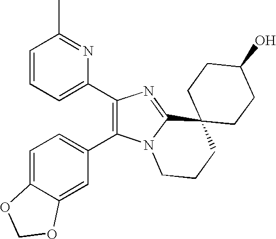

### Step 4a: 3-(1-(4-(Benzo[d][1,3]dioxol-5-yl)-5-(6-methylpyridin-2-yl)-1H-imidazol-2-yl)-4-(tert-butyldimethylsilyloxy)cyclohexyl)propan-1-ol

A 100-mL flask was charged with 2-{2-[1-allyl-4-(tert-butyl-dimethyl-Silanyloxy)-cyclohexyl]-4-(6-methyl-pyridin-2-yl)-5-{1-[4-benzo[1,3]dioxol-5-yl-3H-imidazole (0.30 g, 0.56 mmol), and THF (30 mL) under atmosphere of nitrogen. 9-BBN dimer (0.73 g, 3.0 mmol) was then added. The mixture was stirred at room temperature for 30 minutes. Hydrogen peroxide (2 mL, 30%) and sodium hydroxide (2 mL, 6 N) were introduced. The mixture was heated at 65° C. for 1 hour. The mixture was cooled down and diluted with NaHCO₃ and extracted with ethyl acetate (75 mL). The crude product was purified by flash chromatography (silica gel, hexanes/EtOAc 1:0 to 0:1) to give 0.23 g (75%) pure product.

### Step 41),

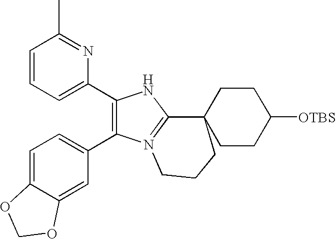

A 250-mL flask was charged with triphenylphosphine (0.52, 2.0 mmol) imidazole (0.14 g, 2.0 mmol) and methylene chloride (50 mL). After the mixture was stirred for 5 minutes, iodine (0.51 g, 2.0 mmol) was added. The mixture was stirred at room temperature for 10 minutes. The product of Step 4a (0.50 g, 0.91 mmol) in methylene chloride (10 mL) was introduced. The mixture was allowed to stir at room temperature for 10 minutes. The mixture was washed with dilute Na₂S₂O₃ and brine. The crude product was purified by flash chromatography (silica gel, hexanes/EtOAc 1:0 to 0:1) to give 0.42 (85%) the title compound.

### Step 4c

The silyl protecting group of the product of Step 4b was removed according to the procedure of Step 1c to provide the tile compound.

¹H-NMR (400 MHz, DMSO-d6), (J=Hz), δ 7.78 (t, 1H, J=7.8), 7.33 (d, 1H, J=8.4), 7.17 (d, 1H, J=7.6), 6.95 (d, 1H, J=8.0), 6.89 (d, 1H, J=7.6), 6.85 (s, 1H), 6.09 (s, 2H), 4.14 (m, 1H), 3.83 (t, 2H, J=7.8), 2.69 (s, 3H), 2.57 (t, 2H, J=11.2), 2.06 (m, 2H), 1.84 (m, 2H), 1.69 (m, 2H). MS (ES+) m/z 418.21 [MH+].

### Example 5

### (1r,4s)-3′-(benzo[d][1,3]dioxol-5-yl)-2′-(6-methylpyridin-2-yl)-6′,7′-dihydro-5′H-spiro[cyclohexane-1,8′-imidazo[1,2-a]pyridin]-4-ol

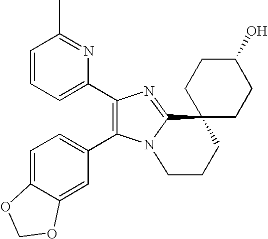

A 25-mL flask was charged with the product of Step 4c (30 mg, 0.072 mmol) in THF (5 mL), 4-nitrobenzoic acid (0.2 g, 1.2 mmol), triphenylphosphine (0.10 g, 0.62 mmol) and diethyl azodicarboxylate (0.10 g, 0.57 mmol). The mixture was allowed to stir at room temperature for 4 hrs. The mixture was then diluted with EtOAc and washed with brine. The crude product was purified by chromatography (silica gel, 1:0 to 0:1, hexane/EtOAc) to yield 34.6 mg (85%). The purified product was dissovled in acetonitrile (5 mL) with Na₂CO₃ (0.3 g) and stirred for 2 hrs at room temperature. The mixture was diluted with EtOAc and washed with brine. The crude product was purified with prep. HPLC to yield 18 mg (70%) of the title compound.

¹H-NMR (400 MHz, DMSO-d6), (J=Hz), δ 7.70 (t, 1H, J=7.6), 7.30 (d, 111, J=8.0), 7.14 (s, 1H), 7.11 (d, 1H, J=8.0), 6.99 (d, 1H, J=7.6), 6.97 (t, 1H, J=7.6), 6.15 (s, 2H), 4.04 (m, 1H), 3.77 (s, 2H), 3.62 (m, 1H), 2.58 (s, 3H), 2.26 (t, 2H, J=12.0), 1.81 (d, 2H, J=1.1.6)), 1.44 (m, 2H), 1.59 (t, 2H, J=13.6). MS (ES+) m/z 418.21 [MH+].

### Example 6

### 3′-(Benzo[d][1,3]dioxol-5-yl)-2′-(6-methylpyridin-2-yl)-6′,7′-dihydro-5′H-spiro[cyclohexane-1,8′-imidazo[1,2-a]pyridin]-4-one

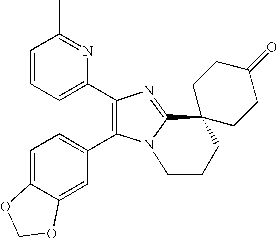

A mixture of the product of Step 4c (0.21 g, 0.50 mmol) and Dess-Martin periodinane (0.32 g, 0.75 mmol) in methylene chloride (15 mL) was stirred at room temperature for 1 h. TLC and MS showed about 60% conversion. Additional Dess-Martin periodinane (0.22 g) was added and the mixture stirred for another 0.5 hour. LC-MS showed only oxidized product. Aqueous Na₂SO₃ was added, stirred for 20 mins. The solvent was concentrated and the residue dissolved in ethyl acetate (20 ml) and was washed with aqueous Na₂CO₃. The organic layer was dried over Na₂SO₄ and filtered. The filtrate was concentrated to dryness and the residue chromatographed (Acetone/Hexanes 1:1) to give 0.1 g of the title compound (HPLC-85%).

¹H-NMR (300 MHz, CDCl₃) (J=Hz) δ 7.92 (t, 1H, J=8.1), 7.36 (d, 1H, J=7.8), 7.24 (d, 1H, J=7.8), 6.97 (d, 1H, J=7.8), 6.86 (d, 1H, J=7.6), 6.85 (s, 1H), 6.10 (s, 2H), 3.84 (br, 2H), 2.80 (m, 5H), 2.50 (m, 4H), 2.15 (m, 6H).

MS (ES+) m/z 416.0 [MH+].

### Example 7

### 11H-3′-(Benzo[d][1,3]dioxol-5-yl)-2′-(6-methylpyridin-2-yl)-6′,7′-dihydro-5′H-spiro[cyclohexane-1,8′-imidazo[1,2-a]pyridin]-4-one oxime

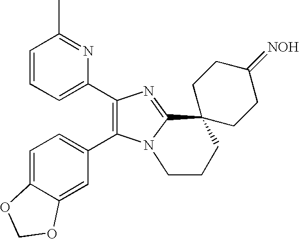

The ketone of Example 6 (50 mg) was dissolved in ethanol (3 mL) and hydroxylamine hydrochloride (0.01 g) added. The mixture was held at room temperature for 1 hour. Aqueous Na₂SO₃ solution was added and the mixture stirred for 5 mins. The mixture was extracted with methylene chloride, the organic layer was dried over Na₂SO₄ and concentrated to dryness to give 22 mg of the title compound (HPLC-85%).

MS (ES+) m/z 431.0 [MH+].

### Example 8

### (S)-3′-(Benzo[d][1,3]dioxol-5-yl)-2′-(6-methylpyridin-2-yl)-6′,7′-dihydro-5′H-spiro[azepane-4,8′-imidazo[1,2-a]pyridin]-7-one

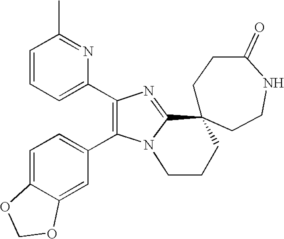

The oxime of Example 7 (22 mg) was dissolved in acetone (0.7 mL, 0.01 mol). p-Toluenesulfonyl chloride (22 mg, 0.12 mmol) in 0.2 mL acetone and sodium bicarbonate in water (0.33 M, 0.7 mL) were added at 0° C. over 2 mins. The reaction was stirred at RT overnight. Na₂SO₃ aq. was added, stirred for 5 mins. The mixture was extracted with CH₂Cl₂. The organic layer was dried by Na₂SO₄ and was filtered. The solution was concentrated to dryness and the residue (18 mg) was purified by HPLC to give 9.0 mg of the title compound.

¹H-NMR (300 MHz, MeOD) (J=Hz) δ 7.69 (t, 1H, J=7.9), 7.26 (d, 1H, J=7.8), 7.02 (d, 1H, J=8.1), 6.94 (d, 1H, J=8.0), 6.87 (s, 1H), 6.85 (d, 1H, J=7.8), 5.95 (s, 2H), 3.74 (t, 2H, J=5.7), 3.43 (m, 1H), 3.30 (m, 1H), 2.70 (m, 1H), 2.59 (s, 3H), 2.45 (m, 1H), 2.40-1.85 (m, 8H). MS (ES+) m/z 431.04 [MH+].

### Example 9

### 3′-(benzo[d][1,3]dioxol-5-yl)-2′-(6-methylpyridin-2-yl)-5′,6′-dihydrospiro[cyclohexane-1,7′-pyrrolo[1,2-a]imidazol]-4-ol

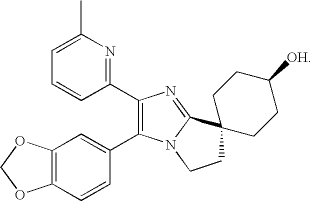

### Step 9a: 2-(5-(Benzo[d][1,3]dioxol-5-yl)-2-(1-(2-(benzyloxy)ethyl)-4-(tert-butyldimethylsilyloxy)cyclohexyl)-1H-imidazol-4-yl)-6-methylpyridine

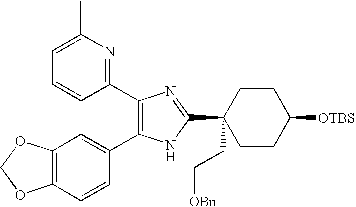

The title compound was made following the procedure of Step 3a. MS (ES+) m/z 626.50 [MH+].

### Step 9b: 2-(1-(5-(benzo[d][1,3]dioxol-5-yl)-4-(6-methylpyridin-2-yl)-1H-imidazol-2-yl)-4-(tert-butyldimethylsilyloxy)cyclohexyl)ethanol

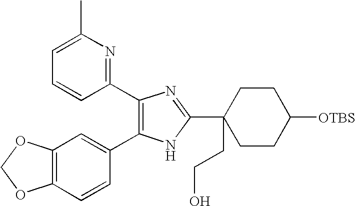

A 100-mL pressure flask was charged with the imidazole of Step 9a (0.30 g, 0.48 mmol), palladium on charcoal (10%) (0.2 g), and acetic acid (15 mL). The mixture was stirred under atmosphere of hydrogen (60 psi) for 12 h. The catalyst was filtered, the filtrate concentrated and the crude product purified by flash chromatography on silica gel (hexanes/EtOAc 1:0 to 0:1) to give 0.19 g (75%) pure product. MS (ES+) m/z 536.4 [MH+].

### Step 9c: 3′-(Benzo[d][1,3]dioxol-5-yl)-2′-(6-methylpyridin-2-yl)-5′,6′-dihydrospiro[cyclohexane-1,7′-pyrrolo[1,2-a]imidazol]-4-ol

The title compound was prepared from the product of Step 9b by the cyclization procedure of Step 4b, followed by deprotection according to the procedure of Step 1c. ¹H-NMR (400 MHz, CDCl₃), (J=Hz), δ 7.61 (t, 1H, J=7.86), 7.53 (d; 1H, J=7.6), 7.21 (s; 1H), 7.03 (t, 1H, J=7.6), 7.01 (s; 1H), 6.93 (d, 1H, J=8.0), 6.05 (s, 2H), 4.55 (s, 1H), 3.96 (t, 2H, J=6.2), 3.64 (s, 1H), 2.51 (s, 3H), 2.35 (t, 2H, J=6.0), 1.95 (m, 4H), 1.65 (m, 2H), 1.49 (t, 2H, J=10.0).

MS (ES+) m/z 404.19 [MH+].

### Example 10

### 6-((1s,4s)-2′-(5-Fluoro-6-methylpyridin-2-yl)-4-hydroxy-5′,6′-dihydrospiro[cyclohexane-1,7′-pyrrolo[1,2-a]imidazole]-3′-yl)-3-methylquinazolin-4(3H)-one

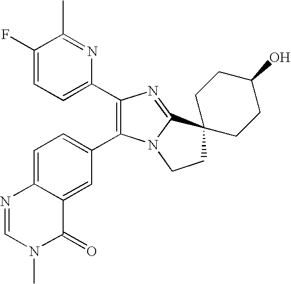

The title compound was prepared as described in Example 9.

1H-NMR (400 MHz, DMSO-d6), (J=Hz), δ 8.44 (s, 1H), 8.28 (s, 1H), 7.90 (d, 1H, J=8.4), 7.85 (d, 1H, J=8.0), 7.36 (t, 1H, J=8.8), 7.24 (d-d, 1H, J=8.8, 3.6), 4.17 (t, 2H, J=7.0), 4.00 (s, 1H), 3.55 (s, 3H), 2.70 (t, 2H, J=7.2), 2.49 (s, 3H), 2.45 (m, 2H), 1.97 (m, 2H), 1.84 (m, 2H), 1.74 (m, 2H). MS (ES+) m/z 460.21 [MH+].

### Example 11

### (1s,4s)-2′-(5-Fluoro-6-methylpyridin-2-yl)-3′-(3-methyl-4-oxo-3,4-dihydroquinazolin-6-yl)-5′,6′-dihydrospiro[cyclohexane-1,7′-pyrrolo[1,2-a]imidazole]-4-yl acetate

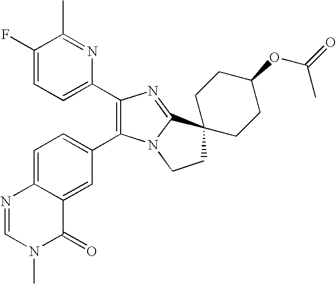

The compound from Example 10, acetic anhydride (1.5 equiv.) and triethylamine (2.0 equiv.) in methylene chloride was kept overnight. The mixture was washed with water, dried and concentrated to give the title compound.

¹H-NMR (400 MHz, CDCl₃), (J=Hz), δ 8.46 (d, 2H, J=11.8), 7.88 (s, 2H), 7.34 (t, 1H, J=5.6), 7.28 (d, 1H, J=7.6), 4.26 (t, 2H, J=6.4), 3.70 (s, 3H), 3.51 (s, 1H), 2.76 (t, 2H, 6.6), 2.52 (t, 2H, J=6.8), 2.42 (s, 3H), 2.35 (m, 2H), 2.09 (s, 3H), 2.02 (s, 3H), 1.74 (t, 2H, 10.2), 1.56 (t, 2H, J=6.8). MS (ES+) m/z 484.23 [MH+].

### Example 12

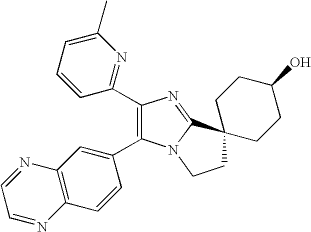

The title compound was prepared as described in Example 9.

¹H-NMR (400 MHz, DMSO-d6), (J=Hz), δ 8.99 (d-d, 2H, J=6.4, 2.4), 8.33 (d, 1H, J=2.4), 8.28 (d, 1H, J=8.8), 7.93 (d-d, 1H, J=8.8, 2.0), 7.82 (t, 1H, J=8.2), 7.39 (d, 1H, J=8.0), 7.33 (d, 1H, J=8.4), 4.13 (t, 2H, J=7.6), 3.90 (m, 1H), 2.73 (s, 3H), 2.61 (t, 2H, J=7.2), 2.27 (m, 2H), 2.03 (m, 2H), 1.83 (m, 2H), 1.80 (m, 2H). MS (ES+) m/z 412.21 [MH+].

### Example 13

### (1r,4r)-2′-(6-methylpyridin-2-yl)-3′-(quinoxalin-6-yl)-5′,6′-dihydrospiro[cyclohexane-1,7′-pyrrolo[1,2-a]imidazol]-4-amine

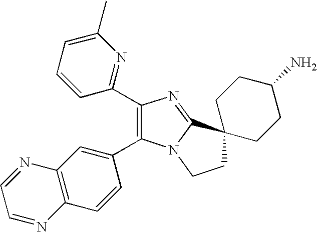

### Step 13a: (1s,4s)-2′-(6-methylpyridin-2-yl)-3′-(quinoxalin-6-yl)-5′,6′-dihydrospiro[cyclohexane-1,7′-pyrrolo[1,2-a]imidazole]-4-yl methanesulfonate

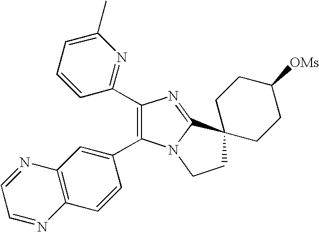

A 25-mL flask was charged with the compound from Example 12 (0.15 g, 0.36 mmol), methylene chloride (5 mL). Then methanesulfonic acid chloride (0.3 mL) and triethyl amine (1.0 mL) were added slowly. The resulting mixture was stirred at room temperature for 5 minutes. The mixture was diluted with methylene chloride and washed with brine. The crude product was purified by flash chromatography (silica gel, MeOH/methylene chloride 0:100 to 5:95) to give 0.14 g (80%) pure product.

### Step 13b: (1r,40-2′-(6-methylpyridin-2-yl)-3′-(quinoxalin-6-yl)-5′,6′-dihydrospiro[cyclohexane-1,7′-pyrrolo[1,2-a]imidazol]-4-amine

A 25-mL flask was charged with the mesylate of Step 11a (0.14 g, 0.29 mmol), sodium azide (0.20 g, 3.1 mmol) and DMF (5 mL). The mixture was heated at 65° C. for 12 hrs. The mixture was diluted with EtOAc and washed with brine. The crude product was purified by flash chromatography (silica gel, MeOH/methylene chloride 0:100 to 5:95) to give 94 mg (75%) of the intermediate azido compound (not shown). A 25-mL flask was charged with the azide similarly prepared to provide 0.10 g (0.23 mmol), palladium on charcoal (10%, 0.20 g) and methanol (5 mL). The mixture was stirred under one atmosphere of hydrogen for 12 hrs. The catalyst was filtered and the solvent was removed in vacuo to give a crude product which was purified by flash chromatography (silica gel, methanol/methylene chloride 0:100 to 5:95) to give 75 mg (71%) the title product.

MS (ES+) m/z 411.21 [MH+].

### Example 14

### (R)-2′-(6-methylpyridin-2-yl)-3′-(quinoxalin-6-yl)-5′,6′-dihydrospiro[cyclohex[3]ene-1,7′-pyrrolo[1,2-a]imidazole]

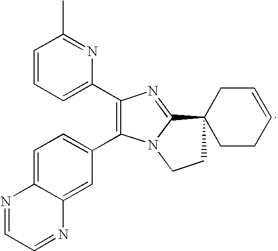

A mixture of the compound from Example 12 (41 mg, 0.1 mmol), toluene-4-sulfonic anhydride (42 mg, 0.13 mmol) and triethylamine (0.056 mL, 0.40 mmol) in methylene chloride (5 mL) was stirred at room temperature for 3 hrs. The solvent was concentrated and the residue dissolved in dimethyl sulfoxide (2 mL, 0.03 mol). Sodium hydride, 60% in mineral oil (excess ˜2.5 eq.) was added to the solution. The reaction was heated to 100° C. for 3 hrs. The mixture was cooled, diluted with water and extracted with ethyl acetate. The organic phases were dried and concentrated and the residue purified by HPLC to give the olefin, 16 mg.

¹H-NMR (300 MHz, CDCl₃) (J=Hz) δ 8.90 (s, 1H), 8.86 (s, 1H), 8.16 (s, 1H), 8.14 (d, 1H), 7.76 (m, 2H, J=9.0), 7.36 (d, 1H, J=7.8), 7.23 (d, 1H, J=7.8), 5.76 (dd, 2H), 4.16 (t, 2H, J=7.1), 2.70-1.80 (m, 11H). MS (ES+) m/z 393.95 [MH+].

### Example 15

### 3′-([1,2,4]triazolo[1,5-a]pyridin-6-yl)-2′-(5-fluoro-6-methylpyridin-2-yl)-1-(methylsulfonyl)-5′,6′-dihydrospiro[piperidine-4,7′-pyrrolo[1,2-a]imidazole] and 2′-([1,2,4]triazolo[1,5-a]pyridin-6-yl)-3′-(5-fluoro-6-methylpyridin-2-yl)-1-(methylsulfonyl)-5′,6′-dihydrospiro[piperidine-4,7′-pyrrolo[1,2-a]imidazole]

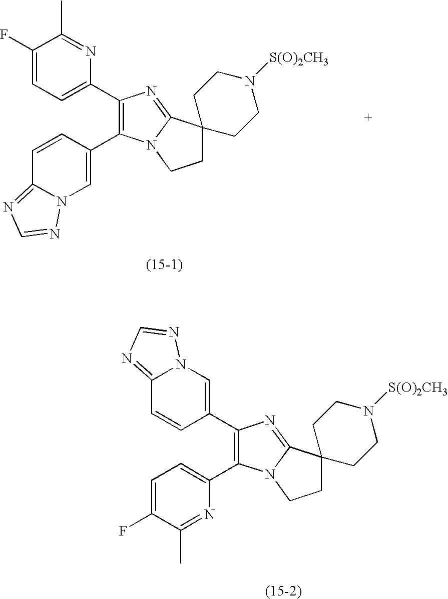

### Step 15a: tert-butyl 4-(5-([1,2,4]triazolo[1,5-a]pyridin-6-yl)-4-(5-fluoro-6-methylpyridin-2-yl)-1H-imidazol-2-yl)-4-(2-methoxyethyl)piperidine-1-carboxylate

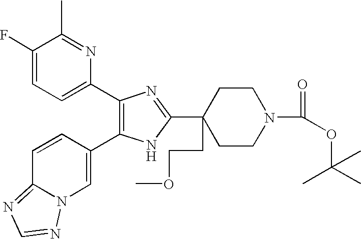

To a 500 ml flask was added 4-formyl-4-(2-methoxy-ethyl)-piperidine-1-carboxylic acid ten-butyl ester (2.5 g, 0.0092 mol), 1-(5-fluoro-6-methyl-pyridin-2-yl)-2-[1,2,4]triazolo[1,5-a]pyridin-6-yl-ethane-1,2-dione (3.1 g, 0.011 mol), ammonium acetate (5.4 g, 0.070 mol) and 2-methoxy-2-methylpropane (120 mL, 1.0 mol). To the resulting suspension was added Acetic acid (6 mL, 0.1 mol). The resulting mixture was heated to reflux overnight. The solvent was concentrated, diluted with sat. Na₂CO₃ aq. and extracted with CH₂Cl₂. The organic phases were dried and concentrated. The residue was chromatographed to give the title compound (0.70 g).

¹H-NMR (300 MHz, CDCl₃) (J=Hz) δ 9.04 (s, 1H), 8.42 (s, 1H), 7.80 (m, 2H), 7.316 (m, 2H), 3.70 (m, 2H), 3.55 (m, 2H) 3.40 (s; 3H), 2.95 (m; 2H), 2.60 (d, 3H), 2.5 (m, 2H), 2.2 (m, 2H), 1.94 (m, 2H), 1.48 (s; 9H). MS (ES+) m/z 536.4 [MH+].

### Step 15b: 6-(4-(5-fluoro-6-methylpyridin-2-yl)-2-(4-(2-methoxyethyl)piperidin-4-yl)-1H-imidazol-5-yl)[1,2,4]triazolo[1,5-a]pyridine

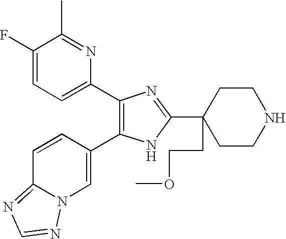

The product of Step 15a (0.64 g, 0.0012 mol) was dissolved in methylene chloride (20 mL, 0.3 mol) at RT. A solution of boron tribromide in hexane (1.0 M, 2.4 mL) was then added. The reaction was stirred for 3 hrs. Aqueous NaHCO₃ was added until pH=8-9. The mixture was washed with CH₂Cl₂ to remove the by-product. The remaining light yellow solid precipitate from the mixture was collected and dried to give the title compound (0.26 g, 52%).

MS (ES+) m/z 422.1 [MH+].

### Step 15c

The product of Step 15b (60.0 mg, 0.000142 mol) was dissolved in methylene chloride (5 mL, 0.08 mol) at RT. Methanesulphonic anhydride (50.0 mg, 0.000287 mol) was added to the solution. The reaction was stirred for 3 hrs and another 20 mg methanesulphonic anhydride was added and the solution was left over night. Aqueous NaHCO₃ was added to the solution until pH=8-9 and the mixture extracted with methylene chloride. The organic layer was dried and concentrated to give 48 mg crude product. The residue was subjected to HPLC (5-50% ACN) to give two regioisomers in the ratio of 15-c1:15-c2=2:1.

15-c1:

¹H-NMR (300 MHz, CDCl₃) (J=Hz) δ 8.97 (s, 1H), 8.41 (s, 1H), 7.87 (d, 1H), 7.60 (m, 2H), 7.29 (t, 1H), 4.15 (m, 2H), 3.65 (m, 2H), 3.28 (m, 2H), 2.81 (s, 3H), 2.65 (m, 3H), 2.55 (m, 1H), 2.32 (d, 3H), 2.30 (m; 2H), 1.90 (m, 2H). MS (ES+) m/z 482.27 [MH+].

15-c2:

¹H-NMR (300 MHz, CDCl₃) (J=Hz) δ 8.90 (s, 1H), 8.40 (s, 1H), 7.89 (d, 1H), 7.65 (d, 1H), 7.22 (m, 1H), 7.02 (m, 1H), 4.42 (t, 2H), 3.65 (m, 2H), 3.4 (m, 2H), 2.85 (s, 3H), 2.63 (m, 2H), 2.53 (d, 3H), 2.22 (m, 2H), 1.92 (m, 2H). MS (ES+) m/z 482.27 [MH+].

### Example 16

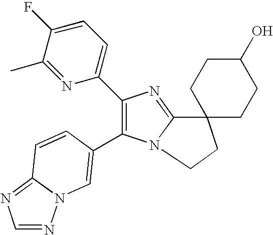

The title compound was prepared as described in Example 9. ¹H-NMR (300 MHz, MeOD), (J=Hz), δ 9.09 (s, 1H), 8.40 (s; 1H), 7.80 (m; 3H), 7.40 (t, 1H, J=9.0), 4.05 (t, 2H, J=6.9), 3.82 (m, 1H), 2.50 (t, 2H, J=6.9), 2.22 (d, 3H, J=2.7), 2.15 (m, 2H), 1.98 (m, 2H), 1.77 (m, 2H), 1.56 (m, 2H). MS (ES+) 419.10 [MH+].

### Example 17

### Methyl 2-(3′-([1,2,4]triazolo[1,5-a]pyridin-6-yl)-2′-(5-fluoro-6-methylpyridin-2-yl)-5′,6′-dihydrospiro[cyclohexane-1,7′-pyrrolo[1,2-a]imidazole]-4-ylidene)acetate

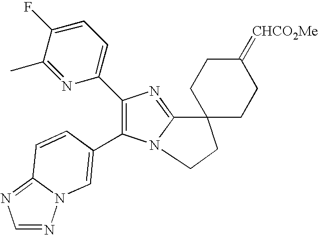

### Step 17a: 3′([1,2,4]triazolo[1,5-a]pyridin-6-yl)-2′-(5-fluoro-6-methylpyridin-2-yl)-5′,6′-dihydrospiro[cyclohexane-1,7′-pyrrolo[1,2-a]imidazol]-4-one

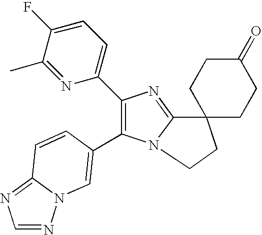

The compound from Example 16 was oxidized to the corresponding ketone following the procedure in Example 6.

### Step 17b

A mixture of the starting ketone (compound from step 17a) (51.0 mg, 0.122 mmol), (carbomethoxymethylene)-triphenylphosphorane (45 mg, 0.135 mmol), tetrahydrofuran (0.5 ml) and toluene (1.5 mL) was heated in a sealed tube at 150° C. for 4 hours. The mixture was cooled, concentrated to dryness and the residue dissolved in acetone/water and stirred for 48 hours. The mixture was concentrated and the residue purified by preparative HPLC to give the title product.

¹H-NMR (300 MHz, MeOD), (J=Hz), 9.17 (s, 1H), 8.47 (s; 1H), 7.86 (d, 1H, J=9.2), 7.71 (d, 1H, J=9.2), 7.38 (t, 1H, J=8.8), 7.23 (dd, 1H, J=8.6, J=3.5), 5.74 (s, 1H), 4.27 (t, 2H, J=7.1), 3.84 (m, 1H), 3.60 (s, 3H), 2.83 (t, 2H, J=7.1), 2.41 (m, 5H), 2.23 (m, 1H), 2.11-1.84 (m; 4H). MS (ES+) 472.99 [MH+].

### Example 18

### (1) Methyl 2-(3′-([1,2,4]triazolo[1,5-a]pyridin-6-yl)-4-amino-2′-(5-fluoro-6-methylpyridin-2-yl)-5′,6′-dihydrospiro[cyclohexane-1,7′-pyrrolo[1,2-a]imidazole]-4-yl)acetate and (2) 2-(3′-([1,2,4]triazolo[1,5-a]pyridin-6-yl)-4-amino-2′-(5-fluoro-6-methylpyridin-2-yl)-5′,6′-dihydrospiro[cyclohexane-1,7′-pyrrolo[1,2-a]imidazole]-4-yl)acetamide

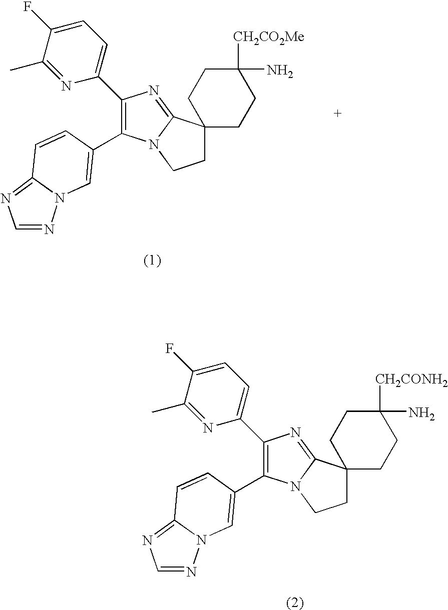

The unsaturated ester of Example 17 was dissolved in 7.0 M ammonia in methanol and the mixture stirred overnight at 40° C. The mixture was transferred to a sealed tube and purged with ammonia gas. The mixture was then heated 50° C. overnight, concentrated, and the residue purified by preparative HPLC to give the title compounds, an amino-amide (2) and an amino-ester (1).

MS (ES+): 478.8 [MH+] for the amino-amide, and 489.83 [MH+] for the amino-ester.

### Examples 19-72

Additional examples of compounds of the invention, i.e. Examples 19-72 as shown in Table 1, were prepared by known methods and methods described in the above examples. Table 2 contains physical data for compounds 19-72.

TABLE 2

| | | MS |
|---|---|---|
| Ex | H-NMR | (MH+) |
| 19 | (400 MHz, CDCl₃), (J = Hz), | 430.21 |
| | δ 7.91 (t, 1H, J = 8.0), | |
| | 7.37 (d; 1H, J = 8.0), | |
| | 7.17 (d; 1H, J = 8.4), | |
| | 7.00 (d, 1H, J = 7.6), | |
| | 6.84 (d; 1H; J = 8.4), | |
| | 6.80 (s; 1H), 6.15 (s; 2H), | |
| | 5.98 (m; 1H), 5.74 (m; 1H), | |
| | 4.48 (d, 2H, J = 5.2), | |
| | 3.96 (s, 1H), 2.86 (d, 2H, J = 5.2), | |
| | 2.83 (s; 3H), | |
| | 1.96 (m, 4H), 1.69 (m, 4H), | |
| | 1.53 (s; 1H). | |
| 20 | (400 MHz, DMSO-d6), (J = Hz), | 428.22 |
| | δ 9.01 (d, 2H, J = 8.8), | |
| | 8.24 (d, 1H, J = 8.4), | |
| | 8.25 (s, 1H), 7.18 (s, 1H), | |
| | 7.16 (t, 1H, J = 8.8), | |
| | 7.39 (d, 1H, J = 7.6), 7.05 (d, | |
| | 1H, J = 8.0), 5.90 (m, | |
| | 1H), 5.65 (m, 1H), | |
| | 4.54 (d, 2H, J = 8.8), 3.78 (m, | |
| | 1H), 2.76 (s, 3H), | |
| | 2.68 (m, 2H), 2.45 (d, 2H, J = 6.8), | |
| | 1.92 (m, 2H), | |
| | 1.66 (m, 2H), 1.55 (m, 2H). | |
| 21 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 22 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 23 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 24 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 25 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 26 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 27 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 28 | (300 MHz, CDCl₃), δ | 430.35 |
| | 8.20 (d, 1H), 7.55 (t, 1H), | |
| | 7.01 (d, 1H), 6.75 (m, | |
| | 2H), 6.11 (m, 1H), | |
| | 5.90 (m, 1H), 5.85 (s, 2H0, | |
| | 4.75 (d, 2H), 3.82 (m, | |
| | 1H), 2.70 (d, 2H), 2.60 (s, | |
| | 3H), 2.30 (m, 2H), | |
| | 2.1 (m, 2H), 1.9 (m, 2H), | |
| | 2.75 (m, 1H), 1.55 (m, | |
| | 2H) | |
| 29 | (400 MHz, MeOD), (J = Hz), | 409.97 |
| | δ 8.99 (d-d, 2H, J = 4.2, | |
| | 1.6), 8.36 (s, 1H), | |
| | 8.26 (d, 1H, J = 8.8), | |
| | 7.97 (d-d, 1H, J = 8.8, 2.0), | |
| | 7.86 (t, 1H, J = 8.2), | |
| | 7.46 (d, 1H, J = 7.8), 7.34 (d, | |
| | 1H, J = 8.0), 4.30 (t, 2H, | |
| | J = 7.0), 2.74 (m, 2H), | |
| | 2.72 (s, 3H), 2.18 (m, | |
| | 4H), 1.91 (m, 2H), | |
| | 2.03 (m, 2H), 1.70 (m, 2H). | |
| 30 | (400 MHz, MeOD), (J = Hz), | 409.97 |
| | δ 8.96 (s, 2H), | |
| | 8.33 (s, 1H), 8.20 (d, 1H, J = 8.8), | |
| | 7.92 (dd, 1H, J = 8.8, | |
| | 2.0), 7.66 (t, 1H, J = 8.2), | |
| | 7.35 (d, 1H, J = 8.0), | |
| | 7.23 (d, 1H, J = 8.4), | |
| | 4.66 (t, 2H, J = 7.6), 2.83 (t, | |
| | 2H, J = 8.0), 2.64 (t, 2H, | |
| | J = 8.0), 2.62 (s, 3H), | |
| | 2.28-1.66 (m, 6H). | |
| 31 | | 425.1 |
| 32 | (300 MHz, MeOD), (J = Hz), | 382.93 |
| | 9.14 (s, 1H), 8.46 (s; | |
| | 1H), 7.76 (m; 3H), | |
| | 7.55 (d, 1H, J = 7.8), 7.15 (d, | |
| | 1H, J = 7.6), 5.79 (m, 2H), | |
| | 4.19 (t, 2H, J = 6.25), | |
| | 2.58 (m, 3H), 2.37 (s, 3H), | |
| | 2.35-1.80 (m; 5H) | |
| 33 | (400 MHz, CDCl₃), (J = Hz), | 424.94 |
| | δ 8.77 (s, 2H), | |
| | 8.09 (s, 1H), 7.96 (d, 1H, J = 8.0), | |
| | 7.84 (d, 1H, J = 8.0), | |
| | 7.57 (d, 1H, J = 8.0), | |
| | 7.50 (t, 1H, J = 8.2), 6.92 (d, | |
| | 1H, J = 8.0), 4.05 (t, 2H, | |
| | J = 6.6), 3.44 (m, 1H), | |
| | 3.10 (m, 1H), 2.28 (s, | |
| | 3H), 2.48-1.75 (m, 8H). | |
| 34 | (300 MHz, CDCl₃), (J = Hz), | 404.1 |
| | δ 9.18 (s, 1H), | |
| | 8.51 (s, 1H), 7.83 (dd, 2H, | |
| | J = 9.0), 7.60-7.40 (m, | |
| | 2H), 4.25 (t, 2H, J = 7.1), | |
| | 3.70 (m, 2H), 3.35 (m, | |
| | 2H), 2.70 (m, 2H), | |
| | 2.34 (d, 3H), 2.30 (m; 2H), | |
| | 2.14 (m, 2H). | |
| 35 | (300 MHz, CD₃OD) δ | 404.1 |
| | 9.06 (s, 1H), 8.48 (s, 1H), | |
| | 7.80 (m, 2H), 7.48 (t, | |
| | 1H), 7.29 (dd, 1H), | |
| | 4.40 (t, 2H), 3.69 (m, 2H), | |
| | 3.32 (m, 2H), 2.72 (t, | |
| | 2H), 2.56 (d, 3H), | |
| | 2.3-2.1 (m, 4H) | |
| 36 | (300 MHz, CD₃OD), δ | 418.46 |
| | 9.21 (m, 1H), 8.54 (m, | |
| | 1H), 7.90-7.81 (m, 2H), | |
| | 7.62-745 (m, 2H), | |
| | 4.28 (m, 2H), 3.85-3.55 (m, | |
| | 3H), 3.10-2.95 (d, 2H), | |
| | 2.40 (d, 3H), | |
| | 2.78-2.05 (m, 8H) | |
| 37 | (300 MHz, CD₃OD) δ | 418.38 |
| | 9.23 (m, 1H), 8.53 (t, | |
| | 1H), 7.90-7.81 (m, 2H), | |
| | 7.62-745 (m, 2H), | |
| | 4.27 (m, 2H), 3.85-3.55 (m, | |
| | 3H), 3.10-2.95 (d, 2H), | |
| | 2.40 (d, 3H), | |
| | 2.78-2.05 (m, 8H) | |
| 38 | | 442.91 |
| 39 | | 398.99 |
| 40 | | 478.96 |
| 41 | | 413.35 |
| 42 | | 425.99 |
| 43 | | 400.01 |
| 44 | | 479.07 |
| 45 | | 443.97 |
| 46 | | 480.86 |
| 47 | (300 MHz, MeOD), | 447.1 |
| | 9.16 (s, 1H), 8.47 (s, 1H), | |
| | 7.86 (d, 1H), 7.71 (d, 1H), | |
| | 7.39 (t, 1H), 7.26 (m, | |
| | 1H), 4.26 (t, 2H), 2.41 (d, | |
| | 3H), 4.15-4.05 (m, 1H), | |
| | 3.66 (m, 1H), 2.76 (m, | |
| | 2H), 2.2-1.9 (m, 4H) | |
| 48 | | 435.84 |
| 49 | (300 MHz, CDCl₃), (J = Hz), | 400.9 |
| | d 8.93 (s, 1H), | |
| | 8.34 (s; 1H), 7.70 (d, 1H, | |
| | J = 9.0), 7.57 (d, 1H, | |
| | J = 9.0), 7.28 (m, 2H), | |
| | 5.70 (m, 2H), 4.05 (t, 2H, | |
| | J = 6.9), 2.46 (m, 6H), | |
| | 2.22 (d, 3H, J = 2.7), | |
| | 2.14 (m, 2H). | |
| 50 | (300 MHz, CDCl₃), | 504.1 |
| | 8.96 (s,, 1H), 8.34 (s, 1H), | |
| | 7.78-7.65 (m, 2H), | |
| | 7.25-7.10 (m, 2H), 4.05 (t, | |
| | 2H), 3.89 (m, 2H), | |
| | 3.86 (m, 2H), 2.53 (m, 2H), | |
| | 2.27 (d, 3H), 2.01 (m, | |
| | 2H), 1.73 (m, 2H), | |
| | 1.47 (s, 3H), 1.46 (s, 3H), | |
| | 1.45 (s, 3H) | |
| 51 | (300 MHz, CDCl₃), | 434.9 |
| | 9.19 (d, 1H), 8.47 (s, 1H), | |
| | 7.82-7.69 (m, 3H), | |
| | 7.47 (t, 1H), 4.3-3.8 (m, 4H), | |
| | 2.8-2.6 (m, 2H), 2.27 (dd, | |
| | 3H), 2.05-1.6 (m, 6H) | |
| 52 | | 409.95 |
| 53 | | 435.02 |
| 54 | (300 MHz, CDCl₃) δ | 476.1 |
| | 8.90 (s, 1H), 8.30 (s, 1H), | |
| | 7.77 (dd, 1H), 7.70 (d, 1H), | |
| | 7.61 (t, 1H), 7.25 (t, 1H), | |
| | 4.01 (t, 2H), 3.79 (s, 3H), | |
| | 3.66 (m, 2H), 3.4-3.2 (m, | |
| | br, 2H), 2.61-2.41 (m, | |
| | 4H), 2.23 (d, 3H), | |
| | 2.15-1.95 (2H0, 1.90-1.50 (m, | |
| | br, 2H) | |
| 55 | (300 MHz, CDCl₃) δ | 461.1 |
| | 8.90 (s, 1H), 8.31 (s, 1H), | |
| | 7.71-7.58 (m, 2H), | |
| | 7.26-7.14 (m, 2H), 3.96 (t, | |
| | 2H), 3.05-2.98 (m, br, | |
| | 4H), 2.50-2.43 (m, 4H), | |
| | 2.24 (d, 3H), 2.15 (m, | |
| | 2H), 1.75 (m, 2H) | |
| 56 | | 513.17 |
| 57 | | 416.96 |
| 58 | (300 MHz, MeOD), (J = Hz), | 427.98 |
| | 9.06 (s, 1H), 8.38 (s; | |
| | 1H), 7.69-7.50 (m, 4H), | |
| | 7.04 (d, 1H, J = 7.5), | |
| | 4.10 (t, 2H, J = 6.9), 2.59 (t, | |
| | 2H, J = 6.9), 2.33 (m, 1H), | |
| | 2.26 (s, 3H), | |
| | 2.07-1.58 (m; 8H) | |
| 59 | (300 MHz, CD₃OD) δ | 435.48 |
| | 8.91 (s, 1H), 8.37 (s, 1H), | |
| | 7.72 (d, 1H), 7.62 (d, | |
| | 1H), 7.38 (t, 1H), | |
| | 7.19 (dd, 1H), 4.13 (m, 3H), | |
| | 3.40 (m, 1H), 2.46 (m, | |
| | 5H), 2.19-1.52 (m, 6H) | |
| 60 | | 433.13 |
| 61 | (300 MHz, CD₃OD) δ | 402.9 |
| | 9.07 (s, 1H), 8.35 (s, 1H), | |
| | 7.78 (d, 1H), 7.70 (d, | |
| | 1H), 7.37 (m, 2H), | |
| | 2.7 (m, 1H), 2.55 (m, 1H), | |
| | 3.38 (d, 3H), | |
| | 2.05-1.25 (m, 12H) | |
| 62 | | 474.93 |
| 63 | ¹H-NMR (400 MHz, | 430.21 |
| | DMSO-d6), (J = Hz), δ | |
| | 7.79 (t, 1H, J = 8.4), | |
| | 7.28 (d; 1H, J = 8.0), 7.08 (d; | |
| | 1H, J = 8.4), 7.00 (d, 1H, | |
| | J = 7.6), 6.84 (d; 1H; J = 8.4), | |
| | 6.80 (s; 1H), 6.13 (s; | |
| | 2H), 6.03 (m; 1H), | |
| | 5.75 (m; 1H), 4.43 (d, 2H, J = 5.2), | |
| | 3.89 (m, 1H), | |
| | 3.51 (s, 1H), 2.81 (s; 3H), | |
| | 2.61 (d, 2H, J = 4.0), 2.22 (t, | |
| | 2H, J = 13.8), 2.08 (d, | |
| | 2H, J = 8.8), 1.98 (d; 2H, | |
| | J = 9.2), 1.58 (t, 2H, J = 11.6). | |
| 64 | ¹H-NMR (400 MHz, | 430.21 |
| | CDCl₃), (J = Hz), δ | |
| | 7.67 (t, 1H, J = 7.8), 7.28 (d; | |
| | 1H, J = 7.2), 7.09 (d; 1H, | |
| | J = 7.6), 6.89 (d, 1H, J = 8.4), | |
| | 6.76 (d; 1H; J = 8.4), | |
| | 6.74 (s; 1H), | |
| | 6.20 (m; 1H), 6.12 (m; 1H), | |
| | 5.97 (s; 2H), 4.84 (d, 2H, | |
| | J = 5.6), 3.90 (s, 1H), | |
| | 2.80 (d, 2H, J = 5.2), | |
| | 2.70 (s; 3H), 2.40 (t, 2H, J = 11.8), | |
| | 2.12 (d, 2H), 12.0), | |
| | 2.02 (d; 2H, J = 11.2), | |
| | 1.82 (s, 1H), 1.61 (t, 2H, | |
| | J = 8.8) | |
| 65 | ¹H-NMR (400 MHz, | 412.21 |
| | DMSO-d6), (J = Hz), δ | |
| | 8.99 (d-d, 2H, J = 6.4, | |
| | 2.4), 8.33 (d, 1H, J = 2.4), | |
| | 8.28 (d, 1H, J = 8.8), | |
| | 7.93 (d-d, 1H, J = 8.8, 2.0), | |
| | 7.82 (t, 1H, J = 8.2), | |
| | 7.39 (d, 1H, J = 8.0), 7.33 (d, | |
| | 1H, J = 8.4), 4.13 (t, 2H, | |
| | J = 7.6), 3.90 (m, 1H), | |
| | 2.73 (s, 3H), 2.61 (t, 2H, | |
| | J = 7.2), 2.27 (m, 2H), | |
| | 2.03 (m, 2H), 1.83 (m, | |
| | 2H), 1.80 (m, 2H). | |
| 66 | ¹H-NMR (400 MHz, | 412.21 |
| | DMSO-d6), (J = Hz), δ | |
| | 8.99 (d-d, 2H, J = 6.8, | |
| | 1.6), 8.33 (d, 1H, J = 1.2), | |
| | 8.28 (d, 1H, J = 8.8), | |
| | 7.93 (d-d, 1H, J = 8.8, 2.0), | |
| | 7.73 (t, 1H, J = 8.2), | |
| | 7.36 (d, 1H, J = 7.6), 7.30 (d, | |
| | 1H, J = 8.0), 4.17 (t, 2H, | |
| | J = 7.6), 3.72 (m, 1H), | |
| | 2.67 (t, 2H, J = 7.2), | |
| | 2.65 (s, 3H), 2.07 (m, 2H), | |
| | 2.03 (m, 2H), 1.97 (m, | |
| | 2H), 1.47 (m, 2H). | |
| 67 | ¹H-NMR (400 MHz, | 459.22 |
| | DMSO-d6), (J = Hz), δ | |
| | 8.44 (d, 1H, J = 2.0), | |
| | 8.25 (s, 1H), 7.92 (d, 1H, J = 8.4), | |
| | 7.84 (d, 1H, J = 8.8), | |
| | 7.37 (t, 1H, J = 8.8), | |
| | 7.27 (d, 1H, J = 8.8), 4.18 (t, | |
| | 2H, J = 6.2), 3.55 (s, 3H), | |
| | 3.35 (m, 1H), 2.71 (t, 2H, | |
| | J = 6.8), 2.47 (s, 3H), | |
| | 2.20 (m, 4H), 2.06 (m, | |
| | 2H), 1.72 (m, 2H). | |
| 68 | ¹H-NMR (300 MHz, | 401.0 |
| | MeOD), (J = Hz), δ | |
| | 9.03 (s, 1H), 8.35 (s; 1H), | |
| | 7.74 (m; 2H), 7.60 (m, 1H), | |
| | 7.24 (m; 2H), 4.03 (t, 2H, | |
| | J = 7.2), 3.77 (m, 1H), | |
| | 2.50 (m, 5H), 2.08 (m, | |
| | 2H), 1.80 (m, 2H), | |
| | 1.67 (m, 2H), 1.55 (m, 2H). | |
| 69 | ¹H-NMR (300 MHz, | 401.0 |
| | CDCl₃), (J = Hz), δ | |
| | 9.25 (s, 1H), 8.58 (s; 1H), | |
| | 7.94 (m; 1H), 7.75 (m, 2H), | |
| | 7.33 (m; 2H), 4.68 (t, 2H, | |
| | J = 7.2), 4.05 (m, 1H), | |
| | 2.70 (m, 5H), | |
| | 2.40-1.70 (m, 8H) | |
| 70 | ¹H-NMR (400 MHz, | 412.21 |
| | DMSO-d6), (J = Hz), δ | |
| | 8.93 (d, 2H, J = 6.4), | |
| | 8.34 (s, 1H), 8.24 (d, 1H, J = 8.8), | |
| | 7.92 (d, 1H, J = 8.8), | |
| | 7.82 (t, 1H, J = 8.2), | |
| | 7.39 (d, 1H, J = 8.0), 7.15 (d, | |
| | 1H, J = 8.4), 4.57 (t, 2H, | |
| | J = 7.6), 3.89 (m, 1H), | |
| | 2.71 (t, 2H, J = 7.2), | |
| | 2.60 (s, 3H), 2.42 (m, 2H), | |
| | 1.82 (m, 2H), 1.76 (m, | |
| | 2H), 1.50 (m, 2H). | |
| 71 | ¹H-NMR (400 MHz, | 442.22 |
| | CDCl₃), (J = Hz), δ | |
| | 8.47 (d, 1H, J = 10.8), 7.97 (d, | |
| | 1H, J = 7.2), 7.81 (d, 1H, | |
| | J = 8.0), 7.53 (t, 1H, J = 8.0), | |
| | 7.28 (s, 1H), 7.24 (d, | |
| | 1H, J = 7.2), 7.12 (t, 1H, | |
| | J = 8.0), 4.60 (t, 2H, J = 6.8), | |
| | 4.00 (m, 1H), | |
| | 3.61 (s, 3H), 2.71 (t, 2H, J = 7.2), | |
| | 2.64 (s, 3H), 2.48 (t, | |
| | 2H, J = 10.2), 2.00 (m, | |
| | 2H), 1.87 (m, 2H), | |
| | 1.73 (m, 2H). | |
| 72 | ¹H-NMR (400 MHz, | 411.22 |
| | DMSO-d6), (J = Hz), δ | |
| | 8.93 (d, 2H, J = 6.4), | |
| | 8.34 (s, 1H), 8.24 (d, 1H, J = 8.8), | |
| | 7.92 (d, 1H, J = 8.8), | |
| | 7.82 (t, 1H, J = 8.2), | |
| | 7.39 (d, 1H, J = 8.0), 7.15 (d, | |
| | 1H, J = 8.4), 4.57 (t, 2H, | |
| | J = 7.6), 3.89 (m, 1H), | |
| | 2.71 (t, 2H, J = 7.2), | |
| | 2.60 (s, 3H), 2.42 (m, 2H), | |
| | 1.82 (m, 2H), 1.76 (m, | |
| | 2H), 1.50 (m, 2H). | |

The TGFβ inhibitory activity of compounds of formula (I) can be assessed by methods described in the following examples.

### Example 73

### Cell-Free Assay for Evaluating Inhibition of TGFβ

Autophosphorylation of TGFβ Type I Receptor. The serine-threonine kinase activity of TGFβ type I receptor was measured as the autophosphorylation activity of the cytoplasmic domain of the receptor containing an N-terminal polyhistidine, TEV cleavage site-tag, e.g., His-TGFβRI. The His-tagged receptor cytoplasmic kinase domains were purified from infected insect cell cultures using the Gibco-BRL FastBac HTb baculovirus expression system.

To a 96-well Nickel FlashPlate (NEN Life Science, Perkin Elmer) was added 20 μl of 1.25 μCi ³³P-ATP/25 μM ATP in assay buffer (50 mM Hepes, 60 mM NaCl, 1 mM MgCl₂, 2 mM DTT, 5 mM MnCl₂, 2% glycerol, and 0.015% Brij® 35). 10 μl of each test compound of formula (I) prepared in 5% DMSO solution were added to the FlashPlate. The assay was then initiated with the addition of 20 ul of assay buffer containing 12.5 ρmol of His-TGFβRI to each well. Plates were incubated for 30 minutes at room temperature and the reactions were then terminated by a single rinse with TBS. Radiation from each well of the plates was read on a TopCount (Packard). Total binding (no inhibition) was defined as counts measured in the presence of DMSO solution containing no test compound and non-specific binding was defined as counts measured in the presence of EDTA or no-kinase control.

Alternatively, the reaction performed using the above reagents and incubation conditions but in a microcentrifuge tube was analyzed by separation on a 4-20% SDS-PAGE gel and the incorporation of radiolabel into the 40 kDa His-TGFβRI SDS-PAGE band was quantitated on a Storm Phosphoimager (Molecular Dynamics).

Compounds of formula (I) typically exhibited IC₅₀ values of less than 10 μM; some exhibited IC₅₀ values of less than 1 μM; and some even exhibited IC₅₀ values of less than 50 nM.

### Example 74

### Cell-Free Assay for Evaluating Inhibition of Activin Type I Receptor Kinase Activity

Inhibition of the Activin type I receptor (Alk4) kinase autophosphorylation activity by test compounds of formula (I) can be determined in a similar manner to that described above in Example 94 except that a similarly His-tagged form of Alk4 (His-Alk 4) is used in place of the His-TGFβRI.

### Example 75

### TGFβ Type I Receptor Ligand Displacement FlashPlate Assay

50 nM of tritiated 4-(3-pyridin-2-yl-1H-pyrazol-4-yl)-quinoline (custom-ordered from PerkinElmer Life Science, Inc., Boston, Mass.) in assay buffer (50 mM Hepes, 60 mM NaCl₂, 1 mM MgCl₂, 5 mM MnCl₂, 2 mM 1,4-dithiothreitol (DTT), 2% Brij® 35; pH 7.5) was premixed with a test compound of formula (I) in 1% DMSO solution in a v-bottom plate. Control wells containing either DMSO without any test compound or control compound in DMSO were used. To initiate the assay, His-TGFβ Type I receptor in the same assay buffer (Hepes, NaCl₂, MgCl₂, MnCl₂, DTT, and 30% Brij® added fresh) was added to a nickel coated FlashPlate (PE, NEN catalog number: SMP107), while the control wells contained only buffer (i.e., no His-TGFβ Type I receptor). The premixed solution of tritiated 4-(3-pyridin-2-yl-1H-pyrazol-4-yl)-quinoline and test compound of formula (I) was then added to the wells. The wells were aspirated after an hour at room temperature and radioactivity in wells (emitted from the tritiated compound) was measured using TopCount (PerkinElmer Lifesciences, Inc., Boston Mass.).

Compounds of formula (I) typically exhibited IC; values of less than 10 μM; some exhibited Kᵢ values of less than 1 μM; and some even exhibited Kᵢ values of less than 50 nM.

### Example 76

### Assay for Evaluating Cellular Inhibition of TGFβ Signaling and Cytotoxicity

Biological activity of the compounds of formula (I) was determined by measuring their ability to inhibit TGFβ-induced PAI-Luciferase reporter activity in HepG2 cells.

HepG2 cells were stably transfected with the PAI-luciferase reporter grown in DMEM medium containing 10% FBS, penicillin (100 U/ml), streptomycin (100 μg/ml), L-glutamine (2 mM), sodium pyruvate (1 mM), and non-essential amino acids (1×). The transfected cells were then plated at a concentration of 2.5×104 cells/well in 96 well plates and starved for 3-6 hours in media with 0.5% FBS at 37° C. in a 5% CO₂ incubator. The cells were then stimulated with 2.5 ng/ml TGFβ ligand in the starvation media containing 1% DMSO either in the presence or absence of a test compound of formula (I) and incubated as described above for 24 hours. The media was washed out the following day and the luciferase reporter activity was detected using the LucLite Luciferase Reporter Gene Assay kit (Packard, cat. no. 6016911) as recommended. The plates were read on a Wallac Microbeta plate reader, the reading of which was used to determine the IC₅₀ values of compounds of formula (I) for inhibiting TGFβ-induced PAI-Luciferase reporter activity in HepG2 cells. Compounds of formula (I) typically exhibited IC₅₀ values of less 10 uM.

Cytotoxicity was determined using the same cell culture conditions as described above. Specifically, cell viability was determined after overnight incubation with the CytoLite cell viability kit (Packard, cat. no. 6016901). Compounds of formula (I) typically exhibited LD₂₅ values greater than 10 μM.

### Example 77

### Assay for Evaluating Inhibition of TGFβ Type I Receptor Kinase Activity in Cells

The cellular inhibition of activin signaling activity by the test compounds of formula (I) is determined in a similar manner as described above in Example 97 except that 100 ng/ml of activin is added to serum starved cells in place of the 2.5 ng/ml TGFβ.

### Example 78

### Assay for TGFβ-Induced Collagen Expression

### Preparation of Immortalized Collagen Promotor-Green Fluorescent Protein Cells

Fibroblasts are derived from the skin of adult transgenic mice expressing Green Fluorescent Protein (GFP) under the control of the collagen 1A1 promoter (see Krempen, K. et al., Gene Exp. 8: 151-163 (1999)). Cells are immortalized with a temperature sensitive large T antigen that is in an active stage at 33° C. Cells are expanded at 33° C. and then transferred to 37° C. at which temperature the large T antigen becomes inactive (see Xu, S. et al., Exp. Cell Res., 220: 407-414 (1995)). Over the course of about 4 days and one split, the cells cease proliferating. Cells are then frozen in aliquots sufficient for a single 96 well plate.

Assay of TGFβ-induced Collagen-GFP Expression

Cells are thawed, plated in complete DMEM (contains non-essential amino acids, 1 mM sodium pyruvate and 2 mM L-glutamine) with 10% fetal calf serum, and then incubated for overnight at 37° C., 5% CO₂. The cells are trypsinized in the following day and transferred into 96 well format with 30,000 cells per well in 50 pa complete DMEM containing 2% fetal calf serum, but without phenol red. The cells are incubated at 37° C. for 3 to 4 hours to allow them to adhere to the plate. Solutions containing a test compound of formula (I) are then added to wells with no TGFβ (in triplicates), as well as wells with 1 ng/ml TGFβ (in triplicates). DMSO is also added to all of the wells at a final concentration of 0.1%. GFP fluorescence emission at 530 nm following excitation at 485 nm is measured at 48 hours after the addition of solutions containing a test compound on a CytoFluor microplate reader (PerSeptive Biosystems). The data are then expressed as the ratio of TGFβ-induced to non-induced for each test sample.

### Example 79

### Assay for Evaluating Inhibition and/or Prevention of Restinosis

Stenotic fibrotic Response Balloon Catheter Injury of the Rat Carotid Artery.

The ability of compounds of formula (I) to prevent the stenotic fibrotic response is tested by administration of the test compounds to rats that have undergone balloon catheter injury of the carotid artery. The test compounds are administered intravenously, subcutaneously or orally.

Sprague Dawley rats (400 g, 3 to 4 months old) are anesthetized by inter paratenal i.p. injection with 2.2 mg/kg xylazine (AnaSed, Lloyd laboratories) and 50 mg/kg ketamine (Ketalar, Parke-Davis). The left carotid artery and the aorta are denuded with a 2F balloon catheter according to the procedure described in Clowes et al., Lab Invest., 49: 327-333 (1983). Test compounds of formula (I) are each administered to the treatment group (n=5-10 rats) (i.v., p.o., or s.c.; qod, once per day, bid, tid or by continuous s.c. infusion via an Alzet minipump) starting the day of surgery and subsequently for 14 more days. The control group (n=5 rats) received the same volume of vehicle administered using the same regimen as the test compound-treated rats. The animals were sacrificed under anesthesia 14 days post-balloon injury. Perfusion fixation was carried out under physiological pressure with phosphate buffered (0.1 mol/L, pH 7.4) 4% paraformadehyde. The injured carotid artery was excised, post-fixed and embedded for histological and morphometic analysis. Sections (5 μm) were cut from the proximal, middle and distal segments of the denuded vessel and analyzed using image analysis software. The circumference of the lumen and the lengths of the internal elastic lamina (IEL) and the external elastic lamina (EEL) were determined by tracing along the luminal surface the perimeter of the neointima (EEL) and the perimeter of the tunica media (EEL) respectively. The lumen (area within the lumen), medial (area between the EEL and EEL) and intimal (area between the lumen and the IEL) areas were also determined using morphometric analysis. Statistical analysis used ANOVA to determine statistically significant differences between the means of treatment groups (p≦0.05). Multiple comparisons between groups were then performed using the Scheffe test. The Student t test was used to compare the means between 2 groups, and differences were considered significant if P≦0.05. All data are shown as mean±SEM.

Statistically significant decreases were seen in intimal area, intimal/medial ratio in injured arteries of test compound-treated rats compared to those of the vehicle-treated rats. Conversely, the lumen area, IEL and EEL lengths showed a statistically significant increase in injured arteries of test compound-treated rats compared to those of the vehicle-treated rats. These results show that inhibition of the TGFβRI kinase prevents the stenotic response to balloon-catheter arterial injury by inhibiting the fibrotic expansion of the neointima and vessel remodeling.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound of formula I 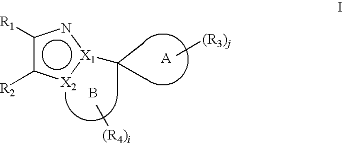
wherein:
R₁ is aryl or heteroaryl, and is optionally substituted with 1 to 3 Rₐ;
R₂ is aryl or heteroaryl, and is optionally substituted with 1 to 3 R_{b};
Ring A is a 5- to 8-membered cycloaliphatic, or a 5- to 8-membered heterocycloaliphatic containing one to three heteroatoms;
Ring B is a 5- to 8-membered heterocycloaliphatic containing one to three heteroatoms;
each of Rₐ and R_{b} is independently an aliphatic, alkoxy, acyl, halo, hydroxy, amino, amido, nitro, cyano, guanadino, amidino, carboxy, sulfo, sulfinyl, sulfonyl, sunfanyl, alkoxycarbonyl, alkylcarbonyloxy, urea, thiourea, sulfamoyl, sulfamide, carbamoyl, cycloalkyl, cycloalkyloxy, heterocycloalkyl, heterocycloalkyloxy, aryl, aryloxy, aroyl, heteroaryl, heteroaryloxy, heteroaroyl, oxo, thioxo, ═N—OR_{f}, —N₃, or ═N—N(R_{f})₂; or
any two of Rₐ or any two of R_{b} on adjacent atoms, together with the atoms to which they are attached, may form a 3- to 8-membered cycloaliphatic or a 3- to 8-membered heterocycloaliphatic;
each of R₃ and R₄, if present, is independently an aliphatic, acyl, alkoxy, cycloalkyloxy, heterocycloalkyloxy, aryloxy, heteroaryloxy, aralkyloxy, heteroarylalkoxy, amino, amido, nitro, carboxy, cyano, halo, hydroxyl, sulfanyl, sulfinyl, sulfonyl, urea, thiourea, sulfamoyl, sulfamide, oxo, thioxo, —N₃, ═N—OR_{f}, or ═N—N(R_{f})₂; or
any two of R₃ or any two of R₄ on the same atom, together with the atom to which they are attached, may form a 3- to 8-membered cycloaliphatic or heterocycloaliphatic ring, or
any two of R₃ or any two of R₄ on adjacent atoms, together with the atoms to which they are attached, may form a 3- to 8-membered cycloaliphatic or a 3- to 8-membered heterocycloaliphatic ring;
each of the optional heteroatoms of Rings A and B is independently O, S, or N, and the S and N atoms in Rings A and B may be part of the groups —S(O)ₘ— and —N(R_{f})—;
each R_{f} is independently H, alkyl, heteroaryl, aryl, acyl, aroyl, heteroaroyl, amido, sulfamoyl, sulfamide, or carboxy;
X₁ is C and X₂ is N, or X₁ is N and X₂ is C;
each i is independently an integer from 0 to 3;
each j is independently an integer from 0 to 3; and
each m is independently an integer from 0 to 2.

2. A compound of claim 1, wherein X₁ is C and X₂ is N.

3. The compound of claim 2, wherein R₁ is heteroaryl optionally substituted with 1 to 3 Rₐ.

4. The compound of claim 3, wherein R₂ is heteroaryl optionally substituted with 1 to 3 R_{b}.

5. The compound of claim 1, wherein R₁ is aryl optionally substituted with 1 to 3 Rₐ; and R₂ is heteroaryl optionally substituted with 1 to 3 R_{b}.

6. The compound of claim 1, wherein R₁ is heteroaryl optionally substituted with 1 to 3 Rₐ; and R₂ is aryl optionally substituted with 1 to 3 R_{b}.

7. The compound of claim 1, wherein R₁ is pyridinyl or pyrimidinyl, and is optionally substituted with 1 to 3 Rₐ.

8. The compound of claim 7, wherein R₁ is pyridinyl optionally substituted with 1 to 3 Rₐ.

9. The compound of claim 8, wherein R₁ is pyridin-2-yl substituted with at least one Rₐ.

10. The compound of claim 9, wherein R₁ is 6-methyl-pyridin-2-yl.

11. The compound of claim 1, wherein R₂ is a bicyclic heteroaryl optionally substituted with 1 to 3 R_{b}.

12. The compound of claim 11, wherein the bicyclic heteroaryl is

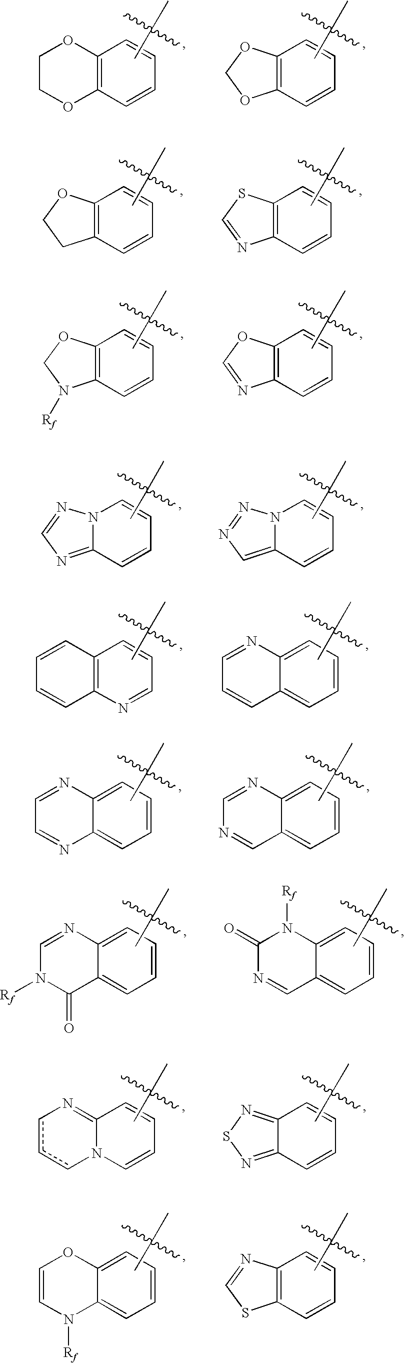

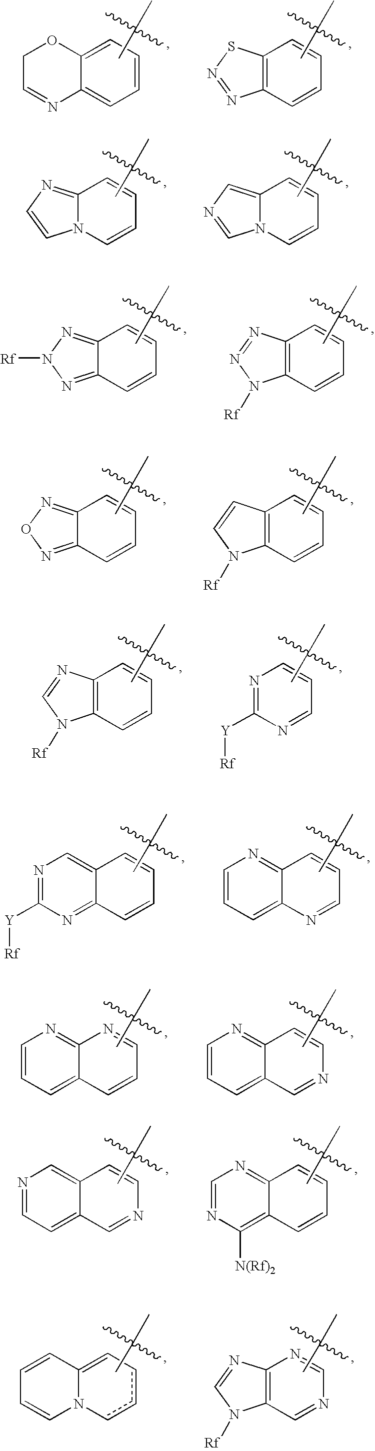

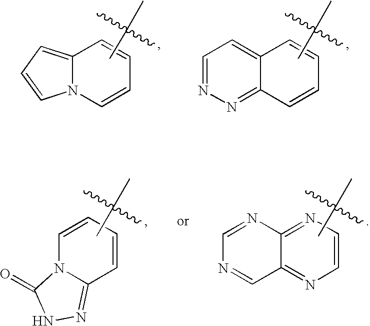

13. The compound of claim 11, wherein R₂ is benzodioxolyl, quinazolinyl, or imidazopyridinyl, each optionally substituted with 1 to 3 R_{b}.

14. The compound of claim 13, wherein R₁ is pyridinyl or pyrimidinyl, each optionally substituted with 1 to 3 Rₐ.

15. The compound of claim 1, wherein Ring B is a 5- to 7-membered heterocycloaliphatic.

16. The compound of claim 15, wherein Ring A is a 5- or 6-membered cycloalkyl optionally substituted with one of alkoxy, amino, oxo, —N₃, or ═N—OR_{f}.

17. The compound of claim 15, wherein Ring A is an optionally substituted 5- to 8-membered cycloaliphatic.

18. The compound of claim 17, wherein Ring A is an optionally substituted 5- or 6-membered cycloaliphatic.

19. The compound of claim 15, wherein Ring A is an optionally substituted 5- to 8-membered heterocycloaliphatic.

20. The compound of claim 19, wherein Ring A is an optionally substituted piperidine.

21. The compound of claim 20, wherein the piperidine nitrogen ring atom is substituted with R_{f}.

22. The compound of claim 20, wherein the piperidine is substituted with at least one oxo substituent.

23. The compound of claim 22, wherein the oxo substituent is attached to a carbon atom adjacent to the piperidine nitrogen ring atom.

24. The compound of claim 15, wherein Ring A is a 7-membered heterocycloaliphatic.

25. The compound of claim 24, wherein Ring A includes a nitrogen atom.

26. The compound of claim 25, wherein Ring A is substituted with at least one oxo substituent at a carbon atom adjacent to the ring nitrogen atom.

27. The compound of claim 15, wherein Ring B is a 5- to 7-membered heterocycloalkenyl.

28. The compound of claim 27, wherein Ring B is a 5-membered heterocycloalkenyl.

29. The compound of claim 27, wherein Ring B is a 6-membered heterocycloalkenyl.

30. The compound of claim 27, wherein Ring B is a 7-membered heterocycloalkenyl.

31. The compound of claim 30, wherein X₁ is C and X₂ is N.

32. The compound of claim 1, wherein X₁ is N and X₂ is C.

33. A compound of formula I 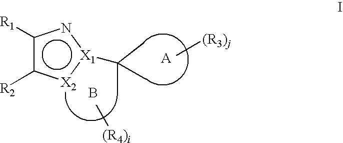
wherein
X₁ is C and X₂ is N,
R₁ is 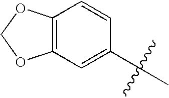
and R₂ is 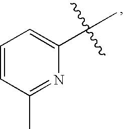
or
R₁ is 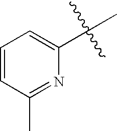
and R₂ is 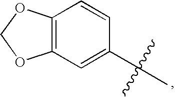
Ring B is 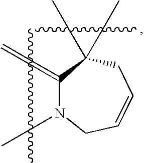
and
Ring A is

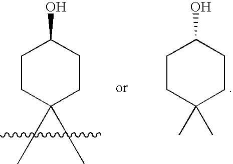

34. A compound of formula I 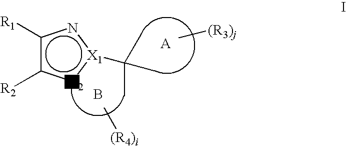
wherein
X₁ is C and X₂ is N,
R₁ is 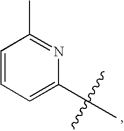
R₂ is 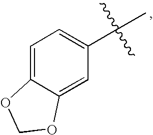
Ring B is 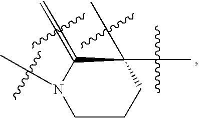
and
Ring A is

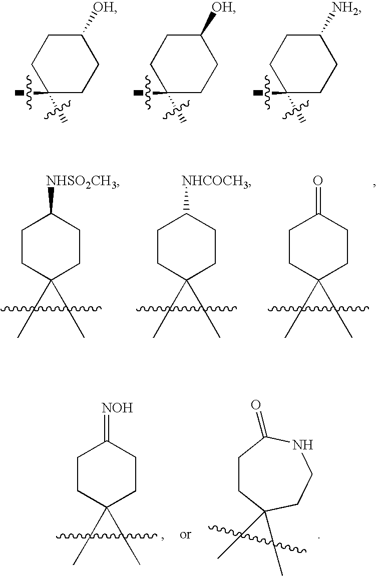

35. A compound of formula I 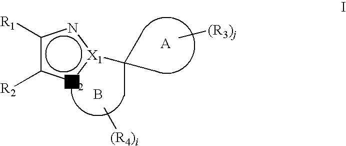
wherein
X₁ is C and X₂ is N,
R₁ is 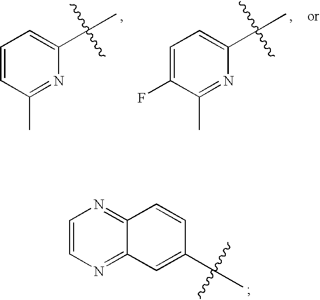
R₂ is 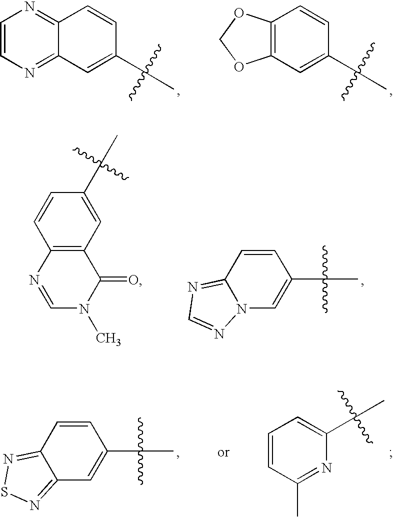
Ring B is 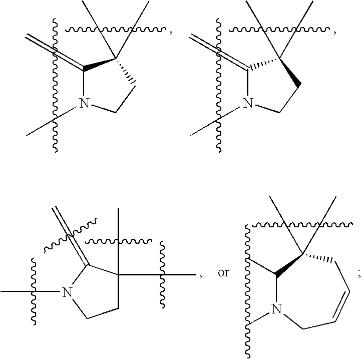
and
Ring A is

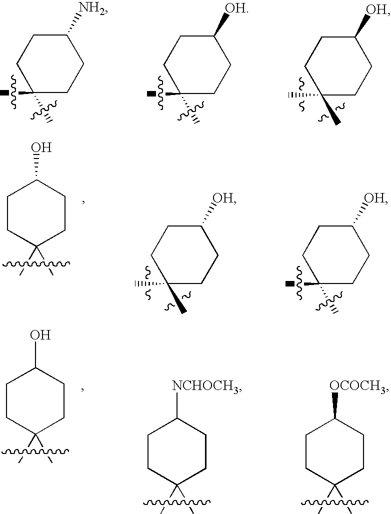

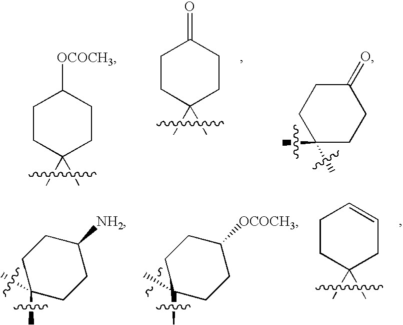

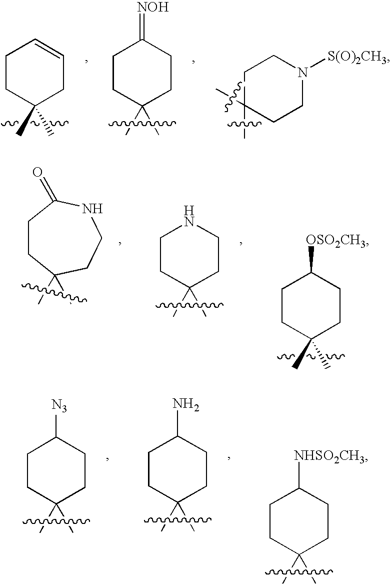

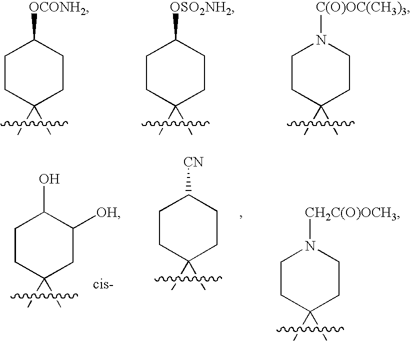

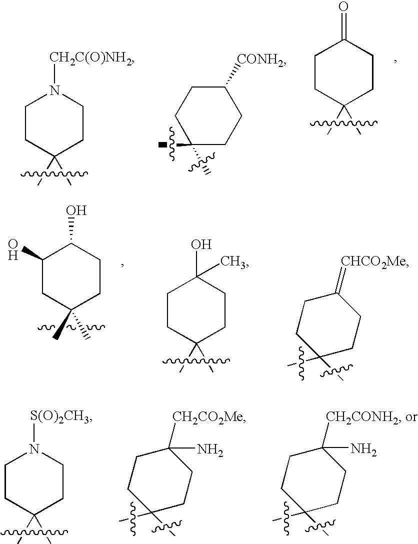

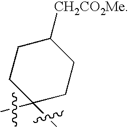

36. A compound of formula I 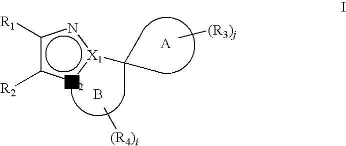
wherein
X₁ is C and X₂ is N,
R₁ is 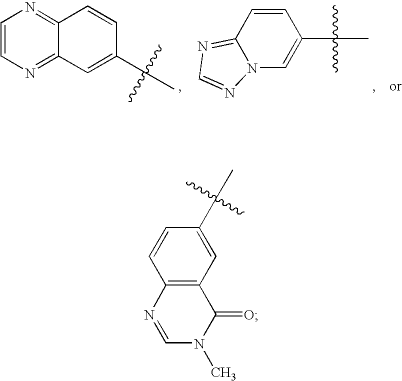
R₂ is 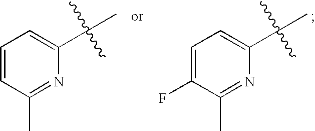
Ring B is 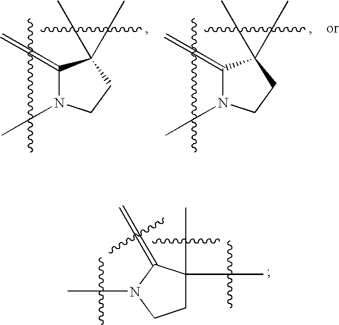
and
Ring A is

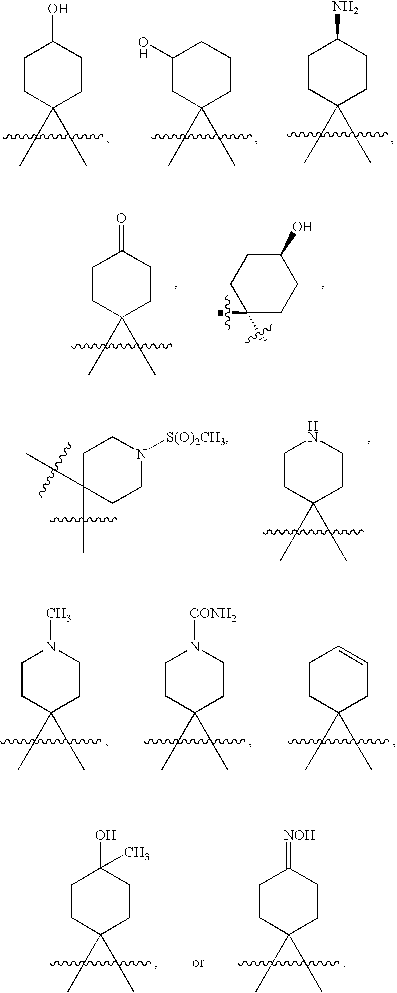

37. A pharmaceutical composition comprising a compound of claim 1 and a pharmaceutically acceptable carrier.

38. An implantable device comprising a compound of claim 1.

39. A method of inhibiting the TGFβ signaling pathway in a subject, comprising administering to said subject in need thereof an effective amount of a compound of claim 1.

40. A method of inhibiting the TGFβ type I receptor in a cell, comprising contacting said cell with an effective amount of a compound of claim 1.

41. A method of reducing the accumulation of excess extracellular matrix induced by TGFβ in a subject, comprising administering to said subject in need thereof an effective amount of a compound of claim 1.

42. A method of treating or preventing fibrotic condition in a subject, comprising administering to said subject in need thereof an effective amount of a compound of claim 1.

43. The method of claim 42, wherein the fibrotic condition is selected from the group consisting of mesothelioma, acute respiratory distress syndrome (ARDS), atherosclerosis, scleroderma, keloids, glomerulonephritis, diabetic nephropathy, lupus nephritis, hypertension-induced nephropathy, cholangitis, restenosis, ocular scarring, corneal scarring, hepatic fibrosis, liver cirrhosis, cirrhosis due to fatty liver disease (alcoholic and nonalcoholic steatosis), biliary fibrosis, pulmonary fibrosis, renal fibrosis, sarcoidosis, acute lung injury, drug-induced lung injury, spinal cord injury, central nervous system scarring, systemic lupus erythematosus, Wegener's granulomatosis, cardiac fibrosis, post-infarction cardiac fibrosis, post-surgical fibrosis, connective tissue disease, radiation-induced fibrosis, chemotherapy-induced fibrosis, transplant arteriopathy, fibrosclerosis, fibrotic cancers, fibroids, fibroma, fibroadenomas, and fibrosarcomas.

44. A method of inhibiting metastasis of tumor cells in a subject, comprising administering to said subject in need thereof an effective amount of a compound of claim 1.

45. A method of treating carcinomas mediated by an overexpression of TGFβ, comprising administering to a subject in need thereof an effective amount of a compound of claim 1.

46. The method of claim 45, wherein said carcinomas are selected from the group consisting of carcinomas of the lung, breast, liver, biliary tract, gastrointestinal tract, head, neck, pancreas, prostate, and cervix, multiple myeloma, melanoma, glioma, and glioblastomas.

47. A method of treating or preventing a restenosis, vascular disease, or hypertension by administering to a subject in need thereof a compound of claim 1.

48. The method of claim 47, wherein the restinosis is coronary restenosis, peripheral restenosis, or carotid restenosis.

49. The method of claim 47, wherein the vascular disease is intimal thickening, vascular remodeling, or an organ transplant-related vascular disease.

50. The method of claim 49, wherein the vascular disease is intimal thickening or vascular remodeling.

51. The method of claim 47, wherein the hypertension is systolic hypertension, pulmonary hypertension, or hypertension-induced vascular remodeling.
